SEQUENCE LISTING (1 sequence)

SEQ ID NO: 1 - protein, 165 aa, homo sapiens
  VARIANT (0)...(0): Erythropoietin (EPO)
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGD

(19) United States
(12) Patent Application Publication
(10) Pub. No.: US 2008/0207487 A1
(43) Pub. Date: Aug. 28, 2008

(54) MANUFACTURING PROCESS FOR THE PRODUCTION OF POLYPEPTIDES EXPRESSED IN INSECT CELL-LINES

(75) Inventors: Shawn DeFrees, North Wales, PA (US); Kyle Kinealy, Plymouth Meeting, PA (US); Sibylle Herzer, Flemington, NJ (US)

Correspondence Address: MORGAN, LEWIS & BOCKIUS LLP (SF), One Market, Spear Street Tower, Suite 2800, San Francisco, CA, 94105, US

(73) Assignee: Neose Technologies, Inc., Horsham, PA (US)

(21) Appl. No.: 11/934,700

(22) Filed: Nov. 2, 2007

Related U.S. Application Data
(60) Provisional application No. 60/864,117, filed on Nov. 2, 2006.
(60) Provisional application No. 60/868,057, filed on Nov. 30, 2006.
(60) Provisional application No. 60/887,517, filed on Jan. 31, 2007.
(60) Provisional application No. 60/951,159, filed on Jul. 20, 2007.
(60) Provisional application No. 60/955,001, filed on Aug. 9, 2007.
(60) Provisional application No. 60/956,468, filed on Aug. 17, 2007.
(60) Provisional application No. 60/978,298, filed on Oct. 8, 2007.

(51) Int. Cl. A61K 38/02; C07K 1/16; C07K 14/505; A61P 7/06; C12P 21/00; C12N 15/866

(52) U.S. Cl. 514/2; 530/338; 530/397; 435/456; 435/69.1

(57) ABSTRACT

The present invention provides a manufacturing method for polypeptides that are produced in insect cells using a baculoviral expression system. In one example, the insect cell culture is supplemented with a lipid mixture immediately prior to infection (e.g., one hour prior to infection). The polypeptides are isolated from the insect cell culture using a method that employs anion exchange or mixed-mode chromatography early in the purification process. This process step is useful to remove insect-cell derived endoglycanases and proteases and thus reduces the loss of desired polypeptide due to enzymatic degradation. In another example, mixed-mode chromatography is combined with dye-ligand affinity chromatography in a continuous-flow manner to allow for rapid processing of the insect-cell culture liquid and capture of the polypeptide. In yet another example, a polypeptide is isolated from an insect cell culture liquid using a process that combines hollow fiber filtration, mixed-mode chromatography and dye-ligand affinity in a single unit operation producing a polypeptide solution that is essentially free of endoglycanase and proteolytic activities. In a further example, the isolated polypeptides are glycopeptides having an insect specific glycosylation pattern, which are optionally conjugated to a modifying group, such as a polymer (e.g., PEG) using a glycosyltransferase and a modified nucleotide sugar.

### CROSS-REFERENCES TO RELATED APPLICATIONS

The present application claims priority to U.S. Provisional Patent Application No. 60/864,117, filed on Nov. 2, 2006; U.S. Provisional Patent Application No. 60/868,057, filed on Nov. 30, 2006; U.S. Provisional Patent Application No. 60/887,517, filed on Jan. 31, 2007; U.S. Provisional Patent Application No. 60/951,159, filed on Jul. 20, 2007; U.S. Provisional Patent Application No. 60/955,001, filed on Aug. 9, 2007; U.S. Provisional Patent Application No. 60/956,468, filed Aug. 17, 2007; and U.S. Provisional Patent Application No. 60/978,298 filed Oct. 8, 2007, each of which is incorporated herein by reference in its entirety for all purposes.

### FIELD OF THE INVENTION

The invention pertains to the field of polypeptide manufacturing. In particular, the invention provides methods for the manufacturing glycosylated polypeptides using a baculoviral expression system.

### BACKGROUND OF THE INVENTION

With the development and refinement of recombinant-DNA techniques, it was anticipated that large-scale production of therapeutic polypeptides could be achieved in a cost effective manner using genetically modified bacteria. However, many heterologous proteins produced in E. coli are insoluble and difficult to purify. Furthermore, the majority of therapeutic proteins require post-translational modifications, such as glycosylation to become biologically active. Bacterial cells are often not suitable to provide polypeptides with desirable post-translational modifications.

Proper glycosylation is a critical factor influencing the in vivo half life and immunogenicity of therapeutic polypeptides. Typically, humans tolerate only those biotherapeutics that incorporate particular types of carbohydrate residues and will often reject glycoproteins that include non-mammalian oligosaccharides. For instance, poorly glycosylated polypeptides are recognized by the liver as being “old” and thus, are more quickly eliminated from the body than are properly glycosylated peptides. In contrast, hyperglycosylated peptides or incorrectly glycosylated peptides can be immunogenic. Since all mammals produce glycans of similar structure and in order to meet the requirements for proper glycosylation, mammalian cells are often chosen to produce therapeutic glycoproteins. Chinese Hamster Ovary (CHO), Baby Hamster Kidney (BHK) and Human Embryonic Kidney-293 (HEK-293) cells are among the preferred host cells for the production of glycoprotein therapeutics.

However, mammalian cell cultures are typically characterized by low cell densities and low growth rates. Furthermore, maintenance and growth of mammalian cell cultures can be cost-intensive and gene manipulations are difficult. In addition, mammalian cell have the potential for containing oncogenes or viral DNA that can affect human subjects. Therefore, recombinant polypeptides produced in mammalian cells require extensive safety testing.

To overcome the problems associated with polypeptide production in mammalian cells, insect cell culture systems have been developed. Insect cells possess metabolic pathways for processing glycoproteins that are similar to those of mammalian cells. Thus, insect cells in combination with a suitable expression system, such as the baculovirus expression vector system (BEVS), are most useful for the production of recombinant glycoproteins.

The BEVS has several advantages as a recombinant protein production system. For example, the time from gene isolation to expression can be as short as 4-6 weeks. Production levels are typically higher than those achievable using mammalian cell lines, and adventitious viruses (commonly found in mammalian tissue culture cells) are typically absent. Importantly, insect cells are able to recognize the co- and post-translational signals of higher eukaryotes, effecting intracellular processes, such as phosphorylation, proteolysis, carboxylmethylation, and glycosylation.

Given the many advantages of the BEVS over mammalian expression systems for the production of recombinant glycoproteins, it is not surprising that interest in improving insect cell culture technology has increased in recent years (see e.g., Schlaeger E, Cytotechnology 1996, 20:57-70, for a review). In particular, purification processes are needed that are efficient in isolating polypeptides from a variety of insect-cell derived and baculoviral contaminants, such as proteolytic enzymes to provide high quality pharmaceutical products that are safe for use in humans. As will be apparent from the disclosure that follows, the present invention meets this, and other needs.

### SUMMARY OF THE INVENTION

The present invention provides methods for the production (e.g., large-scale production) of polypeptides and glycopeptides. Exemplary methods are useful for the rapid isolation of recombinant polypeptides from insect cell-culture liquids, which include degradative enzymes, such as endoglycanases and proteases. In a particular example, the polypeptide is isolated from such enzymes using anion exchange (O) chromatography or Q filtration. An exemplary anion exchange step involves the use of a mixed-mode chromatography medium that combines anion exchange capabilities with hydrophobic interaction and/or hydrogen-bonding capabilities. Minimizing enzymatic degradation early in the process significantly improves overall recovery of active polypeptide and thus reduces manufacturing costs. In one embodiment, the polypeptide solution produced by a method of the invention is essentially free of endoglycanase and proteolytic activities. In another embodiment, the polypeptide is enriched to about 30% purity.

Another advantage of the current process is that it reduces the number of processing steps and the time that is needed to process a culture liquid from intial harvest through the first polypeptide capture step. Rapid processing early in the purification process is important because it minimizes the time that the polypeptide is exposed to degradation. An exemplary method of the invention requires less than 2 hours to process an insect-cell culture from harvest through initial polypeptide capture with an overall polypeptide recovery of about 70%. This can be accomplished by connecting early processing steps into single-unit operations and by selecting filtration and chromatography media suitable for rapid processing of insect cell-culture media. The efficient combination of early purification steps also minimizes protein precipitation, which, in turn, prevents fouling of downstream equipment and loss of polypeptide.

In one embodiment, the invention provides a method of isolating a recombinant polypeptide from an insect cell-culture using mixed-mode chromatography or mixed-mode filtration. The resulting partially purified polypeptide solution is essentially free of endoglycanase activity. In another embodiment, the partially purified polypeptide solution after mixed-mode chromatography is characterized by very low residual proteolytic activity (e.g., less than 3%).

In yet another embodiment, the method includes mixed-mode chromatography in combination with dye-ligand affinity chromatography. For example, after the cell culture liquid is filtered to remove cellular debris and other particles (e.g., using hollow fiber filtration, optionally followed by diafiltration), the pre-cleared solution is subjected to a combination of mixed-mode filtration and dye-ligand affinity chromatography, wherein the latter is useful to capture the desired polypeptide. The two purification steps may be arranged in a continuous-flow processing module by connecting the two media so that the flow-through from the mixed-mode filtration step is not collected but enters the dye-ligand affinity column directly upon elution.

Hence, in one aspect, the invention provides a method of making a composition that includes a recombinant polypeptide, wherein the polypeptide is expressed in an insect cell (e.g., using a baculoviral expression system) and wherein the composition is essentially free of endoglycanase activity. The method includes: (a) subjecting a mixture including the polypeptide to mixed-mode chromatography including the steps of: (i) contacting the mixture and a mixed-mode chromatography medium; and (ii) eluting the polypeptide from the mixed-mode chromatography medium generating a flow-through fraction comprising the polypeptide. In one embodiment, the mixed-mode chromatography medium is an anion exchanger including a mixed-mode ligand incorporating a quaternary amino group. In another embodiment, the mixed-mode ligand includes a hydrophobic moiety, such as a phenyl substituent, in addition to the quaternary amino group. In yet another embodiment, the mixed-mode ligand includes a moiety incorporating at least one hydroxyl group or another substituent providing hydrogen-bonding capabilities, in addition to the quaternary amino group. An exemplary mixed-mode chromatography medium useful in the methods of the invention is Capto Adhere.

The above described method may further include: (b) subjecting the flow-though fraction from the mixed-mode filtration step to dye-ligand affinity chromatography by contacting the flow-through fraction with a dye-ligand affinity chromatography medium under conditions sufficient for the polypeptide to reversibly bind the dye-ligand affinity chromatography medium; and eluting the polypeptide from the dye-ligand affinity chromatography medium generating an eluate fraction containing the polypeptide. In one example, the dye-ligand affinity medium is Capto Blue.

In another aspect, the invention provides a method of making a composition including a recombinant polypeptide of the invention, wherein the composition is essentially free of endoglycanase activity and essentially free of proteolytic activity. The method includes: (a) eluting a mixture including the polypeptide from a mixed-mode chromatography medium comprising a mixed-mode ligand having a quaternary amino group and at least one moiety selected from a hydrophobic moiety and a moiety comprising a hydroxyl group, thereby generating a flow-through fraction comprising the polypeptide; (b) contacting the flow-through fraction with a dye-ligand affinity chromatography medium; and (c) eluting the polypeptide from the dye-ligand affinity chromatography medium, thereby producing an eluate fraction including the polypeptide. The method may further include: irradiating the eluate fraction of step (c) with UV light in a manner sufficient to effect viral inactivation.

In one example according to any of the above embodiments, the residual endoglycanase activity of the eluate fraction from the dye-ligand affinity step is less than about 1% and preferably less than about 0.5% compared to the endoglycanase activity of the mixture prior to mixed-mode chromatography and dye-ligand affinity chromatography. In another example, the eluate fraction has a residual proteolytic activity that is less than about 5%, preferably less than 3% and more preferably less than 2% of the proteolytic activity prior to mixed-mode chromatography and dye-ligand affinity chromatography. In yet another example, the polypeptide after mixed-mode chromatography and dye-ligand affinity chromatography has a purity of at least about 25% and preferably of at least about 30% (w/w). In a further example, at least 60%, preferably at least 65% and more preferably at least 70% of the polypeptide that is loaded onto the mixed-mode medium is recovered in the eluate fraction of the dye-ligand affinity chromatography step.

Any of the above described methods may further include: eluting the polypeptide from at least one, preferably two different chromatography media. Each chromatography medium is selected from a hydrophobic interaction chromatography medium, a cation exchange chromatography medium, an anion exchange chromatography medium and a hydroxyapatite or fluoroapatite chromatography medium. In one embodiment, the polypeptide is eluted from a mixed-mode filter and a dye-ligand affinity resin before it is subjected to hydrophobic interaction chromatography and cation exchange chromatography.

An exemplary method according to any of the above embodiments, further includes: infecting insect cells (e.g., Spodoptera frugiperda cells) in an insect cell culture with a recombinant baculovirus comprising a nucleotide sequence encoding the polypeptide. In one embodiment, the insect cells are infected with the baculovirus in a cell culture medium that is supplemented with a lipid mixture of the invention.

In one example, the polypeptide in any of the above discussed methods is erythropoietin (EPO).

In another example according to any of the above embodiments, the mixed-mode chromatography medium is a strong anion exchanger and includes, for example, a mixed-mode ligand having a quaternary amino group. The mixed-mode ligand may further provide hydrophobic interaction capabilities (e.g., through the presence of a hydrophobic moiety) and/or may also provide hydrogen-bonding capabilities (e.g., through the presence of a moiety that includes at least one hydroxyl group). An exemplary mixed-mode medium useful in the methods of the invention combines anion exchange capabilities with both hydrophobic interaction capabilities and hydrogen-bonding capabilities. One medium having those characteristics is Capto Adhere.

In another example according to any of the above embodiments, the dye-ligand affinity chromatography medium includes Cibacron Blue immobilized on a solid support, such as a sepharose- or an agarose-based matrix. An exemplary dye-ligand affinity medium useful in the methods of the invention is Capto Blue.

In one embodiment, the method of the invention may further include: removing cellular debris from a cell-culture liquid including the polypeptide. In one example, cellular debris is removed from the cell culture liquid using filtration, such as hollow fiber filtration. An exemplary polypeptide purification process, in which hollow fiber filtration of the cell culture liquid, mixed-mode chromatography (e.g., Capto Adhere) and dye-ligand affinity chromatography are connected in a single-unit operation is illustrated in FIG. 2.

The invention also provides compositions made by the methods of the invention. It further provides pharmaceutical formulations that include a composition of the invention and a pharmaceutically acceptable carrier. In addition, the invention provides methods of using the compositions and pharmaceutical formulations of the invention.

In some embodiments, the recombinant peptides produced by the methods of the invention are glycopeptides and are further processed to elaborate the structure of their glycosyl residues. In other embodiments the glycopeptides are used to create glycopeptide conjugates, in which the polypeptide is covalently linked to a modifying group, such as a polymer (e.g., polyethylene glycol). In one example, the method includes glycoPEGylating the isolated polypeptide. Glycopegylation methods are aft-recognized. See for example, WO 03/031464 to De Frees et al., and WO 04/99231 to De Frees et al., the disclosures of which are incorporated herein by reference in their entirety

In one embodiment, the method is used to produce a therapeutic peptide, such as erythropoietin (EPO) and granulocyte colony stimulating factor (GCSF). Alternatively, the method can be used to produce other recombinant peptides such as enzymes (e.g., GNT1, GalT1, ST3Gal3, GalNAcT2, Core1GalT, ST6GalNAc1, ST3Gal1 and ST3Gal2).

Other objects and advantages of the invention will be apparent to those of skill in the art from the detailed description that follows.

### DESCRIPTION OF THE DRAWINGS

FIG. 1 is an overall view of a polypeptide purification process according to an exemplary method of the invention. Early processing steps focus on the removal of cellular debris by filtration, removal of degradative enzymes by mixed-mode filtration, polypeptide capture using dye-ligand affinity chromatography or cation exchange chromatography, inactivation of potential viruses and removal of viral particles by membrane filtration. The partially purified polypeptide solution is then processed using a combination of chromatographic steps including hydrophobic interaction chromatography (HIC) and cation exchange chromatography. The process may further include hydroxyapatite or fluoroapatite chromatography. The polypeptide solution may then be filtered again, before the purified polypeptide is formulated into a storage buffer or used in subsequent processes.

FIG. 2 is an exemplary process flow diagram. The depicted process includes a process module, in which a mixed-mode medium (e.g., Capto Adhere) and a dye-ligand affinity medium (e.g., Capto Blue) are combined into a continuous process step. The exemplary flow diagram also illustrates a hollow fiber filtration step prior to mixed-mode and dye-ligand affinity chromatography.

FIG. 3 is a diagram outlining an exemplary method for the determination of endoglycanase activity in a partially purified polypeptide solution. Solid squares represent GlcNAc residues, open circles represent mannose residues and solid triangles represent fucose residues. In one example, the buffer of the test solution is exchanged using a membrane (e.g., 10 kDa MWCO) that allows for the removal of free glycans and other reducing sugars from the sample. Polypetide substrate is then added in excess and the mixture is incubated for about 18-22 hours at 30-37° C. Cleaved glycans are isolated from the polypeptide by filtration. The reducing ends of the glycans are reacted with a detection reagent to produce a detectable label (e.g., fluorescent label). Labeled glycans are analyzed using HPLC. Endoglycanase activity may be determined as the ratio between the signal produced by an internal standard and the signal produced by the test sample.

FIG. 4 is a graph illustrating the pH dependency of endoglycanase activity. The experiment was performed using endoH in various buffer systems and a glycosylated protein as the substrate. The Y-axis depicts relative endoglycanase activity, wherein the activity at pH 6 (approximate pH maximum) was set at 100%. The graph is a result of three independent experiments. Endoglycanase activity was determined using the assay illustrated in FIG. 3 and outlined in Example 1.

FIG. 5 is a diagram illustrating the effect of various additives and conditions on endoglycanase activity in a buffer containing 25-40 mM MES, 25-40 mM NaCl at pH 6, unless otherwise indicated. The Y-axis depicts relative endoglycanase activity as compared to a control activity (no additive, 100%). The identities of the samples are: (1) 25 mM cibacron blue; (2) 0.8 M KCl; (3) 1.6 M KCl; (4) 1.6 M KCl at pH 8.5; (5) 1.5% lipid mix of the invention; (6) 4° C.; (7) 20 mM caffeine; (8) riboflavin (0.7 mM); (9) 0.6 M guanidine HCl; (10) 50 mM MgCl₂; (11) 50 mM ZnCl₂; (12) 10 mM CaCl₂ at pH 7.5; (13) 10 mMEDTA at pH 7.5.

FIG. 6 is an elution profile obtained by processing a 15 L pre-cleared insect cell culture sample using a Capto Adhere/Capto Blue continuous process module as described in Example 3. The purified polypeptide (EPO) is found in the flow-through of the Capto Adhere filter and is subsequently captured by the Capto Blue resin. The elution profile illustrates the elution of impurities that are unbound or weakly bound by the Capto Blue medium as well as the elution of EPO after disconnection of the Capto Adhere column from the module using a buffer containing 2 M KCl. Two EPO containing fractions, labeled 1 and 2, were collected and analyzed separately.

### DETAILED DESCRIPTION OF THE INVENTION

### Abbreviations

PEG, poly(ethyleneglycol); PPG, polytpropyleneglycol); Ara, arabinosyl; Fru, fructosyl; Fuc, fucosyl; Gal, galactosyl; GalNAc, N-acetylgalactosaminyl; Glc, glucosyl; GicNAc, N-acetylglucosaminyl; Man, mannosyl; ManAc, mannosaminyl acetate; Xyl, xylosyl; and NeuAc, sialyl (N-acetylneuraminyl); M6P, mannose-6-phosphate; BEVS, baculovirus expression vector system; CV, column volume; NTU, nominal turbidity units; vvm, volume/volume/min; ACN, acetonitrile; mcL, microliter.

### DEFINITIONS

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry and nucleic acid chemistry and hybridization are those well known and commonly employed in the art. Standard techniques are used for nucleic acid and peptide synthesis. The techniques and procedures are generally performed according to conventional methods in the art and various general references (see generally, Sambrook et al. MOLECULAR CLONING: A LABORATORY MANUAL, 2d ed. (1989) Cold Spring Harbor Laboratory Press, Cold Spring Harbor, N.Y., which is incorporated herein by reference), which are provided throughout this document. The nomenclature used herein and the laboratory procedures in analytical chemistry, and organic synthetic described below are those well known and commonly employed in the art. Standard techniques, or modifications thereof, are used for chemical syntheses and chemical analyses.

Where substituent groups are specified by their conventional chemical formulae, written from left to right, they equally encompass the chemically identical substituents, which would result from writing the structure from right to left, e.g., —CH₂O— is intended to also recite —OCH₂—.

The term “alkyl” by itself or as part of another substituent, means, unless otherwise stated, a straight or branched chain, or cyclic (i.e., cycloalkyl)hydrocarbon radical, or combination thereof, which may be fully saturated, mono- or polyunsaturated and can include di- (e.g., alkylene) and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below, such as “heteroalkyl.” Alkyl groups that are limited to hydrocarbon groups are termed “homoalkyl”.

The term “alkylene” by itself or as part of another substituent means a divalent radical derived from an alkane, as exemplified, but not limited, by —CH₂CH₂CH₂CH₂—, and further includes those groups described below as “heteroalkylene.” Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom selected from the group consisting of O, N, Si and S, and wherein the nitrogen and sulfur atoms may optionally be oxidized and the nitrogen heteroatom may optionally be quaternized. The heteroatom(s) O, N and S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —CO₂R′— represents both —C(O)OR′ and —OC(O)R′.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

The term “aryl” means, unless otherwise stated, a polyunsaturated, aromatic, substituent that can be a single ring or multiple rings (preferably from 1 to 3 rings), which are fused together or linked covalently. The term “heteroaryl” refers to aryl groups (or rings) that contain from one to four heteroatoms selected from N, O, S, Si and B, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) are meant to include both substituted and unsubstituted forms of the indicated radical unless otherwise indicated. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) are generically referred to as “alkyl group substituents,” and they can be one or more of a variety of groups selected from, but not limited to: substituted or unsubstituted aryl, substituted or unsubstituted heteroaryl, substituted or unsubstituted heterocycloalkyl, —OR′, ═O, ═NR′, ═N—OR′, —NR′R″, —SR′, -halogen, —SiR′R″R″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R″)═NR″″, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical. R′, R″, R′″ and R″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are generically referred to as “aryl group substituents.” The substituents are selected from, for example: substituted or unsubstituted alkyl, substituted or unsubstituted heteroalkyl, substituted or unsubstituted aryl, substituted or unsubstituted heteroaryl, substituted or unsubstituted heterocycloalkyl, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″ and R″″ are preferably independently selected from hydrogen, substituted or unsubstituted alkyl, substituted or unsubstituted heteroalkyl, substituted or unsubstituted aryl and substituted or unsubstituted heteroaryl. When a compound of the invention includes more than one R group, for example, each of the R groups is independently selected as are each R′, R″, R′″ and R″″ groups when more than one of these groups is present.

Two of the substituents on adjacent atoms of the aryl or heteroaryl ring may optionally be replaced with a substituent of the formula -T-C(O)—(CRR′)_{q}-U-, wherein T and U are independently —NR—, —O—, —CRR′— or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X—(CR″R′″)_{d}—, where s and d are independently integers of from 0 to 3, and X is —O—, —NR′—, —S—, —S(O)—, —S(O)₂—, or —S(O)₂NR′—. The substituents R, R′, R″ and R′″ are preferably independently selected from hydrogen or substituted or unsubstituted (C₁-C₆)alkyl.

As used herein, the term “heteroatom” includes oxygen (O), nitrogen (N), sulfur (S), silicon (Si) and boron (B).

The symbol “R” is a general abbreviation that represents a substituent group. Exemplary substituent groups include substituted or unsubstituted alkyl, substituted or unsubstituted heteroalkyl, substituted or unsubstituted aryl, substituted or unsubstituted heteroaryl, and substituted or unsubstituted heterocycloalkyl groups.

All oligosaccharides described herein are described with the name or abbreviation for the non-reducing saccharide (i.e., Gal), followed by the configuration of the glycosidic bond (α or β), the ring bond (1 or 2), the ring position of the reducing saccharide involved in the bond (2, 3, 4, 6 or 8), and then the name or abbreviation of the reducing saccharide (i.e., GlcNAc). Each saccharide is preferably a pyranose. For a review of standard glycobiology nomenclature see, Essentials of Glycobiology Varki et al. eds. CSHL Press (1999).

Oligosaccharides are considered to have a reducing end and a non-reducing end, whether or not the saccharide at the reducing end is in fact a reducing sugar. In accordance with accepted nomenclature, oligosaccharides are depicted herein with the non-reducing end on the left and the reducing end on the right.

The term “insect cell culture” refers to the in vitro growth and culturing of cell derived from organisms of the Class Insecta. “Insect cell culture” also refers to a cell culture comprising cells of the Class Insecta which have been grown and cultured in vitro.

The term “multiplicity of infection” refers to a measure of the ratio between the number of viral particles and the number of cells to be infected by the viral particles, e.g., number of plaque forming units (pfu) per cell, or viral particles per cell. The efficiency of infection is influenced by the MOI as well as by the concentration of viral particles and the concentration of cells.

The multiplicity of infection is also a reflection of the average number of viral particles infecting each cell when the cells and viral particles are mixed in order to initiate infection. Indeed, the number of viral particles binding to and infecting any given cell is a random process, therefore there is statistical variation in the number of particles that bind to and infect each cell. The statistical variation follows a normal distribution. Thus, most cells will be infected with a number of virus particles corresponding to the MOI. However, some cells will be infected by more or fewer particles, and some will be infected by no particles at all. The number of cells escaping infection can be calculated using the Poisson distribution. According to the Poisson distribution, the number of cells remaining uninfected at any given MOI is e^{-MOI}.

“Peptide” refers to a polymer in which the monomers are amino acids and are joined together through amide bonds, alternatively referred to as a polypeptide. Additionally, unnatural amino acids, for example, β-alanine, phenylglycine and homoarginine are also included. Amino acids that are not gene-encoded may also be used in the present invention. Furthermore, amino acids that have been modified to include reactive groups, glycosylation sites, polymers, therapeutic moieties, biomolecules and the like may also be used in the invention. All of the amino acids used in the present invention may be either the D- or L-isomer. The L-isomer is generally preferred. In addition, other peptidomimetics are also useful in the present invention. As used herein, “peptide” refers to both glycosylated and unglycosylated peptides. Also included are peptides that are incompletely glycosylated by a system that expresses the peptide. For a general review, see, Spatola, A. F., in CHEMISTRY AND BIOCHEMISTRY OF AMINO ACIDS, PEPTIDES AND PROTEINS, B. Weinstein, eds., Marcel Dekker, New York, p. 267 (1983). The term peptide includes molecules that are commonly referred to as proteins or polypeptides.

A “glycopeptide” as the term is used herein refers to a peptide having at least one carbohydrate moiety covalently linked thereto. It is understood that a glycopeptide may be a “therapeutic glycopeptide”. The term “glycopeptide” is used interchangeably herein with the terms “glycopolypeptide” and “glycoprotein.”

The term “peptide conjugate” refers to species of the invention in which a peptide is conjugated with a modified sugar as set forth in, e.g., WO 03/031464 to De Frees et al., which is incorporated herein by reference in its entirety.

As used herein, the term “modified sugar” refers to a naturally- or non-naturally-occurring carbohydrate that is enzymatically added onto an amino acid or a glycosyl residue of a peptide in a process of the invention. The modified sugar is selected from a number of enzyme substrates including, but not limited to sugar nucleotides (mono-, di-, and tri-phosphates), activated sugars (e.g., glycosyl halides, glycosyl mesylates) and sugars that are neither activated nor nucleotides. The “modified sugar” is covalently functionalized with a “modifying group.” Useful modifying groups include, but are not limited to, PEG moieties, therapeutic moieties, diagnostic moieties, biomolecules and the like. The modifying group is preferably not a naturally occurring, or an unmodified carbohydrate. The locus of functionalization with the modifying group is selected such that it does not prevent the “modified sugar” from being added enzymatically to a peptide.

The term “glycoconjugation” as used herein, refers to the enzymatically mediated conjugation of a modified sugar species to an amino acid or glycosyl residue of a polypeptide, e.g., an erythropoietin peptide prepared by the method of the present invention. A subgenus of “glycoconjugation” is “glyco-PEGylation,” in which the modifying group of the modified sugar is poly(ethylene glycol), an alkyl derivative (e.g., m-PEG) or reactive derivative (e.g., H₂N-PEG, HOOC-PEG) thereof.

The terms “large-scale” and “industrial-scale” are used interchangeably and refer to a reaction cycle or process that produces at least about 250 mg, preferably at least about 500 mg, and more preferably at least about 1 gram of peptide at the completion of a single cycle.

The term, “glycosyl linking group” as used herein refers to a glycosyl residue to which a modifying group (e.g., PEG moiety, therapeutic moiety, biomolecule) is covalently attached; the glycosyl linking group joins the modifying group to the remainder of the conjugate. A “glycosyl linking group” is generally formed by the enzymatic addition of a modified sugar moiety to a glycosyl residue or amino acid of a peptide.

The term “isolated” or “purified” when referring to a polyeptide or polypeptide solution of the invention, means that such material is essentially free from components, which are used to produce the material. For polypeptides of the invention, the term “isolated” refers to a material that is essentially free from components which normally accompany the material in the mixture used to prepare the polypeptide (e.g., cellular proteins derived from the host cell). “Isolated”, “pure” or “purified” are used interchangeably. Purity can be determined by any art-recognized method of analysis (e.g., band intensity on a silver stained gel, polyacrylamide gel electrophoresis, HPLC, ELISA, or a similar means). In one example, purity is determined as the ratio between the amount of desired polypeptide and the amount of total polypeptide/protein present in a sample (w/w). For example the concentration of the polypeptide in the sample may be determined using analytical chromatography (e.g., HPLC, RP-HPLC) in combination with a protein standard. Total protein content in a sample may be determined using a standard protein assay (e.g., Bradford), such as those based on absorbance at a particular wave-length (e.g., A280). Purity of the polypeptide of interest is then determined by calculating the ratio between the two values obtained.

Typically, polypeptides isolated using a method of the invention, have a level of purity expressed as a range. The lower end of the range of purity for the polypeptide is about 30%, about 40%, about 50%, about 60%, about 70%, about 75% or about 80% and the upper end of the range of purity is about 70%, about 75% about 80%, about 85%, about 90%, about 95% or more than about 95%.

When the polypeptide is more than about 90% pure, its purity is also preferably expressed as a range. The lower end of the range of purity is about 90%, about 92%, about 94%, about 96% or about 98%. The upper end of the range of purity is about 92%, about 94%, about 96%, about 98% or about 100% purity.

“Polypeptide recovery” or “yield” is typically expressed as the range between the amount of recovered polypeptide after a particular process step (or series of steps) and the amount of polypeptide that entered the process step. For example, the recovery of polypeptide for a method of the invention is about 20%, about 30%, about 40%, about 50%, about 60%, about 70%, about 80% or about 90%. In another example, the polypeptide recovery for a method of the invention is about 92%, about 94%, about 96%, about 98% or more than about 98%.

The term “mixed-mode ligand” refers to a molecule covalently linked to a solid support/matrix of a mixed-mode chromatography medium.

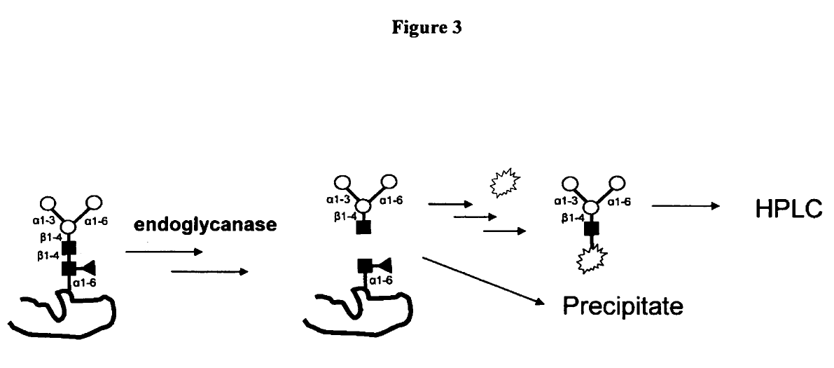

The term “endoglycanase” is used interchangably with the term 4“endoglycosidase” and refers to an enzyme, which is capable of cleaving a glycosyl moiety off a glycan residue of a polypeptide (e.g., EPO). An exemplary reaction catalyzed by an endoglycanase is illustrated in FIG. 3.

“Essentially free of endoglycanase activity” refers to a purified or partially purified polypeptide solution that does not contain endoglycanases or that contains a very low concentration of endoglycanases. In this application endoglycanase activity is typically reported as a ratio between the activity detected after and the activity detected prior to applying a method of the invention and can be understood as “residual endoglycanase activity”. For example, a polypeptide solution having endoglycanase activity is subjected to mixed-mode chromatography thereby reducing the endocglycanase activity in the polypeptide solution to 20% of the original activity. Thus, the residual endoglycanase activity after mixed-mode chromatography is 20%. In one embodiment, “essentially free of endoglycanase activity” means that the residual endoglycanase activity after applying a method of the invention is less than about 50%, preferably less than about 40%, less than about 35%, less than about 30%, less than about 25% or less than about 20%. In another embodiment, the residual endoglycanase activity is less than about 15%, preferably less than about 10%, less than about 9%, less than about 8%, less than about 7%, less than about 6% or less than about 5%. In another embodiment, the residual endoglycanase activity is less than about 4%, less than about 3%, less than about 2% or less than about 1%. In yet another embodiment, “essentially free of endoglycanase activity” means that the activity is reduced to less than about 0.5%, less than about 0.4%, less than about 0.3%, less than about 0.2% or less than about 0.1%. In a particularly preferred embodiment, the endoglycanase activity is reduced to less than about 0.08%, less than about 0.06%, less than about 0.04% or less than about 0.02%. Assay formats useful for the determination of endoglycanase activity are know to those of skill in the art. An exemplary method is described herein in Example 1 and illustrated in FIG. 3.

The term “protease” is used herein according to its art recognized meaning and refers to an enzyme that exhibits proteolytic activity, meaning that it can cleave a polypeptide chain, thereby generating polypeptide fragments.

“Essentially free of proteolytic (or protease) activity” refers to a purified or partially purified polypeptide solution. In this application proteolytic activity is typically reported as a ratio between the activity detected after and the activity detected prior to applying a method of the invention. For example, a polypeptide solution having proteolytic activity is subjected to mixed-mode chromatography thereby reducing the proteolytic activity in the polypeptide solution to 20% of the original activity. Thus the ratio between the proteolytic activities after and before the mixed-mode chromatography is 20%. In one embodiment, “essentially free of proteolytic activity” means that the ratio between the proteolytic activities after and before applying a method of the invention is less than about 50%, preferably less than about 40%, less than about 35%, less than about 30%, less than about 25% or less than about 20%. In another embodiment, the ratio is less than about 15%, preferably less than about 10%, less than about 9%, less than about 8%, less than about 7%, less than about 6% or less than about 5%. In another embodiment, the proteolytic activity is reduced to less than about 4%, less than about 3%, less than about 2%, less than about 1.5% or less than about 1% of the original activity. Assay formats to determine protease/proteolytic activity are know to those of skill in the rat. An exemplary method is described herein (Example 2).

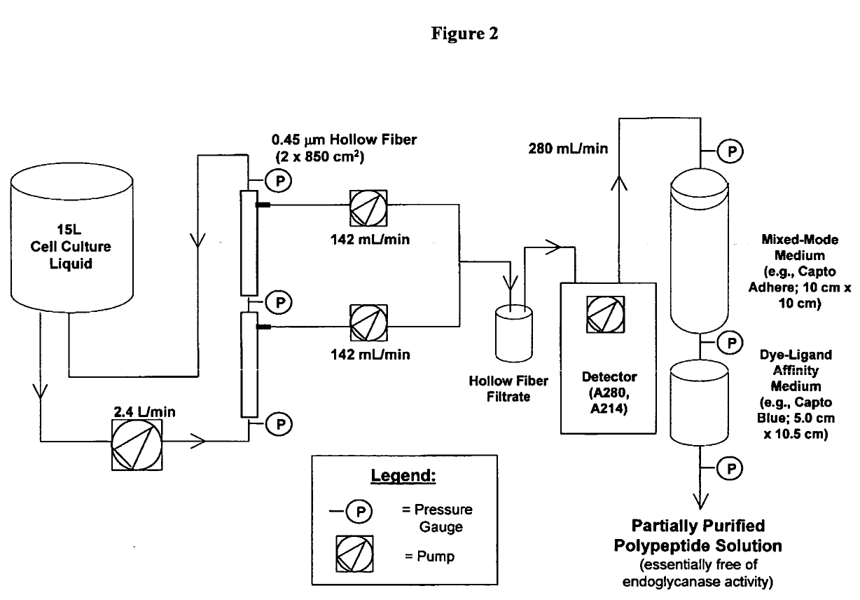

“Essentially immediately after elution” refers to a first chromatography/filtration step, in which the polypeptide elutes from a first chromatography medium (e.g., mixed-mode or anion exchange medium) and is then contacted with a second chromatography medium (e.g., a dye-ligand affinity medium). “Essentially immediately after elution” means that the time between elution from the first medium and contact with the second medium is not more than about 6 hours, preferably not more than about 5 hours, more preferably not more than about 4 hours and most preferably not more than about 3 hours. In one embodiment, the time between elution from the first medium and contact with the second medium is not more than about 2 hours or not more than about 1 hour. In a particularly preferred embodiment, “essentially immediately after elution” means that the two chromatography/filtration steps are linked in a continuous flow process module. In this embodiment, the eluate from the first step is not collected but is contacted with the second medium directly upon elution from the first medium. An exemplary process module including a mixed-mode medium and a dye-ligand affinity medium is depicted in FIG. 2.

“Essentially each member of the population” as used herein, speaks to the “homogeneity” of the sites on the peptide and to a population of peptide that share a common structure, e.g., a common glycosyl structure.

“Homogeneity” refers to the structural consistency across a population of peptides or across a population of glycosylation site on a peptide. Thus, in a glycopeptide of the invention in which each glycosyl moiety has the same structure the glycopeptide is said to be about 100% homogeneous. Similarly, when a population of glycopeptides of the invention all have glycosyl moieties of the same structure, such that each peptide of the population is essentially of the same molecular species, the population is said to be about 100% homogeneous. Homogeneity is typically expressed as a range. The lower end of the range of homogeneity for the peptide conjugates is about 60%, about 70% or about 80% and the upper end of the range of purity is about 70%, about 80%, about 90% or more than about 90%.

When the peptide conjugates are more than or equal to about 90% homogeneous, their homogeneity is also preferably expressed as a range. The lower end of the range of homogeneity is about 90%, about 92%, about 94%, about 96% or about 98%. The upper end of the range of purity is about 92%, about 94%, about 96%, about 98% or about 100% homogeneity. The homogeneity of the peptide conjugates is typically determined by one or more methods known to those of skill in the art, e.g., gel electrophoresis, liquid chromatography-mass spectrometry (LC-MS), matrix assisted laser desorption mass time of flight spectrometry (MALDITOF), capillary electrophoresis, and the like.

“Substantially uniform glycosylation pattern,” when referring to a glycopeptide species of the invention, refers to the percentage of glycosylation sites on the peptide that have a glycosyl residue of the same structure. For example a peptide that includes multiple glycosylation site may have a glycosyl residue of the same structure present at all of the possible glycosylation sites or even at 90% of the sites or 80% or 75% of the sites. In these instances the peptide would be said to have a “substantially uniform glycosylation pattern”. Alternatively, when a population of glycopeptides share a common glycosylation pattern, the population may be said to have a “substantially uniform glycosylation pattern” when a majority of the peptides in the population represent essentially a single molecular species.

For instance, when glycosylated peptides are isolated from a cell, without further modification, the peptides may include a range of variations in the precise structure of the glycan. However, in an exemplary embodiment, peptides isolated from insect cells according to the method of the invention have a substantially uniform insect glycosylation pattern. This refers to the fact that the majority of peptides, or substantially all of the peptides, in the preparation represent one distinct molecular species. In an exemplary embodiment, a peptide prepared by the method of the invention has a substantially uniform insect glycosylation pattern.

The term “substantially” in the above definitions of “substantially uniform” generally means at least about 40%, at least about 45%, at least about 50%, at least about 55%, at least about 60%, at least about 65%, at least about 70%, 71%, 72%, 73%, 74%, 75%, 76%, 77%, 78%, 79%, at least about 80%, 81%, 82%, 83%, 84%, 85%, 86%, 87%, 88%, 89%, at least about 90%, 91%, 92%, 93%, 94%, 95%, 96%, 97%, 98%, 99%, or even 100% of the acceptor moieties are glycosylated with the expected insect cell specific glycosylation pattern.

The term “Insect specific glycosylation pattern” refers to the glycosylation pattern found on mature glycopeptides produced by insect cells. Typically insect cells generate simple N-linked oligosaccharides terminating in mannose (for review, see e.g., Essentials of Glycobiology A. Varki er a. eds, CSHL Press (1999) pgs: 32-33). Typically, N-linked glycans produced by insect cell lines produce glycoproteins that at maturity, include a Man₃GlcNAC₂ structure. Fucose units may also be found on the GlcNAc residue that is directly linked to the peptide. A mature peptide emerging from a cell with an “insect specific glycosylation pattern” thus includes one or more glycans having the Man₃GlcNAc₂ structure.

The term “loading buffer” refers to the buffer, in which the peptide being purified is applied to a purification device, e.g. a chromatography column or a filter cartridge. Typically, the loading buffer is selected so that separation of the peptide of interest from unwanted impurities can be accomplished. For instance, when purifying the peptide on a hydroxyapatite (HA) or fluoroapatite column the pH of the loading buffer and the salt concentration in the loading buffer may be selected so that the peptide is initially retained on the column while certain impurities are found in the flow through.

The term “elution buffer”, also called “limit buffer”, refers to the buffer, which is typically used to remove (elute) the peptide from the purification device (e.g. a chromatographic column or filter cartridge) to which it was applied earlier. Typically, the loading buffer is selected so that separation of the peptide of interest from unwanted impurities can be accomplished. Often the concentration of a particular salt (e.g. NaCd) in the elution buffer is varied during the elution procedure (gradient). The gradient may be continuous or stepwise.

The term “controlled room temperature” refers to a temperature of at least about 10° C., at least about 15° C., at least about 20° C. or at least about 25° C. Typically, controlled room temperature is between about 20° C. and about 25° C.

The term “chromatography” includes the term “filtration”.

As used herein, “pharmaceutically acceptable carrier” includes any material, which when combined with the conjugate retains the conjugates' activity and is non-reactive with the subjects immune systems. “Pharmaceutically acceptable carrier” includes solids and liquids, such as vehicles, diluents and solvents. Examples include, but are not limited to, any of the standard pharmaceutical carriers such as a phosphate buffered saline solution, water, emulsions such as oil/water emulsion, and various types of wetting agents. Other carriers may also include sterile solutions, tablets including coated tablets and capsules. Typically such carriers contain excipients such as starch, milk, sugar, certain types of clay, gelatin, stearic acid or salts thereof, magnesium or calcium stearate, talc, vegetable fats or oils, gums, glycols, or other known excipients. Such carriers may also include flavor and color additives or other ingredients. Compositions comprising such carriers are formulated by well known conventional methods.

As used herein, “administering” means oral administration, administration as a suppository, topical contact, intravenous, intraperitoneal, intramuscular, intralesional, or subcutaneous administration, administration by inhalation, or the implantation of a slow-release device, e.g., a mini-osmotic pump, to the subject. Administration is by any route including parenteral and transmucosal (e.g., oral, nasal, vaginal, rectal, or transdermal), particularly by inhalation. Parenteral administration includes, e.g., intravenous, intramuscular, intra-arteriole, intradermal, subcutaneous, intraperitoneal, intraventricular, and intracranial. Moreover, where injection is to treat a tumor, e.g., induce apoptosis, administration may be directly to the tumor and/or into tissues surrounding the tumor. Other modes of delivery include, but are not limited to, the use of liposomal formulations, intravenous infusion, transdermal patches, etc.

The term “ameliorating” or “ameliorate” refers to any indicia of success in the treatment of a pathology or condition, including any objective or subjective parameter such as abatement, remission or diminishing of symptoms or an improvement in a patients physical or mental well-being. Amelioration of symptoms can be based on objective or subjective parameters; including the results of a physical examination and/or a psychiatric evaluation.

The term “therapy” refers to “treating” or “treatment” of a disease or condition including preventing the disease or condition from occurring in a subject (e.g., human) that may be predisposed to the disease but does not yet experience or exhibit symptoms of the disease (prophylactic treatment), inhibiting the disease (slowing or arresting its development), providing relief from the symptoms or side-effects of the disease (including palliative treatment), and relieving the disease (causing regression of the disease).

The term “effective amount” or “an amount effective to” or a “therapeutically effective amount” or any gramatically equivalent term means the amount that, when administered to an animal or human for treating a disease, is sufficient to effect treatment for that disease.

### Introduction

The large-scale production of recombinant polypeptides (e.g., EPO) using insect cells as the host cells (e.g., in combination with a baculoviral expression system) is associated with a variety of difficulties. One problem involves enzymatic degradation of the desired polypeptide through enzymes, such as proteases and endoglycanases (endoglycosidases). For example, insect-cell and/or baculoviral proteases contained in the cell-culture broth can lead to significant loss of polypeptide. Exemplary proteases that can cleave the polypeptide of interest include cysteine proteases, metalloproteases and aspartate proteases.

In addition, the presence of endoglycanases (endoglycosidases), which can alter a glycopeptide's glycan structure (e.g., cleavage of terminal glycosyl moieties), may result in the formation of undesired glycoforms of the purified polypeptide. Such degradation can negatively effect overall process yields. It is therefore highly desirable to remove both, protease and endoglycanase activities from the polypeptide solution early in the purification process in order to minimize polypeptide loss due to enzymatic degradation.

Another problem involves precipitation of the desired polypeptide and/or other proteins from the crude feed stream (e.g., cell culture broth) prior to capturing the polypeptide of interest (e.g., during pH changes, sample filtration, sample concentration, hold times and the like). Such precipitation can cause not only a significant reduction of the overall process yield, but can also lead to “membrane/column fouling” (i.e., clogging of purification media) when precipitation occurs during sample processing. It is thus desirable to minimize manipulations of the feed stream prior to the capture step and thereby discourage any protein/polypeptide precipitation.

EPO as an exemplary polypeptide is produced as a secreted polypeptide at approximately 20 mg/L by a baculovirus-infected Sf9 insect cell fermentation culture. In order to produce sufficient polypeptide quantities, it is beneficial to process at least about 1000 to about 5000 L fermentation volumes. Thus an efficient cell clarification and protein capture process is essential to concentrate the polypeptide solution to a manageable working volume suitable for downstream processing. The clarification and capture conditions, therefore, had three important requirements in order to maximize the yield of active polypeptide (e.g., tri-mannosyl core EPO). First, the process must be fast in order to minimize the exposure time of the polypeptide to the degradative enzyme activities present in the cell culture and to avoid precipitation of the polypeptide from the crude feed stream. Ideally, the clarification and capture processing steps should take no longer than about 1-2 hours even when scaled to 5000 L fermentation culture. Second, the capture step should be suitable to remove degradative enzyme activities (proteolysis and deglycosylation) while concentrating the polypeptide. Third, the polypeptide capture pool is preferably compatible with a viral kill step and downstream purification processes, preferably without requiring major dilution and/or ultrafiltration for buffer exchange.

Hence, in one aspect, the invention provides a method of capturing a recombinant polypeptide from an insect cell culture. The polypeptide capture step involves mixed-mode and dye-ligand affinity chromatography. The inventors have discovered that such capture step is useful to produce a polypeptide solution that is essentially free of endoglycanase and protease activities. The inventors have further discovered that the combination of mixed-mode and dye-ligand affinity chromatography can function as an effective capture step that requires minimal manipulation of the cell culture liquid prior to its application to the capture medium and thereby minimizes loss of polypeptide due to precipitation.

In another aspect the invention provides an infection procedure that provides cell culture liquids containing a recombinant polypeptide in high concentration and high purity. The inventors have discovered that infecting an insect cell culture with a recombinant baculovirus when a lipid mixture is present in the cell culture at the time of infection increases the amount of polypeptide expressed by the insect cells. In some embodiments, the amount of peptide in the cell culture is increased by about 80% when compared to the amount in a culture not supplemented with the lipid mixture. In other embodiments the amount of recombinant peptide in the cell culture is increased by about 40% when compared to the amount in a culture supplemented with a commercial lipid mixture.

The invention includes a newly discovered infection procedure that provides cell cultures containing a recombinant peptide in unexpectedly high concentration and purity. The present inventors have discovered that, contrary to the teachings of the prior art, infecting insect cells with a recombinant baculovirus when a lipid mixture is present in the cell culture at the time of infection, increases the amount of peptide expressed by the insect cells. In some embodiments, the amount of peptide in the cell culture is increased by about 82% when compared to the amount in a culture not supplemented with the lipid mixture. In other embodiments the amount of recombinant peptide in the cell culture is increased by about 38% when compared to the amount in a culture supplemented with a commercial lipid mixture. The method is particularly useful for large-scale production of glycopeptides.

An exemplary method of the invention, includes infecting insect cells in an insect cell culture with a recombinant baculovirus that includes a nucleotide sequence encoding a peptide. The infecting takes place in an insect cell culture that is supplemented with a lipid mixture. The infected insect cells are grown to produce the peptide encoded by the nucleic acid sequence. The peptide so produced has an insect-specific glycosylation pattern. In one embodiment, the peptide so produced has a substantially uniform, insect-specific glycosylation pattern.

In another embodiment, the method of the invention includes a viral inactivation step. In one embodiment the viral inactivation method includes lowering the pH of a peptide solution to a value suitable to decrease the viability of certain viruses (e.g. non-enveloped viruses) and maintaining this low pH (e.g. pH about 2.2) for a suitable amount of time (e.g. about 1 hour), before the pH is raised. The pH value and the holding period are selected to minimize degradation of the polypeptide while exposing it to the low-pH. In some embodiments, the purified polypeptide is surprisingly stable at the selected low pH.

In a further aspect, the methods of the invention includes a chromatographic step useful to isolate the polypeptide from low-molecular weight impurities (peptides having a molecular weight smaller than the polypeptide of interest). For example, those impurities may be removed using hydrophobic interaction chromatography.

The above described process steps and methods may employed in any combination to create an efficient and cost-effective polypeptide production process that can provide a recombinant polypeptide in high yield and purity and can also provide a polypeptide that is suitable for clinical applications. In some embodiments, the recombinant polypeptides so produced are glycopeptides and can be further processed to modify the structure of their glycan residues.

### The Methods

The present invention provides methods for the production of polypeptides and glycopeptides.

In one aspect, the invention provides a method of making a composition that includes a recombinant polypeptide, wherein the polypeptide is expressed in a host cell, such as a mammalian cell (e.g., CHO cell) or an insect cell (e.g., using a baculoviral expression system). Insect cell lines useful in the methods of the invention are described herein. In one embodiment, the composition made by the method of the invention is essentially free of endoglycanase activity. In another embodiment, the composition is essentially free of proteolytic activity in addition to being essentially free of endoglycanase activity. An exemplary method includes the following steps: (a) subjecting a mixture including the polypeptide (e.g., insect cell culture liquid after filtration) to anion exchange or mixed-mode chromatography including: (i) contacting the mixture and an anion exchange medium (e.g., Q-sepharose) or a mixed-mode chromatography medium having anion exchange capabilities (e.g., Capto Adhere); and (ii) eluting the polypeptide from the anion exchange or mixed-mode chromatography medium. In one example, the anion exchange medium is not Mustang Q or Q_{XL}. In one example, the polypeptide of interest is found in the flow-through fraction of the anion exchange or mixed-mode chromatography step. Anion exchange and mixed-mode media are known in the art. Exemplary media are described herein, below.

In one embodiment, the invention provides a method of removing endoglycanase activity from a polypeptide solution. The polypeptide may be expressed in a host cell, such as a mammalian cell (e.g., CHO cell) or an insect cell (e.g., using a baculoviral expression system). An exemplary method includes the following steps: (a) subjecting the polypeptide solution (e.g., insect cell culture liquid after filtration) to anion exchange or mixed-mode chromatography including: (i) contacting the solution with an anion exchange medium (e.g., Q-sepharose) or a mixed-mode chromatography medium having anion exchange capabilities (e.g., Capto Adhere); and (ii) eluting the polypeptide from the anion exchange or mixed-mode chromatography medium.

In one example according to any of the above embodiments, the anion exchange or mixed-mode chromatography medium is a strong anion exchanger and includes, for example, a ligand having a quaternary amino group. In another example, the anion exchange or mixed-mode ligand includes a tertiary amino group. In a particularly preferred embodiment, the anion exchanger is a mixed-mode medium incorporating a mixed-mode ligand having a quaternary amino group.

The mixed-mode medium may further provide hydrophobic interaction capabilities. For example, the mixed-mode medium includes a mixed-mode ligand having a hydrophobic moiety. Exemplary hydrophobic moieties include linear or branched unsubstituted alkyl, unsubstituted aryl, unsubstituted heteroaryl, alkyl-substituted aryl or alkyl-substituted heteroaryl groups. Each of these groups may optionally include at least one heteroatom (e.g., e.g., an amide bond or an ether or thioether group). In yet another example, the mixed-mode ligand may provide hydrogen-bonding capabilities. For example, the ligand includes a moiety having at least one functional group that is capable of forming hydrogen bonds with either hydrogen bond donor- or hydrogen bond acceptor-groups on the polypeptide. In one example, the mixed-mode ligand includes a moiety having at least one hydroxyl group. An exemplary mixed-mode medium useful in the methods of the invention combines anion exchange capabilities with both, hydrophobic interaction capabilities and hydrogen-bonding capabilities. One medium having those characteristics is Capto Adhere.

In one example the mixed-mode ligand comprises the moiety:

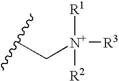

wherein R¹ is C₁-C₁₀ alkyl (e.g., methyl, ethyl, propyl, butyl); R² is a hydrophobic moiety described herein below and R³ is a moiety including at least one hydroxyl group (e.g., CH₂CH₂OH).

In another example the mixed-mode ligand comprises the moiety:

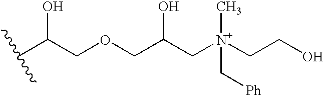

The above described method may further include: (b) subjecting a mixture that includes the polypetide, to dye-ligand affinity chromatography. In one example, the mixture subjected to dye-ligand affinity chromatography is the flow-though fraction from the anion exchange or mixed-mode step, described above. In one example according to this embodiment, the dye-ligand affinity chromatography includes the following steps: (iii) contacting the flow-through fraction and a dye-ligand affinity chromatography medium; and (iv) eluting the polypeptide from the dye-ligand affinity chromatography medium generating an eluate fraction containing the polypeptide. In one example, the polypeptide is reversibly bound (retained) by the dye-ligand affinity chromatography medium under the conditions used to apply the polypeptide (polypeptide capture). The column may be washed using a wash buffer that does not elute the polypeptide. An elution buffer may then be used to elute the reversibly bound polypeptide from the dye-ligand affinity chromatography medium. In one example the elution buffer includes a high salt content (e.g., 2 M KCl) and may optionally include an amino acid, such as glycine or arginine. Dye-ligand affinity chromatography media are known in the art. Exemplary media are disclosed herein, below. The dye-ligand affinity medium can optionally be replaced with a cation exchange medium.

In one example according to any of the above embodiments, the dye-ligand affinity chromatography medium includes Cibacron Blue or an analog thereof immobilized on a solid support. Cibacron Blue resins are art recognized (see e.g., Subramanian S, CRC Critical Reviews in Biochemistry 1984, 16(2): 169-205, which is incorporated herein by reference in its entirety) and are distinguished by the chemical structure of the dye molecule and the linker used to covalently link the dye-molecule to the solid support. Exemplary solid supports for Cibacron Blue resins include sepharose- and agarose-based matrices. An exemplary dye-ligand affinity medium useful in the methods of the invention is Capto Blue. In one example, the dye-ligand affinity medium has a binding capacity for human serum albumin (HSA) that is at least about 20 mg HSA/mL and preferably at least about 25 mg HSA/mL. In another example, the Cibacron Blue resin can bind about 30 mg HSA/mL resin.

In one example according to any of the above embodiments, the method of the invention includes anion exchange or mixed-mode chromatography in combination with dye-ligand affinity chromatography. For example, mixed-mode chromatography is performed prior to dye-ligand affinity chromatography. In another example, the flow-through fraction from the mixed-mode chromatography step is contacted with a dye-ligand affinity medium essentially immediately after it elutes from the mixed-mode medium, e.g., within 2 hours of elution and preferably within 1 hour of elution. In a particular example, the mixed-mode and dye-ligand affinity steps are arranged in a continuous-flow processing module by connecting the two media so that the flow-through from the mixed-mode filtration step is not collected but enters the dye-ligand affinity column directly upon elution. An exemplary arrangement of processing steps according to this embodiment is illustrated in FIG. 2.

In one embodiment, anion exchange or mixed-mode chromatography is useful to isolate the desired polypeptide from unwanted proteins, such enzymes derived from the insect-cell expression system. The inventors have discovered that certain anion exchange or mixed-mode resins are particularly useful to remove endo-glycanases (endo-glycosidases), which are enzymes that can cleave glycosyl moieties from existing glycan residues attached to the polypeptide. These reactions are highly undesired because they can reduce or destroy the biological activity of the polypeptide and/or compromise the homogeneity of the polypeptide population effecting product quality.

Hence, in one example, the anion exchange or mixed-mode chromatography step of any of the above embodiments, is useful to reduce endoglycanase activity of the polypeptide solution. For example, endoglycanase activity is measured before and after the polypeptide solution is processed using anion exchange or mixed-mode chromatography, optionally followed by dye-ligand affinity or cation exchange chromatography. In one example, the polypeptide solution after anion exchange or mixed-mode chromatography is essentially free of endoglycanase activity.

In a particular example, the polypeptide is processed using mixed-mode chromatography followed by dye-ligand affinity chromatography and the eluate fraction from the dye-ligand affinity step is essentially free of endoglycanase activity. For example, the polypeptide solution after mixed-mode and dye-ligand affinity chromatography has an endoglycanase activity that is less than about 5%, less than about 4%, less than about 3%, less than about 2% or less than about 1% of the endoglycanase activity found in the polypetide solution prior to mixed-mode chromatography and dye-ligand affinity chromatography. In another example, the residual endoglycanase activity after anion exchange or mixed-mode chromatography is preferably less than about 0.5% and more preferably less than about 0.4%, less than about 0.3% or less than about 0.1%.

Unexpectedly, the inventors have also determined that the presence of calcium ions (e.g., due to addition of CaCl₂) in the polypeptide solution (e.g., after filtration to remove cellular debris and before anion exchange or mixed-mode chromatography) significantly enhances endoglycanase activity. For example, the endoglycanase activity in a hollow fiber filtered polypeptide solution containing 10 mM CaCl₂ is enhanced by about 80% compared to a control sample not supplemented with a calcium salt. Hence, in one example, in order to minimize degradation of the polypeptide by endoglycanases, addition of calcium ions to the polypeptide solution is avoided. It is especially useful not to add calcium ions to the culture liquid before the sample is eluted from an anion exchange or mixed-mode resin because such material may still contain comparably high concentrations of harmful endoglycanases. Hence, in one example according to any of the above described embodiments, the feed for the anion exchange or mixed-mode chromatography step is not supplemented with calcium ions. For example, the Ca²⁺ concentration in the anion exchange or mixed-mode feed and/or loading buffer is less than about 10 mM, preferably less than about 5 mM, and more preferably less than about 1 mM. It was also noted that cold temperatures (e.g., 4 C) significantly reduce endoglycanase activity. Hence, in one embodiment, the polypeptide solution before elution from an anion exchange or mixed-mode medium is kept at a temperature below about 10° C., preferably below about 5° C. and most preferably at about 4° C.

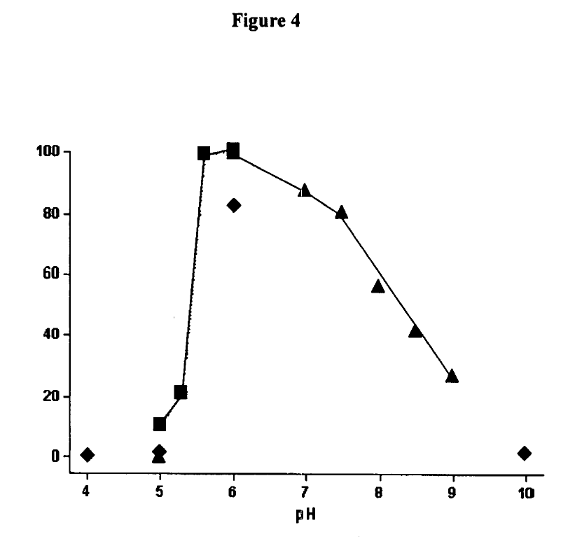

It was further discovered that the endoglycanase activity is largely dependent on the pH of the polypeptide solution. In one example, the pH maximum for the endoglycanase(s) is about pH 6 and rapid loss of endoglycanase activity is observed when lowering the pH below 6.0. This pH dependency is illustrated in FIG. 4. Hence, in one example according to any of the above embodiments, the pH of the polypeptide solution before elution from an anion exchange or mixed-mode medium is kept at or adjusted to below about pH 6, preferably at about pH 5.9, more preferably at about pH 5.8 and most preferably at about pH 5.7. In another example, the pH is below about 5.7, below about 5.6, below about 5.5, below about 5.4, below about 5.3, below about 5.2, below about 5.1, below about 5.0. For example, the pH of the culture medium is adjusted to a pH below 6.0 either before or after filtration to remove cellular debris.

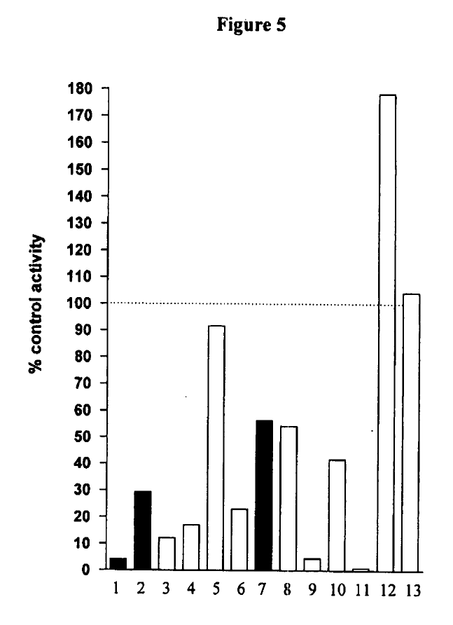

In addition, a series of additives were examined for their effect on endoglycanase activity. Results are summarized in FIG. 5. For example, it was discovered that addition of an inhibitor, such as ZnCl₂, KCl or a guanidine salt (e.g., to the culture liquid before or after filtration to remove cellular debris) can be beneficial to the reduction of enzymatic degradation of the polypeptide by endoglycanases.

The inventors have further discovered that subjecting the polypeptide solution to at least one freeze-thaw cycle lowers the endoglycanase activity present in the polypeptide solution. In one example, cooling the polypeptide solution to a temperature below 0° C., reduces the endoglycanase activity to less than about 50%. In another example, the polypeptide solution is cooled to below about −5° C., below about −10° C., below about −15° C. or below about −20° C. to reduce the endoglycanase activity to less than about 40% (e.g., less than about 30%, less than about 20%, less than about 10% or less than about 5%) of the original activity before freezing.

In one embodiment, the polypeptide solution is kept frozen at any of the above listed temperatures for less than about 48 hours, less than about 24 hours, less than about 20 hours, less than about 18 hours, less than about 16 hours, less than about 14 hours, less than about 12 hours, less than about 10 hours, less than about 8 hours, less than about 6 hours, less than about 4 hours, less than about 2 hours or less than about 1 hour to reduce the endoglycanase activity to 50% or less.

The inventors have further discovered that the polypeptide can be isolated from certain proteases using anion exchange or mixed-mode chromatography, in combination with dye-ligand affinity or cation exchange chromatography. In one example, the anion exchange medium is not Mustang Q or Q_{XL}. In another example, the polypeptide is processed using mixed-mode chromatography, wherein the mixed-mode medium comprises anion exchange capabilities, followed by dye-ligand affinity chromatography or cation exchange chromatography and the eluate fraction from the dye-ligand affinity or cation exchange chromatography step is essentially free of proteolytic activity. For example, the polypeptide solution after mixed-mode and dye-ligand affinity chromatography has a proteolytic activity that is less than about 10%, less than about 5%, less than about 4% or less than about 3% of the proteolytic activity of the polypeptide solution prior to mixed-mode chromatography and dye-ligand affinity chromatography. In one example the residual proteolytic activity of the polypeptide solution after mixed-mode and dye-ligand affinity chromatography is less than about 2%, less than about 1.8%, less than about 1.6% or less than about 1.4%.

The inventors have further discovered that the combination of anion exchange or mixed-mode chromatography followed by cation exchange or dye-ligand affinity chromatography, represents a fast and efficient method to enrich the polypeptide to a certain purity. In this embodiment the polypeptide is found in the flow-through fraction of the anion exchange or mixed-mode step and is consequently captured by the cation exchange or dye-ligand affinity medium. It is then eluted from the cation exchange or dye-ligand affinity medium using an appropriate elution buffer, such as 2M KCl. This combination is especially efficient when the two purification steps are linked into a continuous flow process module. In one example, the polypeptide solution after mixed-mode chromatography and dye-ligand affinity chromatography has a purity of at least about 20%, at least about 22%, at least about 24%, at least about 26% or at least about 28% (w/w). In another example, the polypeptide solution after mixed-mode chromatography and dye-ligand affinity chromatography has a purity of at least about 30%, at least about 32%, at least about 34%, at least about 36%, at least about 38%, at least about 40% or more than 40% (w/w).

Because the method of the invention useful to remove endoglycanases and proteases early in the purification process, it makes it possible to isolate the polypeptide in the absence of enzyme inhibitors, such as protease and endoglycanase inhibitors. Hence, in one example, the polypeptide is isolated in the absence of a protease inhibitor.

It was also discovered that anion exchange or mixed-mode chromatography followed by cation exchange or dye-ligand affinity chromatography as described in any of the above embodiments, results in unexpectedly high overall recovery (yield) of polypeptide over these two steps. For example, at least 50%, at least 55%, at least 60% or at least 65% of the polypeptide that is loaded onto the mixed-mode medium is recovered in the eluate fraction of the dye-ligand affinity chromatography step. In another example, at least 70%, at least 71%, at least 72%, at least 73%, at least 74%, at least 75%, at least 76%, at least 77%, at least 78%, at least 79% or at least 80% of the polypeptide are recovered after processing the polypeptide solution by mixed-mode and dye-ligand affinity chromatography.

In one exemplary embodiment, the method of the invention combines anion exchange or mixed-mode chromatography and cation exchange or dye-ligand affinity chromatography with a processing step that is useful for the inactivation of viruses that may be contained in the polypeptide solution. In one example, inactivation of viruses is accomplished using UV irradiation (e.g., using UVC light) of the polypeptide solution in a manner that minimizes harm to the desired polypeptide. In another example, viral inactivation is accomplished using a low-pH hold procedure described herein and in U.S. patent application Ser. No. 11/396,215 filed Mar. 30, 2006, the disclosure of which is incorporated herein in its entirety. It was discovered that certain polypeptides can withstand surprisingly low pH conditions, while most viruses do not survive those conditions.

Hence, in another aspect, the invention provides a method of making a composition including a recombinant polypeptide of the invention, wherein the composition is essentially free of endoglycanase activity and essentially free of proteolytic activity. The method includes: (a) eluting a mixture including the polypeptide from an anion exchange or mixed-mode chromatography medium comprising a mixed-mode ligand providing anion exchange capabilities (e.g., having a quaternary amino group). In one example, the desired polypeptide is found in the flow-through fraction of this anion exchange step. In another example, the polypeptide is bound to the anion exchange medium and is eluted with an elution buffer. The method further includes: (b) contacting a mixture containing the polypeptide (e.g. the flow-through fraction from the anion exchange or mixed-mode step containing the polypeptide) with a cation exchange or dye-ligand affinity chromatography medium; (c) eluting the polypeptide from the cation exchange or dye-ligand affinity chromatography medium thereby producing an eluate fraction including the polypeptide.

The method further includes: irradiating a mixture including the polypeptide (e.g., the eluate fraction of step (c)) with UV light in a manner sufficient to effect viral inactivation. Alternatively, the mixture including the polypeptide (e.g., the eluate fraction of step (c)) is subjected to a low pH hold procedure. In an exemplary embodiment, the low pH hold procedure includes the following steps: (i) lowering the pH of the mixture (e.g., the eluate fraction of step (c)) to a first pH value (e.g., between about 2.5 and about 4.0); (ii) maintaining the first pH value for a selected period of time (e.g., between about 30 min and about 2 hours); and (iii) raising the pH of the eluate fraction (e.g., to about 6.0).

In one example, UV irradiation is performed after cation exchange or dye-ligand affinity chromatography. In another example, the low pH hold step is performed after the cation exchange or dye-ligand affinity step. In another example, the polypeptide is processed by mixed-mode chromatography, followed by dye-ligand affinity chromatography, followed by low-pH hold or UV irradiation.

In yet another example, the flow-through fraction from the mixed-mode chromatography step is contacted with a dye-ligand affinity medium essentially immediately after it elutes from the mixed-mode medium as described herein above. In a particular example, the mixed-mode and dye-ligand affinity steps are arranged in a continuous-flow processing module by connecting the two media so that the flow-through from the mixed-mode filtration step is not collected but enters the dye-ligand affinity column directly upon elution. An exemplary arrangement of processing steps according to this embodiment is illustrated in FIG. 2.

In another example according to any of the above described embodiments, the method of the invention further includes at least one membrane filtration step, wherein the polypeptide solution is passed through a membrane that has a molecular weight cutoff (MWCO) sufficient to remove viral particles from the polypeptide solution. Such virus filters are known in the art. In one example, the virus filter includes a polyethersulfone membrane. Exemplary filters include Viresolve NFP and Planova (e.g., Planova 20N) filters.

In another example according to any of the above described embodiments, the method of the invention may further include (in addition to the described anion exchange or mixed-mode chromatography and cation exchange or dye-ligand affinity steps): eluting the polypeptide from at least one, preferably at least two different chromatography media. Each additional chromatography medium is selected from a hydrophobic interaction chromatography (HIC) medium, a cation exchange chromatography medium, an anion exchange chromatography medium and a hydroxyapatite or fluoroapatite chromatography medium. In one embodiment, the polypeptide is eluted from a mixed-mode filter and a dye-ligand affinity resin before it is subjected to HIC and cation exchange chromatography (e.g., using a sulphopropyl resin). Ion exchange chromatography, HIC, hydroxyapatite and fluoroapatite chromatography are known in the art. Exemplary procedures useful in the methods of the invention are described herein, below. In a preferred embodiment, the polypeptide purification process of the invention does not include reverse-phase chromatography. If hydrophobic chromatography is needed, HIC is preferably used.

An exemplary method according to any of the above embodiments, further includes: infecting insect cells (e.g., Spodoptera frugiperda cells) in an insect cell culture with a recombinant baculovirus comprising a nucleotide sequence encoding the polypeptide. In one embodiment, the insect cells are infected with the baculovirus in a cell culture medium that is supplemented with a lipid, for example, a lipid mixture disclosed herein, below and in U.S. patent application Ser. No. 11/396,215 filed Mar. 30, 2006, the disclosure of which is incorporated herein in its entirety.

In one example, the lipid mixture includes an alcohol (e.g. ethanol), a sterol (e.g. cholesterol), a surfactant (e.g. block copolymer Pluronic F68), a non-ionic detergent (e.g. Tween-80), an antioxidant (e.g. tocopherols, such as alpha- or delta-tocopherol acetate), and a lipid source. Exemplary lipid sources include oils, such as fish oils (e.g., cod liver oil), oil or fat components, such as fatty acids or their derivatives (e.g., C₁-C₆ alkyl esters). In one example, the lipid source includes fatty acids from fish oil, such as cod liver oil and/or methyl esters of those fatty acids. An exemplary lipid mix composition is disclosed in Table 1, below.

TABLE 1
Exemplary Lipid Mixture Components

| COMPONENT | AMOUNT/1 L |
|---|---|
| Ethanol | 100.00 mL |
| Cholesterol | 450.00 mg |
| Tween 80 | 2500.00 mg |
| Cod Liver Oil | 1700.00 mg |
| (+)-α-Tocopherol Acetate | 300.00 mg |
| Pluronic F-68 (10%) | 900.00 mL |

In one example, the lipid mixture of the invention is supplemented into the insect cell culture at a percentage of total culture volume equivalent to between about 0.5% and about 3% v/v (e.g., 1.5%). In another example, the lipid mixture is added to supplement the insect cell culture from between about 0.5 hours to about 2.0 hours (e.g., hour) prior to infecting the culture with an expression vector (e.g., baculovirus). In another example, the lipid mixture is prepared just prior (e.g., less than about 5 hours, less than about 4 hours, less than about 3 hours, less than about 2 hours or less than about 1 hour prior to adding the lipid mixture to the fermentation culture.

In one example according to any of the above embodiments, the method of the invention further includes: expressing the polypeptide in insect cells thereby forming a culture liquid comprising the polypeptide. The method may further include: removing cellular debris from the culture liquid. In one example, cellular debris and other particles are are removed from the culture liquid using filtration, such as hollow fiber filtration or depth filtration. In another example, hollow fiber filtration is combined with anion exchange or mixed-mode chromatography and cation exchange or dye-ligand affinity chromatography in a single-unit operation, for example as outlined in FIG. 2. In one example, combining these processing steps in a single-unit operation significantly reduces processing times. In an exemplary embodiment, the time required to perform hollow fiber filtration, mixed-mode chromatography and dye-ligand affinity chromatography on a large-scale (e.g., 15-1500 liter) is less than about 5 hours, less than about 4 hours, less than about 3 hours, less than about 2 hours or less than about 1.5 hours.

In one example, the polypeptide in any of the above discussed methods is ST6GalNAc1. In another example, the polypeptide in any of the above discussed methods is erythropoietin (EPO). In yet another example, the polypeptide in any of the above discussed methods includes a substantially uniform, insect-specific glycosylation pattern.

Thus, in another aspect, the invention provides a method of making a composition including a recombinant EPO polypeptide, wherein the EPO polypeptide is expressed in an insect cell (e.g., Sf9) and the composition is essentially free of endoglycanase activity and optionally essentially free of proteolytic activity. The method includes: (a) subjecting a mixture including the EPO polypeptide to anion exchange or mixed-mode chromatography (e.g., mixed-mode filtration), wherein the mixed-mode medium has anion exchange capabilities (e.g., mixed-mode ligand includes quaternary amino group) and at least one additional capability selected from hydrophobic interaction capability (e.g., the mixed-mode ligand includes a hydrophobic moiey described herein) and hydrogen-bonding capability (e.g., the mixed-mode ligand includes a moiety having at least one hydroxyl group). The anion exchange or mixed-mode procedure may include the following steps: (i) contacting the mixture and an anion exchange or mixed-mode chromatography medium; and (ii) eluting the polypeptide from the anion exchange or mixed-mode chromatography medium. In one example, the polypeptide is contained in the flow-through fraction of the anion exchange or mixed-mode step.

In yet another aspect, the invention provides a composition made by any of the above described methods.

### I. Polypeptides

The polypeptide produced by methods of the present invention can be any recombinant polypeptide expressed in a host cell. The polypeptide can be a glycopeptide and can have any number of amino acids. In one embodiment, the polypeptide of the invention has a molecular weight of about 5 kDa to about 500 kDa. In another embodiment, the polypeptide has a molecular weight of about 10 kDa to about 100 kDa. In yet another embodiment, the polypeptide has a molecular weight of about 10 kDa to about 30 kDa. In a further embodiment, the polypeptide has a molecular weight of about 20 kDa to about 25 kDa.

Exemplary polypeptides include wild-type polypeptides and fragments thereof as well as polypeptides, which are modified from their naturally occurring counterpart (e.g., by mutation or truncation). A polypeptide may also be a fusion protein. Exemplary fusion proteins include those, in which the polypeptide is fused to a fluorescent protein (e.g., GFP), a therapeutic polypeptide, an antibody, a receptor ligand, a proteinaceous toxin, MBP, a His-tag, and the like.

In one embodiment, the polypeptide is a therapeutic polypeptide, such as those currently used as pharmaceutical agents (i.e., authorized drugs). A non-limiting selection of polypeptides is shown in FIG. 28 of U.S. patent application Ser. No. 10/552,896 filed Jun. 8, 2006, which is incorporated herein by reference.

Exemplary polypeptides include growth factors, such as hepatocyte growth factor (HGF), nerve growth factors (NGF), epidermal growth factors (EGF), fibroblast growth factors (e.g., FGF-1, FGF-2, FGF-3, FGF-4, FGF-5, FGF-6, FGF-7, FGF-8, FGF-9, FGF-10, FGF-11, FGF-12, FGF-13, FGF-14, FGF-15, FGF-16, FGF-17, FGF-18, FGF-19, FGF-20, FGF-21, FGF-22 and FGF-23), blood coagulation factors (e.g., Factor V, Factor VII, Factor VIII, B-domain deleted Factor VIII, partial B-domain deleted Factor VIII, vWF-Factor VIII fusion (e.g., with full-length, B-domain deleted Factor VIII or partial B-domain deleted Factor VIII), Factor IX, Factor X and Factor XIII), hormones, such as human growth hormone (hGH) and follicle stimulating hormone (FSH), as well as cytokines, such as interleukins (e.g., IL-1, IL-2, IL-3, IL-4, IL-5, IL-6, IL-7, IL-8, IL-9, IL-10, IL-11, IL-12, IL-13, IL-14, IL-15, IL-16, IL-17, IL-18) and interferons (e.g., INF-alpha, INF-heta, INF-gamma).

Other exemplary polypeptides include enzymes, such as glucocerebrosidase, alpha-galactosidase (e.g., Fabrazyme™), acid-alpha-glucosidase (acid maltase), iduronidases, such as alpha-L-iduronidase (e.g., Aldurazyme™), thyroid peroxidase (TPO), beta-glucosidase (see e.g., enzymes described in U.S. patent application Ser. No. 10/411,044), arylsulfatase, asparaginase, alpha-glucoceramidase, sphingomyelinase, butyrylcholinesterase, urokinase and alpha-galactosidase A (see e.g., enzymes described in U.S. Pat. No. 7,125,843).

Other exemplary parent polypeptides include bone morphogenetic proteins (e.g., BMP-1, BMP-2, BMP-3, BMP-4, BMP-5, BMP-6, BMP-7, BMP-8, BMP-9, BMP-10, BMP-11, BMP-12, BMP-13, BMP-14, BMP-15), neurotrophins (e.g., NT-3, NT-4, NT-5), erythropoietins (EPO), growth differentiation factors (e.g., GDF-5), glial cell line-derived neurotrophic factor (GDNF), brain derived neurotrophic factor (BDNF), nerve growth factor (NGF), von Willebrand factor (vWF), vWF-cleaving protease (vWF-protease, vWF-degrading protease), granulocyte colony stimulating factor (G-CSF), granulocyte-macrophage colony stimulating factor (GM-CSF), α₁-antitrypsin (ATT, or α-1 protease inhibitor), tissue-type plasminogen activator (TPA), hirudin, leptin, urokinase, human DNase, insulin, hepatitis B surface protein (HbsAg), human chorionic gonadotropin (hCG), chimeric diphtheria toxin-IL-2, glucagon-like peptides (e.g., GLP-1 and GLP-2), anti-thrombin III (AT-III), prokinetisin, CD4, α-CD20, tumor necrosis factor receptor (TNF-R), P-selectin glycoprotein ligand-1 (PSGL-1), complement, transferrin, glycosylation-dependent cell adhesion molecule (GSyCAM), neural-cell adhesion molecule (N-CAM), TNF receptor-IgG Fc region fusion protein, extendin-4, BDNF, beta-2-microglobulin, ciliary neurotrophic factor (CNTF), fibrinogen, GDF (e.g., GDF-1, GDF-2, GDF-3, GDF-4, GDF-5, GDF-6-15), SDNF and GLP-1. Exemplary amino acid sequences for some of the above listed polypeptides are described in U.S. Pat. No. 7,214,660, all of which are incorporated herein by reference.

In an exemplary embodiment, the polypeptide is EPO comprising the amino acid sequence of (SEQ ID NO: 1), which is shown below:

Ala Pro Pro Arg Leu Ile Cys Asp Ser Arg Val Leu Glu Arg Tyr Leu Leu Glu Ala Lys Glu Ala Glu Asn²⁴ Ile Thr Thr Gly Cys Ala Glu His Cys Ser Leu Asn Glu Asn³⁸ Ile Thr Val Pro Asp Thr Lys Val Asn Phe Tyr Ala Trp Lys Arg Met Glu Val Sly Gln Gln Ala Val Glu Val Trp Gln Gly Leu Ala Leu Leu Ser Glu Ala Val Leu Arg Gly Gln Ala Leu Leu Val Asn⁸³ Ser Ser Gln Pro Trp Glu Pro Leu Gln Leu His Val Asp Lys Ala Val Ser Gly Leu Arg Ser Leu Thr Thr Leu Leu Arg Ala Leu Gly Ala Gln Lys Glu Ala Ile Ser Pro Pro Asp Ala Ala Ser¹²⁶ Ala Ala Pro Leu Arg Thr Ile Thr Ala Asp Thr Phe Arg Lys Leu Phe Arg Val Tyr Ser Asn Phe Leu Arg Gly Lys Leu Lys Leu Tyr Thr Sly Glu Ala Cys Arg Thr Gly Asp

In an exemplary embodiment, the EPO polypeptide includes an amino acid sequence according to SEQ ID NO: 1 having at least one mutation replacing a basic amino acid residue, such as arginine or lysine, with an uncharged amino acid, such as glycine or alanine. In another embodiment, the EPO polypeptide includes an amino acid sequence according to SEQ ID NO: 1 having at least one mutation, selected from Arg¹³⁹ to Ala¹³⁹, Arg¹⁴³ to Ala¹⁴³ and Lys¹⁵⁴ to Ala⁵⁴.

Also within the scope of the invention are polypeptides that are antibodies. The term antibody is meant to include antibody fragments (e.g., Fe domains), single chain antibodies, Lama antibodies, nano-bodies and the like. Also included in the term are antibody-fusion proteins, such as Ig chimeras. Preferred antibodies include humanized, monoclonal antibodies or fragments thereof. All known isotypes of such antibodies are within the scope of the invention. Exemplary antibodies include those to growth factors, such as endothelial growth factor (EGF), vascular endothelial growth factors (e.g., monoclonal antibody to VEGF-A, such as ranibizumab (Lucentis™)) and fibroblast growth factors, such as FGF-7, FGF-21 and FGF-23) and antibodies to their respective receptors. Other exemplary antibodies include anti-TNF-alpha monoclonal antibodies (see e.g., U.S. patent application Ser. No. 10/411,043), TNF receptor-IgG Fc region fusion protein (e.g., Enbrel™), anti-HER2 monoclonal antibodies (e.g., Herceptin™), monoclonal antibodies to protein F of respiratory syncytial virus (e.g., Synagis™), monoclonal antibodies to TNF-α (e.g., Remicade™), monoclonal antibodies to glycoproteins, such as IIb/IIIa (e.g., Reopro™), monoclonal antibodies to CD20 (e.g., Rituxanm), CD4 and alpha-CD3, monoclonal antibodies to PSGL-1 and CEA. Any modified (e.g., mutated) version of any of the above listed polypeptides is also within the scope of the invention.

The method can optionally be used to produce enzymes (e.g., enzymes useful for the in vitro modification of glycopeptides), such as GNT1, GalT1, ST3Gal3, CST2, Sialidase, GalNAcT2, CorelGaIT, ST6GalNAc1, ST6Gal1, ST3Gal1, ST3Gal2, GalNAcT1, GalNAcT2, GalNAcT3, GalNAcT4, GalNAcT5, GalNAcT6, GalNAcT7, GalNAcT8, GalNAcT9, GalNAcT10 and GalNAcT11. In an exemplary embodiment, the polypeptide includes a substantially uniform, insect-specific glycosylation pattern.

### II. Cell Culture

### II. a) Cells

The polypeptides of the current invention can be expressed in any useful cell-line, including bacterial, mammalian and insect cell lines. In a preferred embodiment, the polypeptide is expressed in insect cells. Insect cells suitable for use in the present invention are from any order of the class Insecta. In a preferred embodiment, the insect cell can be hosts to recombinant viruses (e.g. baculovirus) or wild-type viruses, and can grow and produce recombinant polypeptides upon infection with the virus. In an exemplary embodiment, the cells are from the Diptera or Lepidoptera orders. Preferred are insect cell lines that can be used to produce polypeptides having a substantially uniform, insect-specific glycosylation pattern. In one embodiment, the polypeptide is expressed by a stably transfected cell.

About 300 insect species have been reported to have nuclear polyhedrosis virus (NPV) diseases, the majority (243) of which were isolated from Lepidoptera(see e.g., Weiss et al., Cell Culture Methods for Large-Scale Propagation of Baculoviruses, Granados et al. (eds.), The Biology of Baculoviruses: Vol. II Practical Application for Insect Control, pp. 63-87 at p. 64 (1986)). Insect cell lines derived from the following insects are exemplary: Carpocapsa pomonella (e.g., cell line CP-128); Trichoplusia ni (e.g., cell line TN-368); Autographa californica; Spodoptera frugiperda (e.g., cell line Sf9); Lymantria dispar; Mamestra brassicae; Aedes albopictus; Orgyia pseudotsugata; Neodiprion sertifer; Aedes aegypti; Antheraea eucalypti; Gnorimoschema opercullela; Galleria mellonella; Spodoptera littoralis; Drosophila melanogaster, Heliothis zea; Spodoptera exigua; Rachiplusia ou; Plodia interpunctella; Amsacta moorei; Agrotis c-nitrum; Adoxophyes orana; Agrotis segetum; Bombyx mori; Hyponomeuta malinellus; Colias eurytheme; Anticarsia gemmetalis; Apanteles melanoscelus; Arctia caja; and Lymantria dispar.

In an exemplary embodiment, the insect cells are from Spodoptera frugiperda, and in another exemplary embodiment, the cell line is a member selected from Sf9 (ATCC CRL 1711), Sf21 and High-Five insect cells. These are commonly used for baculovirus expression. Sf9 and Sf21 are ovarian cell lines from Spodoptera frugiperda. High-Five cells are egg cells from Trichoplusia ni. Sf9, Sf21 and High-Five cell lines may be grown at room temperature (e.g. 25 to 27° C.), and do not require CO₂ incubators. Their doubling time is between about 18 and 24 hours.

### II. b) Viruses

The insect cell lines cultured to produce the polypeptides and glycopeptides of the invention are preferably those suitable for the reproduction of numerous insect-pathogenic viruses such as picornaviruses, parvoviruses, entomopox viruses, baculoviruses and rhabdoviruses. In an exemplary embodiment, the baculovirus is selected from nucleopolyhedrosis viruses (NPV) and granulosis viruses (GV).

Baculoviruses are characterized by rod-shaped virus particles which are generally occluded in occlusion bodies (also called polyhedra). The family Baculoviridae can be divided in two subfamilies: the Eubaculovirinae comprising two genera of occluded viruses; nuclear polyhedrosis virus (NPV) and granulosis virus (GV), and the subfamily Nudobaculovirinac comprising the nonoccluded viruses.

Methods of preparing and using virus expression systems are generally known in the art. For example, with respect to baculovirus systems, representative references include U.S. Pat. No. 5,194,376, U.S. Pat. No. 5,147,788, U.S. Pat. No. 4,879,236 and Bedard C. et al (1994) Cytotechnology 15:129-138; Hink W T et al, (1991) Biotechnology Progress 7:9-14; Licari P. et al., (1992) Biotechnology and Bioengineering 39:614-618, each of which is incorporated herein by reference in its entirety.

Thus in one embodiment, the invention utilizes a baculovirus vector containing a nucleic acid encoding a polypeptide of the invention. The incorporation of a desired nucleic acid into a baculovirus expression vector may be accomplished using techniques that are well known in the art. For example, such techniques are described in, Sambrook et al. (Third Edition, 2001, Molecular Cloning: A Laboratory Manual, Cold Spring Harbor Laboratory, New York), and in Ausubel et at (1997), Current Protocols in Molecular Biology, John Wiley & Sons, New York).

### II. c) Composition of the Culture Media

Media for culturing insect cells are commercially available. In an exemplary embodiment Sf-900 II, available from Invitrogen, is used to grow insect cell cultures for infection with baculovirus. Sf-900 II medium is optimized to support Sf9 and Sf21 cell growth in both monolayer and suspension applications so that the cells can be used for inter alia Baculovirus Expression Vector System (BEVS) technology.

Protocols for the preparation of insect cell culture media are also known in the art (see e.g., Weiss et al., Cell Culture Methods for Large-Scale Propagation of Baculoviruses, in Granados et al (eds.), The Biology of Baculoviruses: Vol II Practical Applicationfor Insect Control, pp. 63-87 (1986)).

In general, insect cell culture media contain inorganic salts e.g., CaCl₂, MgCl₂; sugars e.g., sucrose, maltose; amino acids e.g., L-proline, L-tyrosine; and vitamins e.g., niacin and folic acid. Specific quantities of the various media components are disclosed in Schlaeger, E. (1996) Cytotechnology 20:57-70. This basic media is optionally supplemented with serum e.g., fetal bovine serum, or alternatively, with various lipid compositions.

### Lipid Mixture

Lipids are essential for the growth of insect cell cultures in serum free media. The general development of insect cell culture media is reviewed in Schlaeger, E. (1996) Cytotechnology 20: 57-70, which is incorporated herein by reference. Typically, insect cells require a culture medium comprising sterols, fatty acids, amino acids and salts for robust growth.

The present inventors have discovered that, contrary to the teachings of the prior art, the infection of insect cells with recombinant baculovirus encoding a peptide of interest in the presence of a lipid mixture, results in improved yields of the peptide when compared to yields that can be achieved if no lipids are present at the time of infection. Furthermore, in an exemplary embodiment, the quality of the peptide is improved in that the peptides so produced include a substantially uniform glycosylation pattern. The method is particularly useful for the large-scale production of glycopeptides.

In one aspect the present invention provides a lipid mixture that includes an alcohol (e.g. ethanol), a sterol (e.g. cholesterol), a surfactant (e.g. block copolymer Pluronic F68), a non-ionic detergent (e.g. Tween-80), an antioxidant (e.g. tocopherols, such as alpha- or delta-tocopherol acetate), and a lipid source (e.g. cod liver oil, cod liver oil fatty acids or methyl esters thereof).

In one embodiment according to this aspect, the lipid mixture includes an alcohol e.g., ethanol in an amount between about 5% v/v to about 20% v/v (e.g., 10% v/v), a sterol (e.g. cholesterol) in an amount between about 0.02% to about 0.06% w/v (e.g., 0.045%), a non-ionic surfactant (e.g. Pluronic F-68) in an amount between about 5% w/v to about 15% w/v, a non-ionic detergent (e.g., Tween-80) in an amount between about 0.1% w/v to about 0.3% w/v (e.g., 0.25%), an antioxidant (e.g., alpha-tocopherol acetate) in an amount between about 0.01% w/v to about 0.05% w/v (e.g., 0.03%), and a lipid source (e.g. cod liver oil fatty acid methyl esters) in an amount between about 0.05% w/v to about 0.25% w/v (e.g., 0.17%).

In another embodiment the volume of lipid mixture added to supplement the insect cell culture is a volume that is equivalent to between about 0.5% to about 3% v/v. In another embodiment, the volume of lipid mixture added to supplement the insect cell culture is a volume that is equivalent to about 1.0% to about 2.0% v/v, preferably about 1.0% to about 1.5% v/v and, more preferably, about 1.5% v/v.

In another exemplary embodiment, addition of the lipid mixture to the cell culture broth increases the titer of the desired peptide by about 10% to about 100% compared with the peptide titer when the culture broth is not supplemented with lipid mixture. A titer increase of 100% means that the amount of polypeptide present in the cell culture broth has doubled. In another exemplary embodiment, addition of the lipid mixture to the cell culture broth increases the titer of the desired polypeptide by more than about 20%, more than about 30%, more than about 40%, more than about 50%, more than about 60%, more than about 70%, more than about 80%, more than about 90% or by more than about 100%.

In one embodiment, the lipid mixture is added to the insect cell culture at a time corresponding to between about 0.5 hours to about 3.0 hours prior to infecting with a vector. In another embodiment, the lipid mixture is added about 0.5 hours to about 2 hours prior to infecting and preferably about 0.5 to about 1 hour prior to infecting with a baculovirus.

In an exemplary embodiment, the lipid mixture is prepared not more than about 48 hours prior to use, and preferably not more than about 24 hours prior to use.

### II. d) Viral Infection

### Multiplicity of Infection (MOI)

The multiplicity of infection, or MOI, represents a measure of the ratio between the number of viral particles and the number of cells to be infected by the viral particles, e.g., number of plaque forming units (pfi) per cell. The efficiency of infection is influenced by the MOI as well as the concentration of viral particles and cells.

The MOI is selected to provide a desired infection efficiency. If the number of viral particles greatly exceeds the number of cells to be infected, the cells are said to be infected at a high MOI. For example, an MOI of 5, wherein there are five times as many viral particles as cells to be infected is considered to be a high MOI. If the number of viral particles is several orders of magnitude less than the number of cells, the MOI is considered to be low.

In one embodiment, the infecting employs a multiplicity of infection between about 10⁻⁸ to about 1.0. In another embodiment, the infecting employs a multiplicity of infection between about 10⁻⁷ to about 0.5. In another embodiment, the infecting employs a multiplicity of infection between about 10⁻⁶ to about 0.2. And, in still another embodiment, the infecting employs a multiplicity of infection of about 0.1 to about 0.2.

Standard multiplicities of infection for baculovirus systems range from between about 0.8 viral particles per cell to about 0.05 particles per cell. However, baculovirus may also be infected at a much lower MOI. Co-pending and co-owned Patent Application No. PCT/US06/01582, filed Jan. 17, 2006, which is incorporated herein by reference in its entirety, discloses that a very low MOI increases yields of recombinant peptide from a baculovirus infection.

In one embodiment, a low MOI is used to initiate infection of insect cells according to the method of the invention. In this embodiment, the MOI is less than or equal to 0.00001(10⁻⁵) pfU/cell. In another embodiment, the MOI is between 0.000001(10⁻⁶) to 0.00001(10⁻⁵). In still another embodiment, the MOI is between 0.0000001(10⁻⁷) to 0.000001(10⁻⁶) or between 0.0000001(10⁻⁷) to 0.00001(10⁻⁵). In yet another embodiment, the MOI is between 0.00000001(10⁻⁸) to 0.0000001(10⁻⁷), 0.00000001(10⁻⁸) to 0.000001(10⁻⁶), or 0.00000001(10⁻⁸) to 0.00001(10⁻⁵).

It is well within the ability of the skilled artisan to determine the preferred MOI or the preferred range of MOI best suited for the production of each type or class of polypeptide to be produced according to the method of the invention. Suitable titering methods that can be used to determine the number of viable virus particles in a solution, are known in the art (e.g. standard plaque assay).

### II. e) Cell Growth

Insect cell cultures can be grown to high cell densities in bioreactors. Exemplary growth protocols are known in the art, see e.g., Weiss et al. supra. In an exemplary embodiment, the infected insect cell culture is grown for between about 50 hours to about 100 hours. In another embodiment, the infected insect culture is grown for about 60 to about 70 hours.

III. Isolation of Polypeptides from Cell Culture

In a second aspect, the current invention provides methods of purifying a recombinant peptide. The protein, which can be expressed in any suitable expression system, is first removed from the cell culture and is then further purified to remove contaminants, such as viral particles and unwanted proteins, using a variety of filtration and chromatographic purification devices.

In baculovirus expression systems, proteins are typically secreted directly from the cell into the surrounding growth media. At the conclusion of a production run, viral particles, whole cells and cellular debris are removed from the culture before the isolation of the peptide from the supernatant begins. These are generally removed by differential centrifugation, continuous centrifugation, by filtration, or by a combination of these methods.

Natural cell death, which occurs during the growth of a culture that produces directly secreted proteins, results in the release of intracellular host cell proteins and produces cellular debris. These contaminants can affect the course of the peptide production run. Indeed, the sub-cellular fragments and host cell proteins released by natural cell death are difficult to remove due to their small size.

Fortunately, insect cell cultures used to prepare recombinant peptides according to exemplary methods of the invention, experience a minimum amount of natural cell death. In an exemplary embodiment, the low level of cell death improves the quality of the culture broth at the end of a production run, which in turn improves the quality of the final peptide product. Furthermore, the improved quality of the culture broth improves the efficiency and cost effectiveness of the production run.

In addition, the inventors have discovered that when using a baculovirus expression system for the production of the peptide, one or more baculoviral protease as well as one or more baculoviral endoglycanase can contribute to the degradation of the purified peptide during the purification process. Hence, the invention provides methods for the removal of such enzymes early in the purification cascade (e.g., through ion exchange chromatography) to prevent such degradation.

Exemplary steps in a purification cascade of the invention are set forth below. It is to be understood that unless the order of steps is explicitly recited, the exemplary steps are practicable in any desired order.

### III. a) Cell Culture Harvest

In order to isolate a peptide of interest from a cell culture, cellular and other debris is removed to produce a suitable feed material for subsequent purification steps. Removing debris can be accomplished using one or more centrifugation steps, one or more filtration steps (e.g., depth filtration or hollow-fiber filtration) or a combination of centrifugation and filtration steps.

In an exemplary embodiment, wherein the cell culture volume is small, such as below about 2 liters, batch centrifugation (e.g. bottle centrifugation) can be used. In an exemplary embodiment, the supernatant is further clarified by an appropriate filter or filter train. In another exemplary embodiment, wherein a large-scale production of polypeptide is desired (e.g., from about 100 L to about 10.000 L), cell removal can be accomplished using filtration (e.g., depth filtration or hollow-fiber filtration) or optionally filtration in addition to centrifugation. In those examples the removal of debris from the cell culture is preferably accomplished using continuous centrifugation followed by filtration.

### Centrifugation

The cell culture containing the peptide can be centrifuged using any suitable centrifugation method. In an exemplary embodiment, the peptide purification process of the current invention employs a centrifugation method selected from batch centrifugation, continuous centrifugation and combinations thereof. For large-scale purification processes, centrifuges, which can be operated continuously, are most useful. These allow for the continuous addition of feedstock, the continuous removal of supernatant and the discontinuous, semi-continuous or continuous removal of solids.

In an exemplary embodiment, cell debris is removed by continuous disc-stack centrifugation. Continuous multi-chamber disc-stack centrifuges are known in the art and contain a number of parallel discs providing a large clarifying surface with a small sedimentation distance. In an exemplary embodiment, the sludge is removed through a valve. Disc-stack centrifuges may be operated either semi-continuously or continuously by using a centripetal pressurizing pump within the centrifuge bowl which forces the sludge out through a valve. The capacity and radius of such devices are large and the thickness of liquid is very small, due to the large effective surface area.

In another exemplary embodiment, centrifugation is accomplished using batch centrifugation (e.g. bottle centrifugation).

CaCl₂ is optionally added to the supernatant of the first centrifugation step. The pH of the resulting mixture is then adjusted to about pH 7.5 by adding base (e.g. sodium hydroxide). In an exemplary embodiment, upon addition of base, a precipitate forms. When NaOH is used as the base, the precipitate contains Ca(OH)₂. The precipitate is separated from the liquid (e.g. by filtration or centrifugation). In an exemplary embodiment, this “CaCl₂ precipitation” improves the performance of subsequent ultrafiltration steps.

In another exemplary embodiment, a salt of an organic acid (e.g. citrate) is added to the cell culture (e.g. prior to centrifugation). In an exemplary embodiment, citrate inhibits the activity of degrading enzymes (e.g. endoglycosidases).

### III. b) Filtration

Typically, centrifugation effectively removes the bulk of large solids, whole cells, and debris from the cell culture liquid. In addition to this first clarification step, the peptide purification process optionally includes filtration steps, which can be used as a secondary clarification step to remove particulates, virus particles, and to prevent plugging of downstream processing equipment such as membrane filters and ultrafiltration devices. In another embodiment, filtration is used as a first step for the removal of cellular debris.

### Depth Filtration

In one example, the purification process of the invention includes a depth-filtration step. Depth filtration is effective in removing residual cellular debris and other small particles. Depth filters retain contaminants using two major types of interactions between filters and contaminant particles. Particles are retained due to their size, and may also be retained due to adsorption to the filter material. Molecular and/or electrical forces between the particles and the filter material attract and retain these entities within the filter.

Depth filtration devices are known in the art. In an exemplary embodiment, the filter material is composed of a thick and fibrous cellulose structure with inorganic filter aids such as diatomaceous earth (DE) particles embedded in the openings of the fibers. This construction results in a large internal surface area, which is key to particle capture and filter capacity based on the described retention mechanisms. In another exemplary embodiment a positively charged depth filter is used.

Depth filtration can be accomplished using one or more depth filters. In an exemplary embodiment, two or more depth filters are combined into one multi-layered filter. In one example two filters are used in which the second (downstream) filter is of tighter grade. In an exemplary embodiment a depth filtration step is used subsequent to initial centrifugation of the cell culture liquid.

### Hollow Fiber Filtration

In an exemplary embodiment, the purification process of the invention includes a hollow-fiber filtration step. In one example, hollow-fiber filtration is used as the primary method for the removal of cellular debris and other particles from cell culture liquids. In one embodiment hollow-fiber filtration is used to rapidly and continuously process large-scale samples. Exemplary hollow-fiber media include, polysulfone-, polyethersulfone-(PES) and polyacrylonitrile (PAN) based membranes (e.g., those offered by GE). Exemplary hollow fiber filters have a pore size of about 0.1 μm to about 1.0 μm, preferably about 0.2 μm to about 0.8 μm, and more preferably about 0.20 μm to about 0.7 μm. In a particular example, the hollow fiber membrane has a pore size of about 0.45 μm.

In one embodiment, hollow fiber filtration can be used to reduce the volume of the culture liquid (fermentate). For example, hollow fiber filtration is used to reduce the volume of the culture liquid by about 1 fold, about 2 fold, about 3 fold, about 4 fold, about 5 fold, about 6 fold, about 7 fold, about 8 fold, about 9 fold or about 10 fold. In another example the volume of the culture liquid is reduced by more than about 10 fold, for example, about 11 fold, about 12 fold, about 13 fold, about 14 fold or about 15 to about 20 fold. The hollow fiber filtrate is optionally diafiltered to further reduce its volume and/or exchange the buffer system.

### Other Membrane Filtration

In another embodiment the peptide purification process further includes one or more membrane filtration steps to remove small particles. Exemplary membrane filters have a pore size of about 0.1 μm to about 1.0 μm, preferably about 0.1 μm to about 0.3 μm, and more preferably about 0.20 μm to about 0.25 μm.

The membrane filter is optionally part of a multi-layered filter or filter train. For example, the membrane filter is combined with one or more depth filter to form a multi-layered filter device. In an exemplary embodiment the membrane filter forms the most downstream layer of the multi-layered filter device or filter train.

### Tangential Flow Filtration (TFF)

Membrane filtration is a separation technique widely used for clarifying, concentrating, and purifying peptides. Tangential flow filtration, or cross-flow filtration, is a pressure driven separation process that uses membranes to separate components in a liquid solution or suspension based on their size and charge differences. During cross-flow separation, a feed stream is introduced into the membrane element under pressure and passed across the membrane surface in a controlled flow path. A portion of the feed passes through the membrane and is called permeate. The portion of the feed that does not cross the membrane is called retentate.

In one aspect the present invention provides a method of purifying a recombinant peptide, wherein the method includes (a) conditioning a mixture containing the peptide using a tangential flow filtration cascade. According to the method, the conditioning occurs prior to subjecting the mixture to chromatographic purification steps. The method is useful for removing baculovirus and other particles from the peptide solution and then concentrating the semi-purified peptide. The conditioning is accomplished by filtering the peptide solution through a set of ultrafiltration (UF) membranes having a molecular weight cut-off (MWCO) between about 5 kDa and about 200 kDa. The TFF cascade can include any number of high and low MWCO membranes. In one exemplary embodiment, the TFF cascade includes two membrane filters, in which the membranes have a MWCO selected according to the size of the peptide being purified. The two membrane filters can have the same or different MWCO.

In one exemplary embodiment, the peptide being purified has a molecular size that is relatively small compared to the size of certain contaminants. In one embodiment, the current invention provides ultrafiltration and diafiltration strategies that are uniquely tailored to separate small peptides from larger contaminants.

In an exemplary embodiment the TFF cascade includes two membrane filters, in which one membrane filter has a MWCO larger than the purified peptide and another membrane filter has a MWCO smaller than the purified peptide.

An exemplary method contains the following steps to condition a mixture that contains the peptide: (i) ultrafiltering the peptide solution across a first ultrafiltration membrane with a MWCO larger than the purified peptide; (ii) ultrafiltering the permeate from step (i) across a second ultrafiltration membrane with a MWCO smaller than the purified peptide; and (iii) collecting the retentate from step (ii). Preferably, the purified peptide flows through the pores of the first ultrafiltration membrane and is contained in the flow-trough (permeate) of this first ultrafiltration step. Larger proteins such as certain degrading enzymes are thus removed. During the second ultrafiltration step the purified peptide does preferably not cross the membrane and is preferably found in the retentate fraction. This allows the peptide to be concentrated and the buffer system to be altered. The buffer system is altered by replenishing the retentate reservoir with the new buffer. During this “diafiltration” step the original buffer is gradually diluted with the new “diafiltration” buffer.

Ultrafiltration Using a Membrane with a Large MWCO

In an exemplary embodiment, the purification process is initiated by filtering the TFF feed across a first membrane to produce a permeate stream while avoiding the formation of a retentate stream. In an exemplary embodiment, filtration is effected using a transmembrane pressure between about 1 and about 30 psi and a UF filter membrane with a MWCO of between about 75 kDa to about 125 kDa and preferably about 100 kDa. The ultrafiltration membrane retains baculovirus particles and other large molecular contaminants, such as larger proteins, while permitting passage of the purified peptide.

In another exemplary embodiment, the membrane utilized in this ultrafiltration step is a member selected from cellulose acetate, regenerated cellulose, and polyethersulfone. Suitable membranes include those, in which the membrane polymer is chemically modified. In a preferred embodiment, the membrane is regenerated cellulose.

Ultrafiltration Using a Membrane with a Small MWCO

In an exemplary TFF cascade, the feed is passed through an ultrafiltration membrane with a MWCO suitable to concentrate the purified peptide. To concentrate a sample, the membrane is chosen to have a MWCO that is substantially lower than the molecular weight of the purified peptide. In general, the ultrafiltration membrane is selected to have a MWCO that is 3 to 6 times lower than the molecular weight of the peptide to be retained by the membrane. If the flow rate or the processing time is of major consideration, selection of a membrane with a MWCO toward the lower end of this range (e.g. 3×) will yield higher flow rates. If recovery of peptide is the primary concern, a tighter membrane (e.g. 6×) is selected (typically with a slower flow rate).

In another exemplary embodiment, filtration is effected using a transmembrane pressure between about 1 and about 30 psi and a filter membrane with a MWCO of between about 5 kDa to about 15 kDa, and preferably 10 kDa. The second filtration step produces a retentate stream and a permeate stream. The retentate is recycled to a reservoir for the peptide solution feed under conditions of essentially constant peptide concentration in the feed by adding a buffer solution to the retentate.

The surface area of the filtration membrane used will generally depend on the amount of peptide to be purified. The membrane may be made of a low-binding material to minimize adsorptive losses and is preferably durable, cleanable, and chemically compatible with the buffers to be used. A number of suitable membranes are commercially available, including, e.g., cellulose acetate, regenerated cellulose and polyethersulfone membranes. Suitable membranes include those in which the membrane polymer is chemically modified. In an exemplary embodiment the membrane is regenerated cellulose.

The flow rate will be adjusted to maintain a constant transmembrane pressure. Generally, filtration will proceed faster with higher pressures and higher flow rates, but higher flow rates may also result in damage to the peptide or loss of peptide due to passage through the membrane. In addition, various TFF devices may have certain pressure limitations on their operation, and the pressure is adjusted according to the manufacturer's specification. In an exemplary embodiment, the pressure is between about 1 to about 30 psi, and in another exemplary embodiment the pressure is between about 8 psi to about 10 psi. Typically, the circulation pump is a peristaltic pump or diaphragm pump in the feed channel and the pressure is controlled by adjusting the retentate valve.

Subsequent to a filtration step or at the conclusion of the TFF cascade, the retentate is collected. Water or an aqueous buffer (e.g. diafiltration buffer) may be used to wash the membrane filter and recover any peptide retained by the membrane. The wash liquid may be combined with the original retentate containing the concentrated peptide. The retentate is optionally dialyzed against a buffer such as TRIS or HEPES before the partially purified peptide is subjected to subsequent purification steps, such as anion exchange chromatography.

The use of cross-flow filtration (e.g. ultrafiltration and diafiltration) prior to purification of the peptide by chromatographic means, has several unexpected advantages. First, a large part of the viral particles are removed early in the purification process. Second, the overall performance of the peptide purification process is increased. Due to the removal of large-molecular weight contaminants early in the process, the performances of downstream purification steps are significantly increased. Smaller membrane areas and smaller chromatography columns are needed in subsequent purification procedures due to generally cleaner loads.

In addition, removing degrading enzymes from the peptide solution early in the process increases the stability of the peptide during the process and overall yields are thus improved. Due to increased stability of the peptide, subsequent purification steps can optionally be performed at controlled room temperature, eliminating the need to perform the entire purification process in a cold-room facility. Short-term storage of purified peptide (e.g. overnight hold) before shipment and further processing becomes possible.

### III. d) Chromatographic Purification of Recombinant Peptides

A variety of recognized chromatographic techniques, such as size exclusion chromatography (gel filtration), ion exchange chromatography, hydrophobic interaction chromatography (HIC), affinity chromatography, mixed-mode chromatography, hydroxyapatite and fluoroapatite chromatography are used for the isolation of peptides and proteins. In an exemplary embodiment, the peptide purification process of the invention employs a combination of several chromatographic techniques. The order in which these steps are performed is dependent on the nature of the polypeptide being purified and the nature of the contaminants to be removed.

Suitable techniques for the practice of the invention separate the polypeptide of interest from a variety of contaminants on the basis of charge, degree of hydrophobicity, and/or size. Different chromatographic resins and membranes are available for each of these techniques, allowing accurate tailoring of the purification scheme to the particular peptide being purified.

In one chromatographic technique, the components in a mixture interact differently with the column material and move at different rates along the column length, achieving a physical separation that increases as they pass further down the column. In another chromatographic technique, components of the mixture, including the peptide of interest, adhere selectively to the separation medium (capture), while other components are found in the flow-through. The initially retained components are then eluted differentially by varying the composition of the solvent or buffer system. In another approach, the desired components are found in the flow-through while impurities are retained on the column and thus removed from the mixture.

### Expanded Bed Adsorption (EBA) Technology

In one embodiment of the invention, EBA technology is used to isolate the polypeptide from cell culture. This separation technique can be performed at any step during the purification process. In EBA technology, the adsorbent media is expanded by an upward liquid flow to increase the distance between the chromatographic beads. Given the created distance, particulate material is allowed to pass through the column without clogging the system. The result is a simple and scaleable separation system that combines clarification, concentration and purification into one process step. A significant positive side effect of the expanded bed system compared with packed bed systems is related to the back pressure issue. As there is no particular back pressure in expanded bed systems the flow rate limitations are associated to adsorbent density and size. Typically, flow rates in expanded bed systems are 10 times faster than in packed bed systems.

In addition, purification steps that are carried out early in the purification cascae and in which the polypeptide is captured from the cell culture liquid, are typically associated with the processing of large volumes of liquid. EBA technology is particularly suited for the processing of mixtures, which still contain certain particulates, as well as for the processing of large volumes of liquid. In an exemplary embodiment, EBA technology is used to process cell culture liquid, e.g., immediately after harvest without prior clarification. In one embodiment EBA is used prior to CaCl₂ precipitation. In another example, EBA is used prior to hollow fiber or depth filtration. In yet another example EBA is used prior to initial viral filtration. In another embodiment, EBA is used to replace one or more of the early purification steps in the EPO purification cascade.

A variety of resin types have been developed for use with EBA technology, which are available commercially. Column materials for EBA are available, for instance from GE Healthcare (e.g., STREAMLINE products, such as Stream Line Direct 24, Big Beads SP, Capto S). Other products are available from Upfront (FastLine products). Typical adsorbents include those for anion and cation exchange chromatography, affinity chromatography and hydrophobic interaction chromatography (HIC).

### Ion Exchange Chromatography

Anion and cation exchange chromatography are known in the art. Ion exchange chromatography separates compounds based on their net charge. Ionic molecules are classified as either anions (having a negative charge) or cations (having a positive charge). Some molecules (e.g., proteins) may have both anionic and cationic group. A positively charged support (anion exchanger) will bind a compound with an overall negative charge. Conversely, a negatively charged support (cation exchanger) will bind a compound with an overall positive charge. Ion exchange matrices can be further categorized as either strong or weak exchangers. Strong ion exchange matrices are charged (ionized) across a wide range of pH levels. Weak ion exchange matrices are ionized within a narrow pH range. The ionic groups of exchange columns are covalently bound to the gel matrix and are compensated by small concentrations of counter ions, which are present in the buffer. The most common ion exchange chemistries include: quaternary ammonium residues (O) for strong anion exchange, diethylaminoethyl residues (DEAE) for weak anion exchange, sulfonic acid (S) for strong cation exchange and carboxymethyl residues (CM) for weak cation exchange.

When adding a sample to the column, an exchange with the weakly bound counter ions takes place. The size of the sample volume in ion exchange chromatography is of secondary importance as long as the initial solvent is of low eluting strength, so as not to allow the sample components to proceed through the column. Under such conditions, the sample components are preferably collected at the top of the column. When the gradient is begun with the addition of a stronger eluting mobile phase, the sample components begin their separation. If poor separation is observed, it might be improved by a change in the gradient slope. If the peptide does not bind to the column under the selected conditions, the composition and/or the pH of the starting buffer should be changed. The buffer system can further be optimized by choosing different buffer salts since each buffer composition solvates the ion exchanger and the sample components uniquely.

In general, any conventional buffer system with a salt concentration of about 5 mM up to about 50 mM can be used for ion exchange chromatography. However, positively charged buffering ions are used for anion exchangers and negatively charged ones are used for cation exchangers. Phosphate buffers are generally used on both exchanger types. Typically, the highest salt concentration that permits binding of the peptide of interest is used as the starting condition. All buffers are prepared from MilliQ-water and filtered (0.45 or 0.22 μm filter).

### Anion Exchange Chromatography

In an exemplary embodiment a sample containing the polypeptide of interest is loaded onto an anion exchanger in a loading buffer comprising a salt concentration below the concentration at which the peptide would elute from the column. In one example, the pH of the buffer is selected so that the purified peptide is retained on the anion exchange medium. Changing the pH of the buffer alters the charge of the peptide, and lowering the pH value shortens the retention time with anion exchangers. The isoelectric point (pI) of a protein is the pH at which the charge of a protein is zero. Typically, with anion exchangers the pH value of the buffer is kept 1.5 to 2 times higher than the pI value of the peptide of interest. Alternatively, the anion exchange conditions are selected to preferentially bind impurities, while the purified peptide is found in the flow-through.

The column may be washed with several column volumes (CV) of buffer to remove unbound substances and/or those substances that bind weakly to the resin. Fractions are then eluted from the column using, for example, a saline gradient according to conventional methods. The salt in the solution competes with the protein in binding to the column and the protein is released. Components with weak ionic interactions elute at a lower salt concentration than components with a strong ionic interaction. Sample fractions are collected from the column. Fractions containing high levels of the desired peptide and low levels of impurities are pooled or processed separately.

Anion exchange media are known to those of skill in the art. Exemplary anion exchange media are described, e.g., in Protein Purification Methods, A Practical Approach, Ed. Harris E L V, Angal S, IRL Press Oxford, England (1989); Protein Purification, Ed. Janson J C, Ryden L, VCH-Verlag, Weinheim, Germany (1989); Process Scale Bioseparations for the Biopharmaceutical Industry, Ed. Shukla A A, Etzel M R, Cadam S, CRC Press Taylor & Francis Group (2007), pages 188-196; Protein Purification Handbook, GE Healthcare 2007 (18-1132-29) and Protein Purification, Principles, High Resolution Methods and Applications (2^{nd} Edition 1998), Ed. Janson J-C and Ryden L, the disclosures of which are are incorporated herein by reference in their entirety. An exemplary anion exchanger of the invention is selected from quaternary ammonium resins and DEAE resins. In one embodiment, the anion exchanger is a quaternary ammonium resin (e.g. Mustang Q ion exchange membrane, Pall Corporation). Other useful resins include QXL, Capto and BigBeads resins. In one example, the anion exchanger is Sartobind Q.

Exemplary anion exchange media are summarized below:

### GE Healthcare:

### Q-Sepharose FF

### Q-Sepharose BB

### Q-Sepharose XL

### Q-Sepharose HP

### Mini Q

### Mono Q

### Mono P

### DEAE Sepharose FF

### Source 15Q

### Source 30Q

### Capto Q

ANX Sepharose 4 FF (high sub)

### Streamline DEAE

### Streamline QXL

### Applied Biosystems:

Poros HQ 10 and 20 um self pack
Poros HQ 20 and 50 um bulk media

### Poros PI 20 and 50 um

### Poros D 50 um

### Tosohaas:

### Toyopearl DEAE 650S, M and C

### Super Q 650

### QAE 550C

### Pall Corporation:

### DEAE Hyper D

### Q Ceramic Hyper D

Mustang Q membrane absorber

### Merck KGgA:

### Fractogel DMAE

### FractoPrep DEAE

### Fractoprep TMAE

### Fractogel EMD DEAE

### Fractogel EMD TMAE

Sartorious: Sartobind Q membrane absorber

The anion exchangers used in the methods of the invention are optionally membrane adsorbers rather than chromatographic resins or supports. The membrane adsorber is optionally disposable.

In one embodiment, the anion exchangers used in the process of the current invention are employed to separate the purified peptide from contaminants such as viral particles, particulates, proteins/peptides and DNA molecules. In another embodiment, anion exchange chromatography is used to remove proteases and/or endoglycosidases. In one example, sepharose Q filtration is used prior to the first capture step (e.g., dye-ligand affinity chromatography).

### Cation Exchange Chromatography

In an exemplary embodiment a sample containing the peptide of interest is loaded onto a cation exchange resin in a loading buffer comprising a salt concentration below the concentration at which the peptide would elute from the column.

In one example, the pH of the loading buffer is selected so that the peptide of interest is retained on the cation exchange resin. Changing the pH of the buffer alters the charge of the peptide and increasing the pH of the buffer shortens the retention times with cation exchangers. Typically, cation exchanges are performed at 1.5 to 2 pH units below the peptide's pI. Alternatively, the cation exchange conditions are selected to preferentially bind impurities, while the purified peptide is found in the flow-through.

In another example, the column is washed with several column volumes of buffer to remove unbound substances or those substances that bind weakly to the resin. Fractions are then eluted from the column using a salt gradient according to conventional methods. Sample fractions may be collected from the column. For example, one or more fraction containing high levels of the desired polypeptide and low levels of impurities are collected, and optionally pooled.

In an exemplary embodiment the cation exchangers used in the process of the current invention provide one of the primary purification steps of the purification process.

In one embodiment, the cation exchanger removes the majority of undesired proteins from the mixture, which contains the peptide of interest.

Cation exchange media are known to those of skill in the art. Exemplary cation exchange media are described, e.g., in Protein Purification Methods, A Practical Approach, Ed. Harris E L V, Angal S, IRL Press Oxford, England (1989); Protein Purification, Ed. Janson J C, Ryden L, VCH-Verlag, Weinheim, Germany (1989); Process Scale Bioseparations for the Biopharmaceutical Industry, Ed. Shukla A A, Etzel M R, Gadam S, CRC Press Taylor & Francis Group (2007), pages 188-196; Protein Purification Handbook, GE Healthcare 2007 (18-1132-29) and Protein Purification, Principles, High Resolution Methods and Applications (2^{nd} Edition 1998), Ed. Janson J-C and Ryden L, the disclosures of which are are incorporated herein by reference in their entirety. In an exemplary embodiment, cation exchange resins of use in the invention are selected from sulfonic acid (S) and carboxymethyl (CM) supports. In one embodiment, the cation exchanger is a sulfonic acid support (e.g. UNOsphereS, Bio-Rad Laboratories) or a sulphopropyl (SP) resin. In another embodiment, the cation exchange resin is selected from SPFF, SPHP sepharose, BigBeads SP, Capto S and the like. In one example, the cation exchanger is Source 15S.

Exemplary commercial cation exchange media are summarized below:

### GE Healthcare:

### SP-Sepharose FF

### SP-Sepharose BB

### SP-Sepharose XL

### SP-Sepharose HP

### Mini S

### Mono S

### CM Sepharose FF

### Source 15S

### Source 30S

### Capto S

### MacroCap SP

### Streamline SP-XL

### Streamline CST-1

### Tosohaas Resins:

### Toyopearl Mega Cap II SP-550 EC

### Toyopearl Giga Cap S—650M

### Toyopearl 650S, M and C

### Toyopeal SP650S, M, and C

### Toyopeal SP550C

### JT Baker Resins:

### Carboxy-Sulphon—5, 15 and 40 um

### Sulfonic—5, 15, and 40 um

### Applied Biosystems:

### Poros HS 20 and 50 um

### Poros S 10 and 20 um

### Pall Corp:

### S Ceramic Hyper D

### CM Ceramic Hyper D

### Merck KGGA Resins:

### Fractogel EMD SO3

### Fractogel EMD COO—

### Fractogel EMD SE Hicap

### Fracto Prep SO3

### Biorad Resin:

### Unosphere S

### Sartorius Membrane:

Sartobind S membrane absorber

The cation exchangers used in the methods of the invention are optionally membrane adsorbers rather than chromatographic resins or supports. In an exemplary embodiment the membrane adsorber is a sulfonic acid (S) cation exchanger (e.g. Sartobind S, Sartorius A G). The membrane adsorber is optionally disposable.

The ion exchangers used in the methods of the invention are optionally membrane adsorbers rather than chromatographic resins or supports. In an exemplary embodiment, the membrane adsorber is a cation exchanger. In another exemplary embodiment the membrane adsorber is a sulfonic acid (S) cation exchanger (e.g. SartobindS, Sartorius A G). The membrane adsorber is optionally disposable.

### Hydrophobic Interaction Chromatography (HIC)

Hydrophobic interaction chromatography (HIC) is a liquid chromatography technique that separates biomolecules based on differences in their surface hydrophobicity. Hydrophobic amino acids exposed on the surface of a polypeptide, can interact with hydrophobic moieties on the HIC matrix. The amount of exposed hydrophobic amino acids differs between polypeptides and so does the ability of polypeptides to interact with HIC gels. Hydrophobic interaction between a biomolecule and the HIC matrix is enhanced by high ionic strength buffers. and HIC of biomolecules is typically performed at high salt concentrations. The elution of the peptide of interest from the column is then initiated by decreasing salt gradients.

In one embodiment, HIC is used to avoid other forms of hydrophobic chromatography, such as reverse-phase chromatography. While reverse-phase (RP) chromatography can be used to purify polypeptides, the technique is not desirable because it typically requires the use of water-soluble organic solvents, such as acetonitrile or alcohols. Organic solvents, especially in large-scale processes are not only associated with environmental concerns, but can also effect the chemical stability of the purified polypeptide. Therefore, process steps that rely on aqueous solutions are generally preferred. Hence, in one embodiment, the current invention provides methods that do not utilize reverse phase chromatography. In another embodiment, the method of the invention allows for the isolation of polypeptides essentially without the use of organic solvents, such as ethanol, propanol and acetonitrile.

Exemplary HIC resins useful in the methods of the invention are described, e.g., in Protein Purification Methods, A Practical Approach, Ed. Harris E L V., Angal S, IRL Press Oxford, England (1989) p. 224 and Protein Purfication, Ed. Janson J C, Ryden L, VCH-Verlag, Weinheim, Germany (1989) pp. 207-226. HIC media are distinguished by the hydrophobic moiety that they carry, by the particle size (e.g. bead size), and the density of the hydrophobic moieties on the HIC matrix (e.g. low substitution or high substitution). In an exemplary embodiment, the hydrophobic moieties of the column matrix are members selected from alkyl groups, aromatic groups and ethers. Exemplary hydrophobic alkyl groups include lower alkyl groups, such as n-propyl, isopropyl, n-butyl, iso-butyl, and n-octyl. Exemplary aromatic groups include substituted and unsubstituted phenyl.

Exemplary HIC resins useful in the methods of the invention are described, e.g., in Protein Purification Methods, A Practical Approach, Ed. Harris E L V, Angal S, IRL Press Oxford, England (1989) page 224, Protein Purification, Ed. Janson J C, Ryden L, VCH-Verlag, Weinheim, Germany (1989) pages 207-226, Process Scale Bioseparations for the Biopharmaceutical Industry, Ed. Shukla A A, Etzel M R, Gadam S, CRC Press Taylor & Francis Group (2007), pages 197-206, Hydrophobic Interaction and Reversed Phase Chromatography, Principles and Methods, GE Healthcare 2007 (11-0012-69), Protein Purification Handbook, GE Healthcare 2007 (18-1132-29) and Protein Purification, Principles, High Resolution Methods and Applications (2^{nd} Edition 1998), Ed. Janson J-C and Ryden L, “Hydrophobic Interaction Chromatography, page 283, the disclosures of which are incorporated herein by reference in their entirety.

HIC media are distinguished by the hydrophobic moiety that they carry, by the particle size (e.g. bead size), the pore size and the density of the hydrophobic moieties on the HIC matrix (e.g. low substitution or high substitution). In an exemplary embodiment, the hydrophobic moieties of the column matrix are members selected from alkyl groups, aromatic groups and ethers. Exemplary hydrophobic alkyl groups include lower alkyl groups, such as n-propyl, isopropyl, n-butyl, iso-butyl, and n-octyl. Exemplary aromatic groups include substituted and unsubstituted phenyl.

In another exemplary embodiment the matrix of the HIC medium is a member selected from agarose, sepharose (GE Healthcare), polystyrene, divinylbenzene, and combinations thereof. Exemplary HIC resins include Butyl Fast Flow and Phenyl Fast Flow (e.g., GE Healthcare) in either low or high substituted versions. In one embodiment, the HIC resin is a phenyl resin. In one particular example, the HIC resin is Phenyl650S or Phenyl650M (e.g., Tosohaas, Toyopearl). In another embodiment, the HIC resin is a butyl resin, such as Butyl Sepharose Fast Flow (GE Healthcare).

In one example, the HIC medium is selected from the following commercial resins:

### GE Healthcare HIC Resins:

### Butyl Sepharose 4 FF

### Butyl-S Sepharose FE

### Octyl Sepharose 4 FF

### Phenyl Sepharose BB

### Phenyl Sepharose HP

### Phenyl Sepharose 6 FF High Sub

### Phenyl Sepharose 6 FF Low Sub

### Source 15ETH

### Source 15ISO

### Source 15PHE

Capto Phenyl (prototype resin)
Capto Butyl (prototype resin)

### Streamline Phenyl

### Tosohaas HIC Resins:

### TSK Ether 5PW (20 um and 30 um)

### TSK Phenyl 5PW (20 um and 30 um)

### Phenyl 650S, M, and C

### Butyl 650S, M and C

### Hexyl-650M and C

### Ether-650S and M

### Butyl-600M

### Super Butyl-550C

### PPG-600M

### Waters HIC Resins:

YMC-Pack Octyl Columns-3, 5, 10P, 15 and 25 um with pore sizes 120, 200, 300A
YMC-Pack Phenyl Columns-3, 5, 10P, 15 and 25 um with pore sizes 120, 200 and 300A
YMC-Pack Butyl Columns-3, 5, 10P, 15 and 25 um with pore sizes 120, 200 and 300A

### CHISSO Corporation HIC Resins:

### Cellufine Butyl

### Cellufine Octyl

### Cellufine Phenyl

### JT Baker HIC Resin:

### WP HI-Propyl (C3)

### Biorad HIC Resins:

Macroprep t-Butyl
Macroprep methyl

### Applied Biosystems HIC Resin:

### High Density Phenyl 13 HP2 20 um

In another exemplary embodiment, the buffer in which the product is applied to the HIC column contains salts, such as sodium acetate (NaOAc), sodium chloride (NaCl), and sodium sulfate (Na₂SO₄). The concentration ranges for these and other salts are generally optimized for each type of HIC resin to affect optimal binding of the peptide.

In an exemplary embodiment, the concentration of sodium sulfate in the loading buffer is about 100 mM to about 1M, preferably about 300 mM to about 800 mM and, more preferably, about 400 mM to about 600 mM. In another exemplary embodiment, the concentration of NaCl in the buffer is about 10 mM to about 1M, preferably about 200 mM to about 400 mM and, more preferably, about 200 mM to about 300 mM. In yet another exemplary embodiment the concentration of NaOAc in the loading buffer is about 1 mM to about 50 mM, preferably about 5 mM to about 20 mM and, more preferably, about 5 mM to about 15 mM.

In another exemplary embodiment, the buffer in which the product is applied to the HIC column has a pH of about 4.0 to about 6.0, preferably about 4.5 to about 5.5 and, more preferably, about 5.0.

In yet another exemplary embodiment, the product is eluted from the HIC resin with a sodium acetate buffer at a pH of about 5.0 to about 7.5. Exemplary elution buffer systems include TRIS buffer and HEPES buffer. Optionally, the elution buffer does not contain sodium sulfate. In a further exemplary embodiment the elution buffer contains ethanol in an amount of about 5% to about 10% v/v.

In one aspect, the method of the invention includes separating the polypeptide from an impurity, wherein the impurity has a molecular weight smaller than the polypeptide by hydrophobic interaction chromatography. The method comprises: (a) applying a mixture containing the polypeptide and the impurity to a suitable hydrophobic interaction chromatography resin; (b) eluting the impurity from the resin; (c) cluting the peptide from the resin; and collecting one or more eluate fraction containing the polypeptide.

In one preferred embodiment, HIC is employed as an orthogonal method of purification to remove impurities that are difficult to remove using other means, and preferably those that have a smaller molecular weight than the peptide being purified.

In an exemplary embodiment, EPO polypeptide is isolated from a low-molecular weight impurity using HIC. For example, the content of a low-molecular weight impurity in an EPO peptide solution is reduced by at least 50% of its content before HIC. In another exemplary embodiment, the impurity is reduced by at least 60%, preferably at least 80% and, more preferably, at least 90% of its original content. In certain preferred embodiments the content of the low-molecular weight impurity in the mixture processed by HIC is reduced by at least 91%, at least 92%, at least 93%, at least 94%, at least 95%, at least 96%, at least 97%, at least 98% or at least 99%.

In an exemplary embodiment, HIC chromatography is employed to reduce the content of low-molecular weight impurities in a polypeptide solution. In one embodiment, during HIC chromatography, low-molecular weight impurities are found in the flow through, while the polypeptide (e.g., EPO) is initially retained on the HIC column.

In an exemplary embodiment, HIC is performed subsequent to hydroxyapatite (HA) or fluoroapatite chromatography. Performing the two chromatographic steps in this order increases the recovery of peptide after HIC and requires limited conditioning of the buffer system prior to HIC. In an exemplary embodiment, the pH of the hydroxyapatite product pool is lowered to about 5.0 to about 5.5 by addition of an organic acid (e.g. acetic acid). Sodium sulfate can then be added to a concentration of about 500 mM to about 1.0 M, preferably about 500 mM in order to condition the partially purified peptide for hydrophobic interaction chromatography. In another example, HIC is performed prior to cation exchange and/or HA or fluoroapatite chromatography.

### Mixed-Mode Chromatography

In an exemplary embodiment, the peptide purification process of the invention includes a mixed-mode chromatography step. Mixed-mode media, which may also be referred to as “multi-modal”, are known to those of skill in the art. See, e.g., Process Scale Bioseparations for the Biopharmaceutical Industry, Ed. Shukla A A, Etzel M R, Gadam S, CRC Press Taylor & Francis Group (2007), page 218, which is incorporated herein by reference. Certain dye-ligand affinity resins may also be considered mixed-mode resins (e.g., providing anion exchange and hydrophobic interaction capabilities). For the purpose of this application, mixed-mode chromatography also includes hydroxyapatite and fluoroapatite chromatography, which are described in more detail, below. Exemplary mixed-mode resins are summarized below:

### GE Healthcare:

### Capto MMC

### Capto Adhere

### Blue Sepharose FF

### Blue Sepharose HP

### Capto Blue

### IgM HP

### IgY HP

### Pall Life Sciences:

### BioSepra HEA HyperCel

### BioSepra PPA HyperCel

### BioSepra MEP HyperCel

### HA Ultrogel Hydroxyapatite

### BioRad:

### Hydroxyapatite Type I and II

### Fluoroapatite Type I and II

### Tosohass:

### Toyopearl AF Blue HC 650

### Toyopearl AF Red HC 650

In one example, the mixed-mode media employs anion exchange or cation exchange (ionic interaction) capabilities in combination with additional modalities, such as hydrophobic interaction capabilities and/or hydrogen-bonding capabilities.

In one example, the mixed-mode medium is an anion exchanger also featuring hydrophobic interaction capabilities. In one example, the hydrophobic interaction capabilities of the mixed-mode medium are due to the presence of at least one “hydrophobic moiety”. Exemplary hydrophobic moieties include linear or branched alkyl groups, aryl or heteroaryl groups, which are preferably not substituted with polar substituents (e.g., hydroxyl groups) but may be substituted with other alkyl groups. In one example, the mixed-mode medium includes a mixed-mode ligand having a hydrophobic moiety that includes at least 3, at least 4, at least 5 or at least 6 carbon atoms. In another example, the hydrophobic moiety of the mixed-mode ligand includes at least 7, at least 8, at least 9 or at least 10 carbon atoms.

The carbon atoms of the hydrophobic moiety may be arranged in a straight or branched chain or may be arranged to form a cycloalkyl or aromatic (e.g., phenyl) ring structure. Alternatively, the mixed-mode ligand includes a hydrophobic moiety that is a combination of at least one straight or branched carbon chain (e.g., —CH₂—, —CH₂CH₂—, CH₂CH₂CH₂—) and at least one ring structure (e.g., an aryl, a heteroaryl, or a cycloalkyl moiety). In a further example, the hydrophobic moiety includes an n-alkyl group (e.g., —CH₂—, —CH₂CH₂—, CH₂CH₂CH₂—) substituted with an aryl or heteroaryl moiety. In a particular example, the hydrophobic moiety is a phenyl-substituted methyl-, ethyl-, n-propyl- or n-butyl group.

In one example, the mixed-mode medium is an anion exchanger having hydrogen bonding capabilities. Hydrogen bonding capabilities may be provided by incorporating into the mixed-mode ligand at least one moiety that includes a hydroxyl group (e.g., hydroxyethyl, hydroxypropyl or hydroxybutyl group).

In another embodiment, the mixed-mode ligand is an anion exchanger having hydrogen bonding capabilities and hydrophobic interaction capabilities. An exemplary multi-modal medium according to this embodiment is Capto Adhere, a resin currently available from GE Healthcare. In another embodiment, the mixed-mode medium is used in such a way that the purified polypeptide is found in the flow-through while certain impurities are retained by the mixed-mode medium and thus separated from the polypeptide.

In another example, the mixed-mode medium is a cation exchanger also featuring hydrophobic interaction capabilities. In yet another example, the mixed-mode medium is a cation exchanger also featuring hydrophobic interaction capabilities as well as hydrogen-bonding capabilities. Chemical moieties providing hydrophobic interaction and hydrogen-bonding capabilities are discussed herein, above, and are equally applicable to the examples in this paragraph.

In yet another embodiment, mixed-mode chromatography is used to remove proteases, such as and/or endoglycanases from a polypeptide solution obtained from insect cell culture. In a particular example, mixed-mode chromatography is used after the culture broth is cleared of particulates, such as cellular debris (e.g., using depth filtration or hollow fiber filtration) and prior to the polypeptide capture step, which may employ a HIC or a Cibacron Blue resin). In another example, mixed-mode chromatography is used after (e.g., immediately after) the capture step. Generally, it is preferred that the mixed-mode step be performed early in the purification process in order to minimize loss of polypeptide due to enzymatic degradation.

### Hydroxyapatite and Fluoroapatite Chromatography

In an exemplary embodiment, the peptide purification process of the invention includes mixed-mode or pseudo-affinity chromatography, such as chromatography performed on ceramic or crystalline apatite media, such as hydroxyapatite (HA) chromatography and fluoroapatite (FA) chromatography. HA and FA chromatography are effective purification mechanisms, providing biomolecule selectivity, complementary to ion exchange or hydrophobic interaction techniques. Hydroxyapatite and fluoroapatite chromatography are known in the art.

### Hydroxyapatite

Exemplary hydroxyapatite sorbents are selected from ceramic and crystalline materials. Ceramic hydroxyapatite sorbents are available in different particle sizes (e.g. type 1, Bio-Rad Laboratories). In an exemplary embodiment the particle size of the ceramic hydroxyapatite sorbent is between about 20 μm and about 180 μm, preferably about 60 to about 100 μm, and, more preferably about 80 μm.

In one embodiment, the hydroxyapatite sorbent is composed of cross-linked agarose beads with microcrystals of hydroxyapatite entrapped in the agarose mesh. Optionally, the agarose is chemically stabilized (e.g. with epichlorohydrin under strongly alkaline conditions). In one exemplary embodiment, the hydroxyapatite sorbent is HA Ultrogel (Pall Corporation).

### Fluoroapatite

Exemplary fluoroapatite sorbents are selected from ceramic (e.g., bead-like particles) and crystalline materials. Ceramic fluoroapatite sorbents are available in different particle sizes (e.g. type 1 and type 2, Bio-Rad Laboratories). In an exemplary embodiment the particle size of the ceramic fluoroapatite sorbent is from about 20 μm to about 180 μm, preferably about 20 to about 100 μm, more preferably about 20 μm to about 80 μm. In one example, the particle size of the ceramic fluoroapatite medium is about 40 μm (e.g., type 1 ceramic fluoroapatite). In another example, the fluoroapatite medium includes hydroxyapatite in addition to fluoroapatite. In a particular example, the fluoroapatite medium is Bio-Rad's CFT™ Ceramic Fluoroapatite.

The selection of the flow velocity used for loading the sample onto the hydroxyapatite or fluoroapatite column, as well as the elution flow velocity depends on the type of hydroxyapatite or fluoroapatite sorbent and on the column geometry. In one exemplary embodiment, at process scale, the loading flow velocity is selected from about 30 to about 900 cm/h, from about 150 to about 900 cm/h, preferably from about 500 to about 900 cm/h and, more preferably, from about 600 to about 900 cm/h.

In an exemplary embodiment, the pH of the elution buffer is selected from about pH 7 to about pH 9, and preferably from about pH 7.5 to about pH 8.0.

In one aspect the present invention provides a method of purifying a recombinant peptide by hydroxyapatite or fluoroapatite chromatography. The method includes the following steps: (a) desalting a mixture containing the peptide, forming a desalted mixture (e.g. by gel filtration) that has a salt conductivity, which is sufficiently low to increase the peptide-binding capacity of the hydroxyapatit or fluoroapatite resin; (b) applying the desalted mixture to a hydroxyapatite or fluoroapatite resin; (c) washing the hydroxyapatite or fluoroapatite resin, thereby eluting unwanted components from the resin; (d) eluting the peptide from the resin with an elution buffer that optionally contains an amino acid; and (e) collecting one or more eluate fraction containing the peptide.

### Desalting

In one embodiment, the mixture containing the peptide of interest is desalted prior to subjecting the mixture to HA or fluoroapatite chromatography. The desalting step increases the capacity of the apatite column to bind the peptide of interest. In one embodiment, the apatite column capacity (amount of peptide per liter of resin), increases with decreasing salt conductivity of the load, which contains the peptide.

In an exemplary embodiment, in which the load is desalted, the mass loading of peptide per liter of HA resin is from about 1 to about 25 g/L, from about 1 to about 20 g/L, preferably from about 1 to about 15 g/L and more preferably from about 1 to about 10 g/L.

In an exemplary embodiment, in which the peptide being purified is EPO, desalting the loading buffer increases the HA column capacity. In an exemplary embodiment, the peptide-binding capacity, at which the breakthrough of EPO peptide is less than 10%, is at least about 2 g/L, at least about 4 g/L, at least about 6 g/L, at least about 8 g/L and preferably at least about 10 g/L.

In another exemplary embodiment, the conductivity of the load can be decreased using a method selected from desalting and diluting.

In an exemplary embodiment, the conductivity of the loading buffer is lowered by desalting and preferred conductivities are from about 0.1 to about 4.0 mS/cm, preferably from about 0.1 to about 1.0 mS/cm, more preferably from about 0.1 to about 0.6 mS/cm and, still more preferably, from about 0.1 to about 0.4 mS/cm.

Desalting of peptide solutions is achieved using membrane filters wherein the membrane filter has a MWCO smaller than the peptide/protein of interest. The peptide/protein is found in the retentate and is reconstituted in a buffer of choice. However, when purifying peptides of relatively low molecular weight (e.g. EPO), the MWCO of the membrane used for desalting must be relatively small in order to avoid leaking of the peptide through the membrane pores. However, filtering a large volume of liquid through a small MWCO membrane (e.g. with a pore size of about 5 kDa), typically requires large membrane areas and the filtering process is time consuming.

Therefore, in one embodiment, desalting of the HA or fluoroapatite chromatography load is accomplished using size-exclusion chromatography (e.g. gel filtration). The technique separates molecules on the basis of size. Typically, high molecular weight components can travel through the column more easily than smaller molecules, since their size prevents them from entering bead pores. Accordingly, low-molecular weight components take longer to pass through the column. Thus, low molecular weight materials. such as unwanted salts, can be separated from the peptide of interest.

In an exemplary embodiment, the column material is selected from dextran, agarose, and polyacrylamide gels, in which the gets are characterized by different particle sizes. In another exemplary embodiment, the material is selected from rigid, aqueous-compatible size exclusion materials. An exemplary gel filtration resin of the invention is Sepharose G-25 resin (GE Healthcare).

In an exemplary embodiment, desalting is performed subsequent to cation exchange chromatography (e.g. after UnoSphere S chromatography).

### Addition of an Amino Acid to the Elution Buffer

In one embodiment, an amino acid is added to the elution buffer, which is used to elute the polypeptide of interest from a chromatography medium, such as a mixed-mode or dye-ligand affinity chromatography medium, or a HA or fluoroapatite resin. In an exemplary embodiment the amino acid is added to the elution buffer at a final concentration of about 5 mM to about 50 mM, about 10 mM to about 40 mM, preferably about 15 mM to about 30 mM and, more preferably, about 20 mM.

In one embodiment, the addition of an amino acid (e.g. glycine or arginine) to the elution buffer increases the step recovery of peptide from HA chromatography when compared to the recovery obtained without the addition of an amino acid. In an exemplary embodiment, the recovery of peptide is increased by addition of the amino acid at least about 1% to about 20%, by at least about 1% to about 15%, by at least about 1% to about 10%, preferably by at least about 1% to about 7% and, more preferably, by about 5%.

In another exemplary embodiment, the addition of an amino acid (e.g. glycine) causes the elution peak of the purified peptide to be sharper. Thus, less peptide is recovered in the tail fractions of the peak and more peptide is recovered in the main peak.

In another exemplary embodiment, the addition of an amino acid (e.g. glycine) does not decrease the purity of the product from HA chromatography.

In an exemplary embodiment, the amino acid is glycine. In a preferred embodiment, glycine is added to the elution buffer at a final concentration of 20 mM.

### Dye-Ligand Affinity Chromatography

In one embodiment, the method of the invention includes at least one polypeptide capture step, in which the desired polypeptide (e.g., EPO, or ST6GalNAc1) binds to a separation medium, while impurities are found in the flow-trough. Exemplary capture steps may involve HIC or dye-ligand affinity chromatography, such as chromatography on Cibacron Blue resins. Preferred media useful for polypeptide capture include those that allow for good recovery of polypeptide (e.g., greater than 80%) and suitable overall binding capacity for the desired polypeptide.

In a preferred embodiment, the capture step employs dye-ligand affinity chromatography. Dye-ligand affinity chromatography media are known to those of skill in the art. A typical dye-ligand affinity resin includes a dye ligand bound to a support matrix. In one embodiment, the dye-ligand includes at least one molecule of Cibacron Blue (CB). Exemplary cibacron blue dyes include several isomers with respect to the position of a sulfonate group on the terminal phenyl ring of the molecule. For example, while Cibacron Blue F3GA represents a mixture of meta- and para-isomers, the ortho-isomer has been named Cibacron Blue 3GA. All such isomers are useful within the methods of the invention. In one example, the free dye or a derivative thereof (e.g., Blue Dextran) is covalently linked to a solid support, such as a Sepharose, a Sephadex or a polyacrylamide matrix. Exemplary dye-ligand affinity resins are discussed in Subramanian S, CRC Critical Reviews in Biochemistry 1984, 16(2): 169-205, which is incorporated herein by reference in its entirety.

In an exemplary embodiment, dye-ligand affinity chromatography is used subsequent to mixed-mode chromatography as described above. For example, mixed-mode and Cibacron Blue modules are combined to a continuous-mode unit.

### III. e) Viral Inactivation

The peptide purification process of the current invention includes one or more viral inactivation steps in order to inactivate enveloped and non-enveloped virus particles that may be present in the mixture. This is particularly important when the final product is intended for use in living organisms. Pathogenic viruses are removed to render the product safe for use in humans. Removal of virus particles may be accomplished using a combination of filtration and chromatographic steps. Inactivation of enveloped viruses may be accomplished chemically, e.g. by addition of a detergent. Inactivation of remaining viruses may be accomplished through a low pH hold procedure. Viruses may also be inactivated using irradiation of the polypeptide solution with light (e.g., UV light). Methods to inactivate viruses using UV light (e.g., UVC light) are known in the art (e.g., those employed by the UVivatec®-System (Bayer Technology Services).

Viral Inactivation Using a Detergent In one exemplary embodiment viral inactivation involves the addition of a detergent to the partially purified peptide solution. In an exemplary embodiment, the detergent is TritonX (e.g. TritonX-100). In a further exemplary embodiment, TritonX-100 is added to inactivate enveloped viruses.

In another exemplary embodiment, the detergent is added at a final concentration of about 0.01% to about 0.1% v/v, preferably about 0.04% to about 0.06% v/v, and, more preferably at a final concentration of about 0.05% v/v. In one exemplary embodiment the detergent is added to the partially purified peptide solution after purification by anion exchange chromatography (e.g. Mustang Q).

### Viral Inactivation by a Low-pH Hold Procedure

It is known in the art that many viruses do not survive a prolonged treatment with a low pH medium. However, when purifying peptides and proteins, the pH of the buffer system is generally crucial in maintaining the stability of the product. Many proteins and peptides cannot withstand a pH well below 7.0.

In one aspect, the present invention provides a method of inactivating viruses in a mixture containing the peptide of interest. The method comprises: (a) lowering the pH of the mixture containing the peptide to a pH below pH 7; (b) maintaining the low pH of step (a) for a selected period of time (e.g. about 1 hour); and raising the pH of the mixture containing the peptide to a pH suitable for further processing.

In an exemplary embodiment, the pH of step (a) is lowered to about pH 2 to about pH 4, preferably to about pH 2 to about pH 3 and, more preferably, to about pH 2 to about pH 2.5. In one preferred embodiment, the pH of the product solution is lowered to between about pH 2.2 to about pH 2.5.

In a further exemplary embodiment, the pH of the peptide solution is maintained at the low pH (e.g. about pH 2.2) for at least about 30 min to at least about 2 hours, preferably at least about 1 hour, before the pH is raised.

In another exemplary embodiment, the pH of the product solution is lowered while the peptide solution has controlled room temperature.

In one exemplary embodiment, the pH of the peptide solution is adjusted using acids, which are suitable for biological applications. Exemplary acids include organic acids, inorganic acids and combinations thereof. In an exemplary embodiment the organic acid is a member selected from acetic acid, citric acid, lactic acid, oxalic acid and succinic acid. In another exemplary embodiment the inorganic acid is a member selected from hydrochloric acid (HCl) and phosphoric acid (H₃PO₄).

### III. f) Inactivation of Proteases and Glycosidases

In one embodiment, a protease inhibitor, e.g., methylsulfonylfluoride (PMSF), or sodium citrate is added to the partially purified peptide solution to inhibit proteolysis. In another embodiment, a glycosidase inhibitor may be added. This step protects the peptide of interest from degradation. This is particularly useful if the partially purified peptide solution is stored prior to further processing. Antibiotics are optionally added to prevent the growth of adventitious contaminants.

### III. g) Viral Clearance and Storage

In an exemplary embodiment, the peptide purification process of the current invention includes an additional ultrafiltration step to affect viral clearance. Typically, this step occurs towards the end of the purification process and employs a membrane with a MWCO larger than the peptide of interest to allow the peptide to flow through the membrane. In an exemplary embodiment, this viral clearance step is introduced into the process after purification of the product by chromatographic means. A number of ultrafiltration membranes are available that are recommended for viral removal. In an exemplary embodiment the membrane is NFP membrane (Millipore Corporation). In one embodiment NFP filtration is performed after HIC chromatography and prior to final diafiltration/ultrafiltration.

In another exemplary embodiment, the peptide purification process of the present invention includes a diafiltration step towards the end of the process. In an exemplary embodiment the diafiltration step is employed to concentrate the sample. In another exemplary embodiment the diafiltration step is employed to alter the buffer. In yet another exemplary embodiment, the new buffer is suitable for storage of the product. In another exemplary embodiment, the diafiltration membrane has a MWCO of about 4 kDa to about 10 kDa, preferably about 4 kDa to about 6 kDa and, more preferably about 5 kDa.

The purified product is stored at a low temperature. In an exemplary embodiment the product is stored at about −20° C. at a peptide concentration of about 1 mg to about 2 mg of peptide per mL storage buffer.

### III. h) Exemplary Purification Process

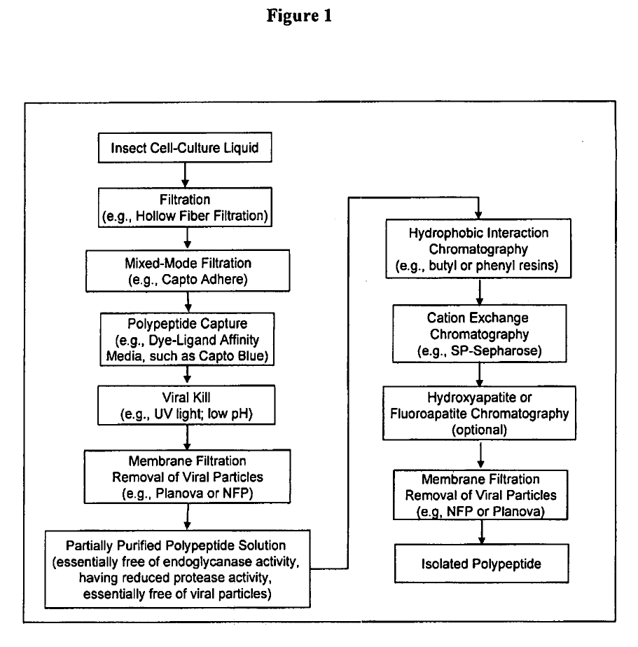

In one aspect, the invention provides a method of isolating a polypeptide (e.g., EPO) from an insect cell culture liquid (fermentation broth). An exemplary method is outlined in FIG. 1. In a first step, cells and cell debris are removed from the cell culture liquid using depth filtration or hollow-fiber filtration. In one example, the hollow fiber material has a pore size of about 0.45 μm. In one example, the hollow-fiber filtrate is diluted with water (e.g., 1:1 or 1:2). In another example, the pre-cleared solution is filtered through a membrane filter to further reduce turbidity (e.g., 0.2 μm filter membrane). In yet another example, the hollow-fiber filtrate is diafiltered, for example to condition the polypeptide solution for subsequent purification steps.

After one or more of the above described filtration steps, the resulting material is subjected to a polypeptide capture step utilizing a combination of mixed-mode chromatography and dye-ligand affinity chromatography. Exemplary mixed-mode media (e.g., Capto Adhere) and dye-ligand affinity chromatography media (e.g., Capto Blue) are described herein above. In one example, the polypeptide is found in the flow-trough of the Capto Adhere step. In another example, the flow-through of the mixed-mode step is contacted essentially immediately with a dye-ligand affinity medium. In a particular example, mixed-mode and dye-ligand affinity steps are combined in a continuous flow assembly, wherein liquid enters the dye-ligand affinity medium as soon as it exits the mixed-mode medium. In one example the polypeptide of interest is retained by the dye-ligand affinity medium and is subsequently eluted using a suitable elution buffer. In one example, the elution buffer includes potassium chloride (e.g., 2M KCl).

The resulting mixture containing the polypeptide is then irradiated with UV light or subjected to a low pH hold procedure to effect viral inactivation. In one example, the pH of the polypeptide solution is lowered to between about 3.5 and 2.0. In another example, the pH is kept below pH 3 for between about 30 min and about two hours before the pH is raised to above 4.0. The polypeptide solution is then filtered through a membrane that is suitable for the removal of viral particles. Such membranes are known in the art. Exemplary viral filters include Millipore filtration membranes (e.g., Viresolve NFP), Sartorius viral clearance filters (e.g., Virosart CPV) and Planova filters (e.g., 15N, 20N, 35N and 75N).

In one example, the resulting solution is conditioned for and subjected to hydrophobic interaction chromatography (HIC). The eluate pool from the HIC column is then subjected to cation exchange chromatography, utilizing, for example, a sulphopropyl (SP) resin (e.g., SP-Sepharose). Optionally, the resulting mixture is subjected to fluoroapatite or hydroxyapatite chromatography. For example, the mixture may be desalted using a size exclusion column (e.g. G25) to lower the salt conductivity of the peptide solution in preparation for hydroxyapatite (HA) or fluoroapatite chromatography. The desalted mixture is then loaded onto an apatite column. The elution pool from the apatite column is then optionally filtered through a suitable membrane (such as a NFP membrane) for additional viral clearance. The product may then be diafiltered, for example, across a 5 kDa membrane, and the retentate may be reconstituted in a storage buffer to reach a desired polypeptide concentration (e.g. 1-2 mg/mL).

In an exemplary embodiment according to this aspect, the peptide is produced by expression in an insect cell culture using a baculovirus expression vector system.

In another exemplary embodiment, the recombinant peptide being purified by the above described process is EPO.

### IV. Glycoconjugation

### Glycan Remodeling

After isolation of the polypeptide from the insect cell culture, the polypeptide may be modified. For example, the polypeptide may be modified through glycan remodeling, e.g., to include a substantially uniform (e.g., insect-specific) glycosylation pattern. The glycosylation pattern of the peptides can be elaborated, trimmed back or otherwise modified by methods utilizing enzymes. Methods of remodeling polypeptides using enzymes that transfer a sugar donor to an acceptor are discussed in detail in WO 03/031464 to De Frees et at (published Apr. 17, 2003); U.S. Patent Application 20040137557 (filed Nov. 5, 2002); U.S. Patent Application 20050143292 (filed Nov. 24, 2004) and WO 05/051327 (filed Nov. 24, 2004), each of which is incorporated herein by reference in its entirety.

Hence, in one embodiment, the method of the invention may further include: contacting the isolated polypeptide and a glycosyl donor molecule (e.g., a nucleotide sugar) in the presence of an enzyme for which the glycosyl donor molecule is a substrate, under conditions sufficient for the enzyme to form a covalent bond between a glycosyl moiety of the glycosyl donor molecule and the polypeptide. The polypeptide used as a substrate in this reaction may be glycosylated or non-glycosylated. The enzyme may be a glycosyltransferase, such as a GlcNAc-transferase, a GalNAc-transferase, a Gal-transferase or a sialyltransferase. In one example, the enzyme transfers a glycosyl moiety to another glycosyl moiety covalently bound to the polypeptide. In another example, the enzyme transfers the glycosyl moity onto an amino acid residue of the polypeptide.

In one example, the method of the invention includes: contacting the polypeptide, which may be glycosylated or non-glycosylated, and a nucleotide-N-acetylglucosamine (GlcNAc) or a nucleotide-N-acetylgalactosamine (GalNAc) molecule in the presence of a N-acetylglucosamine transferase (e.g., GnT1 or GnT2) or a N-acetylgalactosamine transferase, respectively. The reaction mixture may further include a nucleotide galactose (Gal) molecule, and a galactosyl transferase (e.g., GalT1). The components of the reaction mixture are contacted (e.g., in a single reaction vessel or sequentially) under conditions sufficient for the N-acetylglucosamine transferase and the galactosyl transferase to form a glycosylated polypeptide having at least one glycan residue with a terminal -GlcNAc-Gal moiety or a -GalNAc-Gal moiety. In one embodiment, the -GlcNAc-Gal moiety is added to a mannose residue, which is part of a tri-mannosyl motif. The resulting glycan residue is preferably mono-antennary with respect to the newly added -GlcNAc-Gal or -GalNAc-Gal moiety. In another embodiment, the -GalNAc-Gal moiety is added to a serine or threonine residue of the polypeptide. In one example according to any of the above embodiments, the polypeptide is EPO.

### Conjugation of the Polypeptide to a Modifying Group

In one embodiment, the method of the invention further includes covalently linking the polypeptide to a modifying group, such as a polymer. In one example, the polypeptide conjugate is formed using a chemical conjugation reaction (e.g., a chemical PEGylation reaction). Such polypeptide modifications are known in the art. In another example, the polypeptide conjugate is formed using an enzymatically catalyzed glycoconjugation reaction, during which a modified glycosyl moiety [e.g., a glycosyl moiety modified with at least one poly(alkylene oxide) moiety] is covalently linked to the polypeptide. Hence, in one example according to any of the above embodiments, the method of the invention may further include: contacting the polypeptide and a modified glycosyl donor species (e.g., a modified sugar nucleotide) having a glycosyl moiety covalently linked to a polymer (e.g., a poly(alkylene oxide) moiety), in the presence of an enzyme (e.g., a glycosyltransferase), for which the modified glycosyl donor species is a substrate, under conditions sufficient for the enzyme to catalyze the formation of a covalent bond between the glycosyl moiety that is linked to the polymer and the polypeptide. In one example, the modified glycosyl moiety is a sialic acid (SA) moiety. In another example, the enzyme is a sialyltransferase. In another example, the polymer is PEG (e.g., m-PEG). GlycoPEGylation methods are art-recognized; see for example, WO 03/031464 to DeFrees et al. or WO 04/99231 to DeFrees et al., the disclosures of which are incorporated herein by reference in their entirety.

### V. Methods of Treatment

In another aspect, the invention provides methods of treatment utilizing a composition made by a method of the invention (e.g., an isolated polypeptide or polypeptide conjugate) or a pharmaceutical formulation of the invention. In one embodiment, the invention provides a method of treating a condition in a subject in need thereof, the condition characterized by compromised red blood cell production in the subject, the method comprising: administering to the subject an amount of a composition or pharmaceutical formulation of the invention, effective to ameliorate the condition in the subject. In one example, the subject is a mammal, such as a human. In another example, the composition or formulation includes an EPO polypeptide or EPO conjugate made by a method of the invention.

In another embodiment, the invention provides a method of treating a tissue injury in a subject in need thereof. In one example, the tissue injury is caused by at least one of ischemia, trauma, inflammation and contact with a toxic substance. The method includes: administering to a subject an amount of a composition or pharmaceutical formulation of the invention that is effective in ameliorating the damage associated with the tissue injury. In one example, the subject is a mammal, such as a human. In one example, the composition includes an EPO polypeptide or an EPO polypeptide conjugate made by a method of the invention.

In another embodiment, the invention provides a method of enhancing red blood cell production in a mammal. The method includes administering to the mammal a composition or a pharmaceutical formulation of the invention. In one example, the mammal is a human. In another example, the composition or formulation includes an EPO polypeptide or an EPO polypeptide conjugate made by a method of the invention.

In another embodiment, the invention provides a method of treating anemia. The method includes administering a composition or pharmaceutical formulation of the invention to a subject in need thereof. In one example, the anemia is selected from age related anemia, early anemia of prematurity, anemia associated with chronic renal failure, anemia associated with cancer chemotherapy treatment, anemia associated with anti-HIV drug treatment, anemia associated with sickle cell disease, anemia associated with beta-thalassemia, anemia associated with cystic fibrosis, anemia associated with pregnancy, anemia associated with menstrual disorders, anemia associated with spinal cord injury, anemia associated with space flight and anemia associated with acute blood loss. In another example, the subject is a mammal, such as a human. In yet another example, the composition or formulation includes an EPO polypeptide or an EPO polypeptide conjugate made by a method of the invention.

The following examples are provided to illustrate the methods of the present invention, but not to limit the claimed invention. Although focused on the exemplary polypeptide EPO, a person of skill in the art will appreciate that the described procedures can also be used to isolate polypeptides other than EPO. Exemplary polypeptides suitable for use with the methods of the invention are described herein, above.

### EXAMPLES

### Example 1

### Determination of Endoglycanase Activity

Samples to be analyzed for endoglycosidase (endoh activity) were diluted 1:1 with glycerol, vortexed and optionally stored at −20° C. to preserve activity prior to analysis. The total sample volume ranged from 8 to 80 mcL, but was typically 40 mcL. The samples were buffer exchanged into 50 mM MES, 50 mM NaCl, pH 6.0 using 10,000 MWCO regenerated cellulose spin filters in a 96 well format. The samples were transferred to a 96-well filter plate and diluted to 300 mcL with 50 mM MES, 50 mM NaCl, pH 6.0 buffer, and centrifuged to near dryness (3000 g, 2×90 min). A second wash was performed by reconstituting with 100 mcL of the same buffer and then centrifuging to near dryness (3000 g, 90 min). The samples were re-diluted with 50 mM MES, 50 mM NaCl, pH 6.0 buffer to a volume of 80 mcL.

EPO substrate, 20 mcL at 1 mg/mL, was then added and the samples were incubated at 30° C. for 18 hours. After incubation, NA2 (asialo, galactosylated, biantennary complex N-glycan) carbohydrate standard (10 mcL, 20 mcg/mL) was added as an internal standard. The samples were then centrifuged (3000 g, 90 min). Glycans released from the EPO substrate and the internal standard glycan were collected in the filtrate. The filtrate was evaporated to dryness on a Speedvac (2 hrs) and derivitized with 2-aminobenzoic acid. Ten microliters of a solution of 2-aminobenzoic acid (50 mg/mL) and sodium cyanoborohydride (60 mg/mL) in 3/7 HOAc/DMSO were added to each sample and the samples were heated at 65° C. for 3 hours. The fluorescently labeled glycans were cooled to room temperature and diluted to 50 mcL volumes with 80% ACN.

Fluorescently labeled endoglycosidase-released glycans and internal standard glycans were separated by normal phase HPLC (20 mcL injection) using an amino column (Shodex Asahipak NH2P-50 4D, 4.6 mm×150 mm). A mobile phase gradient from high to low organic composition was used (buffer B: 2% HOAc, 1% THF in CAN; buffer A: 5% HOAc, 1% THF, 3% TEA in water). The gradient was as follows: wash with 80% B for 10 min at 2.5 mL/min, 80-50% B over 15 min at 2 mL/min, wash at 5% B at 1.5 mL/min, and re-equilibration with 80% B at 2.5 mL/min. The total run time was 35 minutes. Fluorescence of the eluant was monitored using an excitation wavelength of 330 nm and emission detection at 420 nm. The endoglycosidase activity was determined based on the peak area ratio of the enzymatically released glycans to the internal standard (representing 0.122 nmoles of 2-aminobenzoic acid-derivitized glycan). The number of observed nmoles of endoglycosidase-released glycan, the initial sample volume and incubation time were used to calculate activity in units. One unit is defined by the amount of endoglycosidase needed to release 1 micromole of glycan from EPO (20 mcg/100 mcL) per minute at 30° C., pH 6.0. The endoglycosidase assay is illustrated in FIG. 3.

### Example 2

### Determination of Proteolytic Activity

Proteolytic activity in EPO fermentation and process samples was determined using an assay described by Slack er al. (J. Gen. Virol. 1995, 76, 1091-1098) or modified versions thereof. EPO process samples were diluted with water to a final volume of 300 mcL (typically 3 parts sample: 1 part water; but as high as 1 part sample: 9 parts water for samples with a high protease content). A series of aqueous dilutions for an EPO harvest reference control sample were also prepared (100%-3%). Diluted samples and controls (60 mcL) were added to individual wells of 384 deep well microplates in duplicates containing 60 mcL of 200 mM sodium citrate, pH 5.4, 6 M urea, 10 mM EDTA, 10 mM cysteine (mock reactions) or 60 mcL of 200 mM sodium citrate, pH 5.4, 6 M Urea, 10 mM EDTA, 10 mM cysteine with 0.4% azocasein that had been warmed to 32° C. Plates were sealed and inverted 6 times to mix the contents. The plates were centrifuged briefly (1000×g, 10 sec, rt) to return the contents to the base of each well and incubated at least 1 hour at 32° C. with shaking at 350 rpm.

After not more than 18 hours incubation, the reactions were quenched by addition of 50% TCA solution (30 mcL). The plates were sealed and mixed by inversion (6 times). The precipitated protein was pelleted by centrifugation (3220×g, 10 min, 4° C.). Samples of each supernatant (85 mcL) were transferred to 384-well 1.2 micron filter plates and centrifuged into 384-well polystyrene flat-bottomed collection plates (3000×g, 5 min, rt). The absorbance was read at 350 nm. A standard curve was generated by plotting the absorbance readings of the control samples vs. dilution (A350 nm vs. % sample) using a four parameter logistic function of the plate reader software. Absorbance readings were normalized to a reference sample (100%) added to the microplate assay well. The activity of the EPO process sample was determined by comparison to the standard curve with correction for dilution.

### Example 3

### Polypeptide Harvest and Capture from Insect Cell Culture Liquid

In this experiment, insect cell culture liquid at 67 hours post-infection was clarified by pumping the bioreactor contents directly onto two 0.45 micron hollow fiber cartridges. The feed stream was concentrated approximately 10-fold and the retentate was diafiltered with two diavolumes to maximize polypeptide recovery. The hollow fiber permeate stream was loaded in real time onto two chromatography columns connected in series. The first column included a mixed-mode anion exchange filtration medium (Capto Adhere). The second column contained an affinity capture resin (Capto Blue). At the conclusion of the filtration, the columns were washed with low conductivity buffer and the Capto Adhere column was disconnected and removed. The polypeptide was then eluted from the Capto Blue resin with 2 M KCl in a phosphate buffer at pH 7.0. This process when performed at a 15 L fermentation scale, resulted in a 45-55% recovery of EPO (as an exemplary polypeptide) from the insect cell culture liquid. The processing was completed in 2 hours and 18 minutes (hollow fiber feed to collection of the Capto Blue elution pools) and provided EPO in approximately 30% purity while removing 99.6% of the endoglycosidase activity and 97.48% of the protease activity contained in the culture liquid. Results are summarized in Table 4, below.

### 3.1. Methods

Process samples were stored at −20° C. prior to analysis for protein concentration, proteolytic activity and endoglycosidase activity. Total protein concentration was determined measuring absorbance at 280 nm (A280) or by using a Bradford Protein Assay kit according to manufacturer's instructions.

EPO concentration was measured by ELISA using a commercially available monoclonal antibody directed against human EPO, biotinylated anti-human EPO antibody, streptavidin-horseradish peroxidase in combination with 1-Step Turbo TMB-ELISA reagent. Reactions were stopped with 1 N sulfuric acid and the OD was read at 450 nm and 600 nm. A standard calibration curve was generated for each microplate and used to determine the EPO concentration in each sample.

In other instances, EPO concentration was determined using reverse phase HPLC. In one example separation was effected using four coupled Onyx monolithic C8 columns (100 mm×4.6 mm) or equivalent Chromolith Performance RP-8E columns using the following buffer solutions: A: 0.1% TFA in water; B: 0.09% TFA in acetonitrile. Filtered (0.2 micron) EPO samples (100 mcL) were injected onto the series of columns equilibrated at 40% B. After injection, the columns were washed with 40% B for 4 minutes and then eluted with a gradient of 40-50% B over 24 minutes at a flow rate of 1.5 mL/min. Protein was detected at 214 nm. The EPO peak area was integrated and the concentration was calculated based on a calibration curve that had been prepared by analysis of EPO calibration standards.

SDS PAGE analyses were performed under reducing conditions. Silver stained gels were prepared using Wako Silver Stain Kit following manufacturer's instructions. See Blue Plus-2 molecular weight marker was used as a standard on each gel. The protein bands were visualized and scanned with an HP Scanjet 7400C.

### 3.2. Fermentation Harvest Sampling

15.5 L of a freshly harvested baculoviral Sf9 EPO fermentation culture (67 hours post-infection, pH 6, conductivity: 9 mS/cm, cell density: 8.68×10⁶ cells/mL, 95.0% cell viability) was sampled (4×1 mL) for EPO content (ELISA, RP-HPLC), protease activity, total protein content (Bradford) and endoglycosidase activity to establish base-line values. Samples were centrifuged at 1000×g for 5 minutes to remove intact cells and the resulting supernatants were stored at −20° C. prior to analysis. Results are summarized in Table 4, below.

### 3.3. Clarification of Cell Culture Liquid by Hollow Fiber Filtration

Two 0.45 micron hollow fiber cartridges (850 cm² each) cleaned with 0.5 N NaOH, 0.1 N NaOH, 20% ethanol and stored in 0.1 N NaOH were connected in series to a Cole Parmer peristaltic pump with LS pump drive that had been calibrated to 2.4 L/min. The retentate line was led back to a 2.5 L retentate reservoir. The upper permeate outlet on each hollow fiber cartridge was connected to one of two Watson Marlow 505S peristaltic pumps that had both been calibrated to 140 mL/min (19 rpm for both) to operate in flux control at approximately 100 LMH (compare FIG. 2). The utilized hollow fiber process parameters are summarized in Table 2, below:

TABLE 2
Hollow Fiber Parameters for the Clarification of 15.5 L EPO
Culture Liquid

Process Parameters

| | |
|---|---|
| Hollow Fiber Membranes | Two 850 cm² polysulfone membrane |
| | cartridges, 0.45 micron pore size |
| Membrane area, m² | 2 × 850 cm² = 0.17 m² |
| Shear rate | 8000/sec |
| Crossflow | 2.4 L/min |
| Flux | 90-100 LMH (250 mL/min) |
| Retentate Volume (L) | 1.55 L (after 10x concentration) |
| Equilibration/Diafiltration Buffer | 50 mM MES, ~50 mM NaCl, pH 6.0, |
| | 9 mS/cm |
| Diafiltration Criteria | ~2 DF volumes |
| Total Processing Time | Approximately 1 hour |
| Temperature (° C.) | Room temperature (20° C.) |

The entire system and cartridges were flushed with water (8 L) and then 50 mM MES, 50 mM NaCl, pH 6.0 (8.9 mS/cm) prior to processing. The retentate reservoir was filled with fresh fermentation culture and was continually topped off with the remaining harvest material throughout the filtration process. The culture liquid was pumped through the hollow fiber cartridges at 2.4 L/min (8000/sec shear). The retentate pressure between the two hollow fiber cartridges and after the second cartridge was recorded with time and permeate volume. The retentate pressure never exceeded 15 psi. The feed pressure was between 10 and 20 psi. The permeate flow rate was measured at a total of 250 mL/min corresponding to a flux of 90 LMH. The volume of the culture liquid was concentrated to 1.55 L. The hollow fiber retentate was diafiltered two times using 1 L buffer [50 mM MES, 50 mM NaCl (pH 6.0, 8.9 mS/cm)] for each diafiltration step to maximize peptide recovery.

The total processing time for this operation was 64 minutes. Filtrate fractions were loaded directly onto Capto Adhere/Capto Blue columns as described below. Individual samples of permeate from each diavolume as well as the final hollow fiber retentate were analyzed for EPO content (ELISA, RP-HPLC), protease activity, total protein content (Bradford) and endoglycosidase activity. Results are summarized in Table 3, below.

TABLE 3
Hollow Fiber Process Results

| | | | | | Total |
|---|---|---|---|---|---|
| | | | Total | | Protein |
| | Volume | EPO Conc | EPO | Protease | (Bradford) |
| Process Step | (mL) | (mcg/mL) | (mg) | (AU/mL) | (mg) |
| Fermentation | 15500 | 24.71 | 382.96 | 10.78 | 1777.1 |
| Harvest (67 hpi*) | | | | | |
| Hollow Fiber | 16000 | 16.97 | 271.58 | 11.33 | 2530.1 |
| Permeate Pool | | | | | |
| (with diavolumes | | | | | |
| 1-2) | | | | | |
| Diavolume 1 | 1000 | 18.31 | 18.31 | 14.24 | 353.1 |
| Diavolume 2 | 1000 | 13.3 | 13.3 | 17.79 | 398.3 |
| Hollow Fiber | 1500 | 2.12 | 3.19 | >32.94 | 988.8 |
| Retentate | | | | | |

### 3.4. Capto Adhere/Capto Blue Chromatography

A BPG 100 column (10 cm id) was packed to a 10 cm bed height using 880 mL Capto Adhere resin according to manufacturer's instructions. An Omn±50.5 mm column was packed to a bed height of 11 cm with 220 mL of Capto Blue resin according to manufacturer's instructions. The hollow fiber permeate and the diafiltration fractions were pumped onto the equilibrated Capto Adhere (10 cm id×10 cm, 800 mL)/Capto Blue (5.05 cm id×11 cm, 220 mL) column assembly (depicted in FIG. 2) using a LC pump ramping up to a flow rate of 280 mL/min.

The chromatography system was equipped with two detectors to monitor the eluant at 214 and 280 nm. In-line gauges monitored the pressures between the pump and the top of the Capto Adhere column (P1), between the Capto Adhere and Capto Blue columns (P2) and between the Capto Blue column and the UV flow cells (P3). The total system pressure (at the pump) was detected by the LC system. The columns were washed together in series with 7 L of 50 mM MES, 50 mM NaCl, pH 6.0 (8.9 mS/cm). The pump was then stopped and the Capto Adhere column was removed from the system.

### Capto Blue Elution

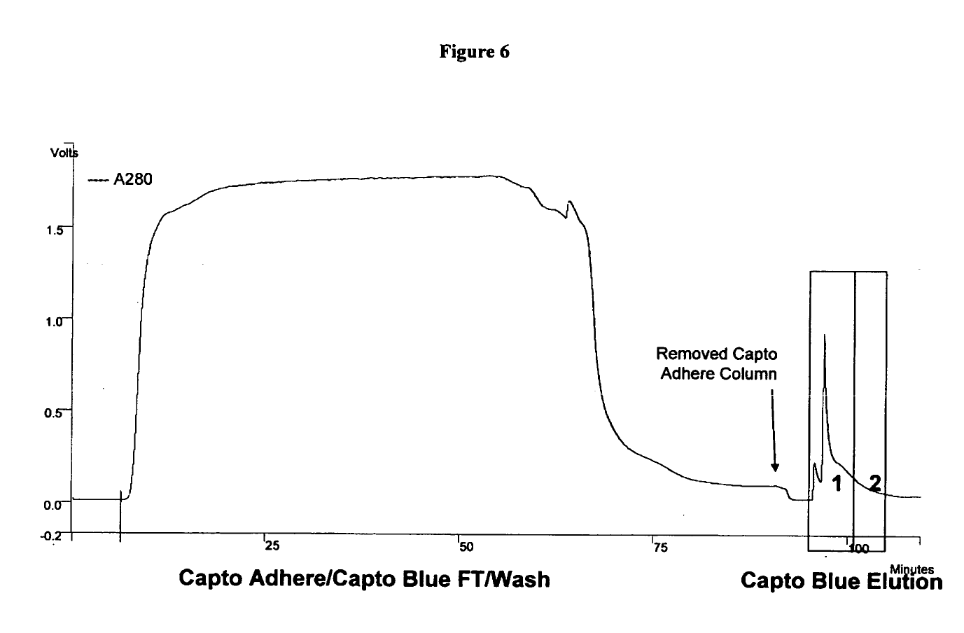

The Capto Blue column was washed with an additional 210 mL (1 CV) of 50 mM MES, 50 mM NaCl, pH 6.0 (8.9 mS/cm) buffer. The Capto Blue column was eluted with 50 mM sodium phosphate, 2 M KCl, pH 7.0 (1.6 L) at a flow rate of 140 mL/min. The EPO product elution was collected as two fraction pools. The elution profile is shown in FIG. 6. The elution pools were sampled for EPO content (ELISA, RP-HPLC), total protein (Bradford) as well as endoglycosidase and protease activities. Results are summarized in Table 4, below. In Table 4, total protein content was determined using the Bradford assay. The flow through and wash fractions were sampled and analyzed by ELISA for EPO breakthrough. No significant breaktrough was detected.

TABLE 4
Summary of Capto Adhere/Capto Blue Process Results

| | | | | EPO Purity | |
|---|---|---|---|---|---|
| | | Protease | EndoH | ELISA | EPO |
| | Volume | Recovery | Recovery | (mg EPO/mg | Recovery |
| Elution Pool | (ml) | (%) | (%) | total protein) | (%) |
| HF Permeate | | | | | 70.9 |
| Main Peak | 1000 | 1.68 | 0.04 | 34% | 53.3 |
| (1) | | | | | |
| Peak Tail (2) | 600 | 0.85 | 0.0 | 43% | 12.0 |
| 1 + 2 | 1600 | 2.52 | 0.04 | 35% | 65.3 |

### Capto Adhere Elution

The Capto Adhere column was reconnected to the LC system and the Capto Blue column was removed. The Capto Adhere column was eluted with 50 mM MES, 1 M NaCl, pH 6 (3.6 L, 4.5 CV) at a flow rate of 200 mL/min. The entire elution peak was collected as one fraction (2 L) which was dark brown in color. The elution peak was sampled and assayed for EPO content by ELISA and analyzed for endoglycosidase and protease activities.

### Example 4

### Optimization of Polypeptide Harvest and Capture

The polypeptide purification steps described in Example 3, above were developed by evaluating various methods for the removal of cell debris and a large panel of capture resins. Experiments were performed to identify robust harvest conditions and chromatographic capture and elution conditions for the rapid concentration of polypeptides (e.g., EPO) from insect cell culture (e.g., infected with baculovirus) scalable to industrial scale (e.g., at least 5000 L fermentation volumes). The selection criteria for suitable process steps included the following aspects directed at overall polypeptide recovery: a) polypeptide stability, b) prevention of protein precipitation and c) reduction of endoglycosidase and protease activities. Optimization experiments were conducted at an experimental 15 L fermentation scale.

### 4.1. Optimization of Cell Culture Clarification

Fresh EPO fermentations in Sf9 cells were produced in 1 L shaker flasks or a 15 L bioreactor for the development of cell clarification methods. At the time of cell culture harvest (67 hours after Baculovirus infection of the cell culture) the cell viabilliy was typically 90% or greater, but the cells were swollen and exceedingly fragile. Three possible methods for removal of insect cell debris were compared: depth filtration using Cuno 30SP filters, batch centrifugation, and hollow fiber filtration using GE PES membranes. All three methods resulted in Sf9 cell lysis and produced identical feed streams as shown by RP-HPLC analysis. Since depth filtration was associated with fouling of the dead-end filters, it was not the best choice for the processing of large volumes. Hollow fiber filtration was selected for further optimization because this method allowed for rapid large-scale processing, combination with capture chromatography steps in a continuous process module, and direct scale-up of experimental conditions.

Three membrane pore sizes were tested (0.2 micron, 0.45 micron and 0.65 micron) and shear rates of 2000/sec to 16,000/sec were compared (flux varied from 20 to 200 LMH). Cell viability was measured using a Guava assay. Results are summarized in Table 5, below:

TABLE 5
Summary of the Performances of Various Hollow Fiber Membranes

| Pore | | | | | Conc. | Cell |
|---|---|---|---|---|---|---|
| Size | Shear | Flux | Time | Membrane | Factor | Viability |
| (μm) | (sec⁻¹) | (LMH) | (min) | Area (cm²) | (x) | (%) |
| 0.65 | 2,000 | 30 | 70 | 50 | 2.45^{b)} | 2 |
| 0.65 | 8,000 | 200 | 30 | 50 | 20 | 8 |
| 0.65 | 8,000 | ~60^{a)} | 210 | 50 | 2 | 2 |
| 0.65 | 10,000 | 200 | 80 | 50 | 10 | 7 |
| 0.65 | 8,000 | 120 | 35 | 110 | 20 | 15 |
| 0.45 | 4,000 | 20 | 140 | 50 | 2 | 8 |
| 0.2 | 10,000 | 200 | 250 | 50 | 10 | 8 |

^{a)}Experiment was run at constant TMP of 0.2 bar
^{b)}TMP reached 1.5 bar at only 2.45 x concentration of the retentate and the run was aborted.

Both 0.45 micron and 0.65 micron pore-sized cartriges performed well without fouling. At shear rates of at least 8000/sec the processing times for 10-20× concentration of the retentate became less than 1 hour. Cell viability was found to drop as processing time and retentate concentration increased. Upon ten-fold concentration of the feed volume at least 80% of the cells had lysed and the cell viability dropped to zero when the retentate was diafiltered with fermentation media. More EPO was released as the cells were lysed with processing. Approximately 20-30% of the product polypeptide was found to be intracellular. More protease was also released. Since a low shear setting (2000/sec) required longer processing times, the membrane area that would be required to compensate for this factor would be prohibitive at 5000 L scale.

The shear/flux settings were optimized using membranes with 0.45 and 0.65 micron pore sizes. It was discovered that shear rates of 8000/sec led to high recovery of EPO and no fouling of the membranes. Processing at shear rates of 10,000/sec or 16,000/sec provided equally high EPO recoveries, but did not provide significant time saving advantages and require greater pump capacity. The 0.45 micron filters performed more consistently with good average flux (100-300 LMH) and low TMP (0.1-0.4 bar). Subsequent experiments were carried out utilizing a permeate pump to target operation at a controlled flux of 100 LMH. It was determined that pump capacity could be further conserved by utilizing membranes with a longer path length. Hollow fiber cartridges with 60 cm path lengths (both 0.45 and 0.65 micron pore size) performed well in flux control at 100 LMH with average TMP's of approximately 0.3 bar.

The membrane area required to complete the clarification processing within the target one hour time-frame was typically 80 L/m². However, a capacity experiment showed that the culture liquid feed volume could be doubled to 160 L/m², with no adverse processing effects. No membrane fouling or pressure increases were observed. This suggested that the membrane capacity is not exhausted when operating at 80-100 L fermentation volume/m² membrane.

Settling of the intact cells in the EPO fermentation harvest material (>90% viability) by gravity prior to hollow fiber processesing was briefly examined. However no performance improvement was observed in processing the settled supernatant and the aging of the cells during the time required for settling (2.5 hrs), led to a drop in cell viability and additional lysis. It was concluded that hollow fiber processing should commence immediately following the harvest of the cells from the bioreactor. In addition, lysis of additional cells during processing increased overall EPO recoveries.

In nearly all hollow fiber processing experiments the EPO fermentation harvest volume was concentrated 10-fold and the final retentate was diafiltered (1-5 times) to maximize EPO recovery. Experiments showed that greater concentration of the retentate (15-20-fold) resulted in rapid elevation of the feed pressure (from well below 10 psi to nearly 20 psi) therefore the practice was discontinued. Typically, about 7% (RP-HPLC) of the EPO remained in the retentate after 10× concentration. After one equal volume diafiltration wash it was reduced to approximately 3%. At the 15 L scale, less than 1% of the harvested EPO was lost in the hollow fiber retentate after 10× concentration and two-fold diafiltration (Table 3). Additional diafiltration of the retentate only added to the volume of hollow fiber permeate to be processed while recovering very little additional EPO.

### 4.2. Optimization of Polypeptide Capture

The EPO hollow fiber permeate feed stream, although clarified significantly, still contained fermentation media components (including yeastolate, and lipid mix: cholesterol, cod liver oil, and Pluronic F68), DNA and host cell protein along with EPO. Hence, a suitable capture resin had to be capable of accomodating a slightly viscous feed stream with high flow rates at a 1000 L-5000 L scale. Capture resins with large particle sizes and high binding capacities were considered for this step including hydrophobic interaction (HIC), ion-exchange (anion and cation exchange), mixed mode, affinity resins and chelation resins. The resins were screened for their ability to efficiently capture and efficiently elute EPO with high polypeptide recovery. Conditions that captured the degradative enzymes (proteases and endoglycosidases) while allowing the EPO to flow through in high recovery were also considered. Therefore feed streams, flow through fractions and elution fractions were tested for EPO content (by RP-HPLC and/or ELISA) and protease activity. Promising conditions were repeated and tested for endoglycosidase removal. Experiments were run in parallel using EPO hollow fiber permeate (from the same process batch if at all possible) that had been previously frozen. It was discovered that freeze/thaw dramatically reduced endoglycanase activity leading to variable results for endoglycanase removal at this stage.

HIC resins Capto Phenyl Sepharose (high and low ligand substitution) and Capto Butyl Sepharose were evaluated. Sodium chloride, sodium citrate and sodium sulfate were tested as binding salts (0-4 M) at pH 7.5 and 5.7. Under the tested conditions, EPO could not be bound effectively by these resins. In addition, proteases and endoglycosidases were not significantly removed from the EPO-containing fractions.

The above results compounded with the challenge of adding salt and increasing load conductivities. Hence, alternative capture procedures were evaluated. Ion-exchange resins Q and SP Sepharose Big Beads and Capto S were tested. Clarified EPO harvest samples were loaded at pH's ranging from 4.5 to 7.5, with and without dilution with water (1:1) to optionally lower the conductivity of the load sample from approximately 9 mS/cm to approximately 5.5 mS/cm. In all cases EPO was not bound sufficiently by the S, SP or Q resins. In addition, protease activity appeared to track with the EPO. An additional set of experiments incidated that EPO from hollow fiber permeate would not bind to either Capto S or SP Sepharose Big Bead resins at loading conductivities as low as 2 mS/cm. Hence, these cation exchange resins were not investigated further for initial capture of EPO.

The mixed mode resin Capto MMC, which has weak cationic exchange capabilities coupled with hydrophobic and hydrogen bonding functionalities is reported by the manufacturer to be tolerant to high conductivity feed streams to capture polypeptides and was tested as an alternative. The EPO from frozen hollow fiber permeate could be effectively captured on the Capto MMC resin between pH 4.5 to 7.5. However, conditions for efficient elution of EPO could not be found. Increasing and decreasing salt (NaCl) concentrations with steps and linear gradients, low and high pH elution (3 and 10), excipients including alcohols (20% ethanol, 10% isopropanol), 10% ethylene glycol, 50 mM glycine and 0.5 M arginine could only elute EPO with a recovery of about 50%.

Experiments with Blue Sepharose (Fast Flow) resin showed that EPO from hollow fiber permeate could be effectively captured without any adjustment to the pH or the conductivity (pH 6, ˜9 mS/cm) of the feed steam. EPO eluted in at least 70% recovery with 1-2 M NaCl.

Capto Blue resins from GE (low ligand substitution ˜9% and high ligand substitution ˜15%) were tested because of their flow characteristics, which are more appropriate for the crude EPO feed stream, as well as their more stable ligand linkage making them more amenable to requisite manufacturing sanitization methods. Blue Sepharose Fast Flow HighTrap columns (1 mL) and Capto Blue resin from GE Healthcare (packed in 0.5 cm×5 cm, 1 mL columns) were pre-equilibrated to 50 mM MES buffer with NaCl, pH 6, 9 mS/cm. Samples of EPO cell culture clarified by hollow fiber filtration (0.45 micron) (25 mL, pH ˜6, ˜9 mS/cm) were loaded onto the columns. Columns were eluted (1 mL/min) with multiple step gradient elutions as indicated. The Capto Blue resins both efficiently bound the EPO without adjustment of pH or conductivity, however only the low ligand density resin allowed efficient recovery of the EPO as shown in Table 6, below.

TABLE 6
Capture of EPO from Hollow Fiber Permeate by Blue Sepharose
Fast Flow, Capto Blue (Low Ligand Substitution) and Capto
Blue (High Ligand Substitution) and Step-Elution Using NaCl

| | | EPO Recovery |
|---|---|---|
| Resin | Loading Conditions | (HPLC) |
| Blue Sepharose FF | pH 5.82 | FT/Wash: 0% |
| | Cond. 9.31 ms | 0.5M: 0% |
| | | 1M: 17.5% |
| | | 2M: 54.7% |
| | | 3M: 0% |
| | | 4M: 0% |
| Blue Sepharose FF | pH 5.82 | FT/Wash: 0% |
| | Cond. 9.31 ms | 0.5M: 0% |
| | | 1M: 73.1% |
| | | 2M: 0% |
| Capto Blue I | pH 5.82 | FT/Wash: 0% |
| Low sub | Cond. 9.31 ms | 0.5M: 0% |
| | | 1M: 71.3% |
| | | 2M: 12.3% |
| Capto Blue II | pH 5.82 | FT/Wash: 0% |
| High sub | Cond. 9.31 ms | 0.5M: 0% |
| | | 1M: 0% |
| | | 2M: 0% |
| | | 3M: 5.2% |

A panel of elution salts and excipients (NaCl, KCl, arginine, sodium sulfate, sodium citrate, glycine, ethylene glycol, ethanol) and various pH conditions (6-9.5) were screened to maximize EPO recovery. The best results were observed with 2 M KCl or 2 M arginine at all of the pH's tested. Results are summarized in Tables 7 and 8, below.

In Tables 6 to 8, Q Sepharose Big Beads (Q-BB) or Capto Blue resin from GE Healthcare (low ligand substitution) was packed in 0.5 cm×5 cm, 1 mL columns and pre-equilibrated with 50 mM MES buffer with NaCl, pH 6, 9 mS/cm. Samples of EPO culture liquid clarified by hollow fiber filtration (0.45 micron) (25 mL, pH ˜6, ˜9 mS/cm) were loaded onto the columns. Columns were washed as indicated and eluted (1 mL/min) using single step (S) or multiple step (MS) elution as indicated. Abbreviations: FT=Flow through fraction, HF=hollow fiber. EPO recovery was determined by RP-HPLC. Protease recovery/removal was determined by protease assay.

TABLE 7
Elution of EPO (Hollow Fiber Permeate) from Capto Blue (Low Ligand
Substitution)

| | | | Recovery | Residual |
|---|---|---|---|---|
| Resin | Load Conditions | Elution Conditions | HPLC (%) | Proteolytic Activity |
| Capto | pH 5.82 | 2M NaCl, | FT/Wash: 3.5% | FT/Wash: <12.1% |
| Blue LS | Cond: 9.3 ms | pH 6 | Blue Elute: 75.5% | Blue Elute: 4.1% |
| Capto | pH 5.82 | 2M KCl, | FT/Wash: 3.5% | FT/Wash: 12.1% |
| Blue LS | Cond: 9.3 ms | pH 6 | Blue Elute: 85.3% | Blue Elute: 6.9% |
| Capto | pH 5.82 | 2M Arginine, | FT/Wash: 3.1% | FT/Wash: <12.1% |
| Blue LS | Cond: 9.3 ms | pH 6 | Blue Elute: 88.6% | Blue Elute: 36% |
| Capto | pH 5.82 | 2M Glycine, pH 6 | FT/Wash: 3.4% | FT/Wash: <12.1% |
| Blue LS | Cond: 9.3 ms | | Blue Elute: BLD | Blue Elute: 0% |
| Capto | pH 5.82 | 2M NaCl, 0.5 M | FT/Wash: 3.7% | FT/Wash: <12.1% |
| Blue LS | Cond: 9.3 ms | Arginine, pH 6 | Blue Elute: 81.8% | Blue Elute: 40.1%** |
| Capto | pH 5.82 | 2M NaCl, 20% Ethanol, | FT/Wash: 3.7% | FT/Wash: <12.1% |
| Blue LS | Cond: 9.3 ms | pH 6 | Blue Elute: 34% | Blue Elute: 76.6% |
| Capto | pH 5.82 | 2M NaCl, 20% | FT/Wash: 3.3% | FT/Wash: 12.1% |
| Blue LS | Cond: 9.3 ms | Ethylene Glycol, pH 6 | Blue Elute: 79.5% | Blue Elute: 25.7% |
| Capto | pH 5.82 | 2M NaCl, pH 6 | FT/Wash: 0% | FT/Wash: <12% |
| Blue LS | Cond: 9.3 ms | | Blue Elute: 75.4% | Blue Elute: 3.8% |
| Capto | pH 5.82 | 2M KCl, | FT/Wash: 4.2% | FT/Wash: 14.3% |
| Blue LS | Cond: 9.3 ms | pH 6 | Blue Elute: 83.7% | Blue Elute: 6.4% |
| Capto | pH 5.82 | 2M Arginine, | FT/Wash: 4.4% | FT/Wash: 16.6% |
| Blue LS | Cond: 9.3 ms | pH 6 | Blue Elute: 87.3% | Blue Elute: >88.5% |
| Capto | pH 5.82 | 1.6M Na Citrate, | FT/Wash: 3% | FT/Wash: 12.4% |
| Blue LS | Cond: 9.3 ms | pH 6 | Blue Elute: BLD | Blue Elute: <3.4% |
| Capto | pH 5.82 | 1M KCl, 1 M Arginine, | FT/Wash: 5.2% | FT/Wash: <12% |
| Blue LS | Cond: 9.3 ms | pH 6 | Blue Elute: 87.9% | Blue Elute: >83.5% |
| Capto | pH 5.82 | 0.2M KCl, 1.8M | FT/Wash: 6.1% | FT/Wash: 13.2% |
| Blue LS | Cond: 9.3 ms | Arginine, pH 6 | Blue Elute: 81.6% | Blue Elute: >83.5% |
| Capto | pH 5.82 | 1.8M KCl, 0.2M | FT/Wash: 4.4% | FT/Wash: 14.5% |
| Blue LS | Cond: 9.3 ms | Arginine, pH 6 | Blue Elute: 82.2% | Blue Elute: 16.8% |

TABLE 8
Elution of EPO (Hollow Fiber Permeate) from Capto Blue (Low Ligand
Substitution)

| | | Elution Conditions | EPO |
|---|---|---|---|
| Resin | Loading Conditions | (Step-elution) | Recovery (%) |
| Q-BB | pH 5.82 | EPO in FT | FT/Wash: 104.5% |
| | Cond: 9.3 ms | Elute: 1M NaCl, pH 6 | Elution: BQL |
| Capto Blue LS | pH 5.82 | 2M KCL, pH 6 | FT/Wash: BLD |
| | Cond: 9.3 ms | | Blue Elution: 78% |
| Capto Blue LS | pH 5.82 | 2M Arginine, pH 6 | FT/Wash: 6.5% |
| | Cond: 9.3 ms | | 0.2M: 5.6% |
| | | | 0.4M: 75.8% |
| | | | 0.6M: 0.8% |
| | | | 0.8M: BLD |
| | | | 1M: BLD |
| | | | 2M: BLD |
| Capto Blue LS | pH 5.82 | Wash: 72 mM NaCl, | FT/Wash: 6% |
| | Cond: 9.3 ms | 20% Ethanol then | Ethanol Wash: BLD |
| | | Elute: 2M Arg, pH 6 | Elution: 81.1% |
| Capto Blue LS | pH 5.82 | Wash: 0.25M NaCl, | FT/Wash: 5.6% |
| | Cond: 9.3 ms | 20% Ethanol then | Ethanol. Wash: BLD |
| | | Elute: 2M Arg, pH 6 | Elution: 77.5% |
| Capto Blue LS | pH 5.82 | Wash: 0.5M NaCl, | FT/Wash: 5% |
| | Cond: 9.3 ms | 20% Ethanol then | Eth. Wash: 5.4% |
| | | Elute: 2M Arg, pH 6 | Elution: 64.1% |

Protease activity was reduced but not completely eliminated from the EPO elution pool by Capto Blue. In addition, endoglycosidase activity was not separated from the EPO pool by the Capto Blue resin. Hence, anion exchange conditions, which might capture the degradative enzymes and allow EPO to flow through, were screened for their potential to be used with the Capto Blue capture conditions. Q Sepharose Big Beads resin, Capto Q, Capto Adhere (mixed mode anion-exchange) and Sartobind Q resins were tested. The feed pH ranged between pH 5 and pH 8.5 and the conductivity ranged from 5 mS/cm to 9 mS/cm. EPO recovery was high (>90%) under all conditions tested. The best protease reduction was observed using Q Sepharose Big Beads and Capto Adhere resins at pH 5.7 (no pH adjustment) at reduced conductivity (5 mS/cm) when small volumes of EPO hollow fiber permeate were loaded (5-25 CV). Both resins reduced endoglycosidase activity.

Additional examples describing methods and procedures useful in the methods of the invention are described in commonly owned U.S. patent application Ser. No. 11/396,215 filed Mar. 30, 2006, the disclosure of which is incorporated herein its entirety for all purposes.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of making a composition comprising a recombinant erythroooietin (EPO) polypeptide, wherein said EPO polypeptide is expressed in an insect cell, said composition essentially free of endoglycanase activity, said method comprising:
(a) subjecting a mixture comprising said EPO polypeptide to mixed-mode chromatography comprising:
(i) contacting said mixture and a mixed-mode chromatography medium comprising a mixed-mode ligand having a quatemary amino group; and
(ii) eluting said polypeptide from said mixed-mode chromatography medium, thereby generating a flow-through fraction comprising said polypeptide, thereby forming said composition.

2. The method of claim 1, wherein said mixed-mode ligand further comprises a hydrophobic moiety selected from linear or branched unsubstituted alkyl, unsubstituted aryl, alkyl-substituted aryl, unsubstituted heteroaryl and alkyl-substituted heteroaryl.

3. The method of claim 1, wherein said mixed-mode ligand further comprises a moiety including at least one hydroxyl group.

4. The method of claim 1, wherein said mixed-mode chromatography medium is Capto Adhere.

5. The method of claim 1, further comprising:
(b) subjecting said flow-through fraction comprising said EPO polypeptide to dye-ligand affinity chromatography comprising:
(i) contacting said flow-through fraction with a dye-ligand affinity chromatography medium under conditions sufficient for said EPO polypeptide to reversibly bind to said dye-ligand affinity chromatography medium; and
(ii) eluting said EPO polypeptide from said dye-ligand affinity chromatography medium, thereby generating an eluate fraction comprising said EPO polypeptide, said eluate fraction essentially free of endoglycanase activity.

6. The method of claim 5, wherein said dye-ligand affinity chromatography medium comprises Cibacron Blue or an analog thereof.

7. The method of claim 6, wherein said Cibacron Blue is immobilized on a sepharose- or an agarose-based matrix.

8. The method of claim 6, wherein said dye-ligand affinity chromatography medium is Capto Blue.

9. The method of claim 5, wherein said flow-through fraction comprising said EPO polypeptide is contacted with said dye-ligand affinity chromatography medium essentially immediately after elution from said mixed-mode chromatography medium.

10. The method of claim 9, wherein said mixed-mode chromatography and said dye-ligand affinity chromatography are linked in a continuous-flow process module.

11. The method of claim 5, wherein said endoglycanase activity of said eluate fraction is less than about 1% compared to endoglycanase activity of said mixture prior to said mixed-mode chromatography and said dye-ligand affinity chromatography.

12. The method of claim 5, wherein said eluate fraction has a proteolytic activity that is less than about 5% compared to proteolytic activity of said mixture prior to said mixed-mode chromatography and said dye-ligand affinity chromatography.

13. The method of claim 5, wherein said EPO polypeptide in said eluate fraction has a purity of at least about 25% (w/w).

14. The method of claim 5, wherein at least 65% of said EPO polypeptide contained in said mixture is recovered in said eluate fraction after said mixed-mode chromatography and said dye-ligand affinity chromatography.

15. The method of claim 5, further comprising prior to step (a): removing cellular debris from a cell culture liquid comprising said EPO polypeptide, thereby generating said mixture comprising said EPO polypeptide.

16. The method of claim 15, wherein said removing is accomplished using hollow fiber filtration.

17. The method of claim 15, wherein said removing cellular debris, said mixed-mode chromatography and said dye-ligand affinity chromatography are performed in a single-unit operation.

18. The method of claim 5, further comprising: eluting said EPO polypeptide from at least one chromatography medium, which is a member selected from a hydrophobic interaction chromatography medium, a cation exchange chromatography medium and a hydroxyapatite or fluoroapatite chromatography medium.

19. The method of claim 1, wherein said polypeptide comprises a substantially uniform, insect-specific glycosylation pattern.

20. (canceled)

21. The method of claim 1, further comprising: infecting insect cells in an insect cell culture with a recombinant baculovirus comprising a nucleotide sequence encoding said EPO polypeptide, wherein said insect cell culture is supplemented with a lipid mixture prior to said infecting.

22. The method of claim 21, wherein said insect cell culture is supplemented with said lipid mixture at a percentage of total culture volume equivalent to between about 0.5% and about 3% v/v and wherein said insect cell culture is supplemented with said lipid mixture from between about 0.5 hours to about 2.0 hours prior to said infecting.

23. The method of claim 21, wherein said infecting employs a multiplicity of infection between about 10⁻⁸ and about 1.0.

24. The method of claim 21, wherein said lipid mixture comprises: an alcohol, a surfactant, a sterol, a detergent, an anti-oxidant, and a lipid source.

25. The method of claim 21, further comprising: expressing said EPO polypeptide in said insect cells.

26. The method of claim 21, wherein said insect cells are Spodoptera frugiperda cells.

27. A composition made by the method of claim 1.

28. A pharmaceutical formulation comprising the composition of claim 27 and a pharmaceutically acceptable carrier.

29. A method of making a composition comprising a recombinant EPO polypeptide, wherein said EPO polypeptide is expressed in an insect cell, said composition essentially free of endoglycanase activity, said method comprising:
(a) subjecting a mixture comprising said EPO polypeptide to mixed-mode chromatography comprising:
(i) contacting said mixture with a mixed-mode chromatography medium comprising a mixed-mode ligand having a quaternary amino group and at least one moiety selected from a hydrophobic moiety and a moiety comprising a hydroxyl group; and
(ii) eluting said EPO polypeptide from said mixed-mode chromatography medium thereby generating a flow-through fraction comprising said EPO polypeptide,
thereby forming said composition.

30. The method of claim 29, wherein said EPO polypeptide comprises an amino acid sequence according to SEQ ID NO: 1, said sequence optionally having at least one mutation selected from the group consisting of Arg¹³⁹ to Ala¹³⁹, Arg¹⁴³ to Ala¹⁴³ and Lys154 to Ala154.

31. A composition made by the method of claim 29.

32. A pharmaceutical formulation comprising the composition of claim 31 and a pharmaceutically acceptable carrier.

33. A method of making a composition comprising a recombinant erythropoietin (EPO) polypeptide, wherein said EPO polypeptide is expressed in an insect cell, said composition essentially free of endoglycanase activity and essentially free of proteolytic activity, said method comprising:
(a) eluting a mixture comprising said EPO polypeptide from a mixed-mode chromatography medium comprising a mixed-mode ligand having a quaternary amino group and at least one moiety selected from a hydrophobic moiety and a moiety comprising a hydroxyl group, thereby generating a flow-through fraction comprising said EPO polypeptide;
(b) contacting said flow-through fraction with a dye-ligand affinity chromatography medium; and
(c) eluting said EPO polypeptide from said dye-ligand affinity chromatography medium thereby producing an eluate fraction comprising said EPO polypeptide, thereby forming said composition.

34. The method of claim 33, further comprising: irradiating said eluate fraction with UV light in a manner sufficient to effect viral inactivation.

35. The method of claim 33, further comprising passing said EPO polypetide through a membrane, wherein said membrane has a molecular weight cutoff (MWCO) sufficient to remove viral particles.

36. The method of claim 33, further comprising eluting said EPO polypeptide from at least one chromatography medium, which is a member selected from a hydrophobic interaction chromatography medium, a cation exchange chromatography medium and a hydroxyapatite or fluoroapatite chromatography medium.

37. The method of claim 33, wherein said EPO polypeptide comprises a substantially uniform, insect-specific glycosylation pattern.

38. The method of claim 33, wherein said flow-through fraction comprising said EPO polypeptide is contacted with said dye-ligand affinity chromatography medium essentially immediately after elution from said mixed-mode chromatography medium.

39. The method of claim 38, wherein said mixed-mode chromatography and said dye-ligand affinity chromatography are linked in a continuous-flow process module.

40. The method of claim 33, further comprising prior to step (a): removing cellular debris from a cell culture liquid comprising said EPO polypeptide, thereby generating said mixture comprising said EPO polypeptide.

41. The method of claim 40, wherein said removing is accomplished using hollow fiber filtration.

42. The method of claim 40, wherein said removing cellular debris, said mixed-mode chromatography and said dye-ligand affinity chromatography are performed in a single-unit operation.

43. The method of claim 33, further comprising expressing said recombinant EPO polypeptide in an insect cell line.

44. The method of claim 43, wherein said insect cell line is a Spodoptera frugiperda cell line.

45. (canceled)

46. (canceled)

47. The method of claim 33, wherein said EPO comprises an amino acid sequence according to SEQ ID NO: 1 optionally having at least one mutation selected from the group consisting of Arg¹³⁹ to Ala¹³⁹, Arg¹⁴³ to Ala¹⁴³ and Lys¹⁵⁴ to Ala154.

48. The method of claim 33, further comprising: infecting insect cells in an insect cell culture with a recombinant baculovirus comprising a nucleotide sequence encoding said EPO polypeptide, wherein said insect cell culture is supplemented with a lipid mixture prior to said infecting.

49. The method of claim 48, wherein said lipid mixture is supplemented into said insect cell culture at a percentage of total culture volume equivalent to between about 0.5% and about 3% v/v.

50. The method of claim 48, wherein said lipid mixture is added to supplement said insect cell culture from between about 0.5 hours to about 2.0 hours prior to said infecting.

51. The method of claim 48, wherein said infecting employs a multiplicity of infection between about 10⁻⁸ to about 1.0.

52. The method of claim 48, wherein said lipid mixture comprises: an alcohol, a surfactant, a sterol, a detergent, an anti-oxidant, and a lipid source.

53. The method of claim 33, wherein said endoglycanase activity of said eluate fraction is less than about 1% compared to endoglycanase activity of said mixture prior to said mixed-mode chromatography and said dye-ligand affinity chromatography.

54. The method of claim 33, wherein said eluate fraction has a proteolytic activity that is less than about 5% compared to proteolytic activity of said mixture prior to said mixed-mode chromatography and said dye-ligand affinity chromatography.

55. The method of claim 33, wherein said EPO polypeptide in said eluate fraction has a purity of at least about 25% (w/w).

56. The method of claim 33, wherein at least 65% of said polypeptide contained in said mixture is recovered in said eluate fraction after said mixed-mode chromatography and said dye-ligand affinity chromatography.

57. A composition made by the method of claim 33.

58. A pharmaceutical formulation comprising a composition of claim 57 and a pharmaceutically acceptable carrier.

59. A method of enhancing red blood cell production in a mammal, said method comprising administering to said mammal a composition according to claim 31.

60. A method of treating a tissue injury in a subject in need thereof, said injury resulting from ischemia, trauma, inflammation or contact with toxic substances, said method comprising the step of administering to the subject an amount of a composition according to claim 31, effective to ameliorate the damage associated with the tissue injury in said subject.

61. A method of treating anemia, comprising administering a composition according to claim 31 to a subject in need thereof.

62. The method according to claim 61, wherein said anemia is age related anemia, early anemia of prematurity or anemia associated with a member selected from chronic renal failure, cancer chemotherapy treatment, anti-HIV drug treatment, sickle cell disease, beta-thalassemia, cystic fibrosis, pregnancy, menstrual disorders, spinal cord injury, space flight and acute blood lossof treating anemia.
